(19) United States
(12) Patent Application Publication
(10) Pub. No.: US 2023/0272473 A1
(43) Pub. Date: Aug. 31, 2023

(54) METHOD, KIT AND SYSTEM FOR SYNCHRONOUS PRENATAL DETECTION OF CHROMOSOMAL ANEUPLOIDY AND MONOGENIC DISEASE

(71) Applicants: Beijing BioBiggen Technology Co., Ltd., Beijing (CN); BioBiggen IntelManu (Beijing) Tech Co., Ltd., Beijing (CN)

(72) Inventors: Jinglan Zhang, Sugar Land, TX (US); Jianli Li, Bellaire, TX (US); Zhiwei Zhang, Beijing (CN)

(21) Appl. No.: 17/938,570

(22) Filed: Oct. 6, 2022

Related U.S. Application Data
(63) Continuation of application No. PCT/CN2021/112314, filed on Aug. 12, 2021.

(30) Foreign Application Priority Data
Aug. 13, 2020 (CN) 202010815673.8

(51) Int. Cl. C12Q 1/6883; G16B 20/20; G16B 25/20; G16B 30/00

(52) U.S. Cl. CPC C12Q 1/6883; G16B 20/20; G16B 25/20; G16B 30/00; C12Q 2600/156

(57) ABSTRACT

The present disclosure provides a detection method, kit and system for non-invasive prenatal screening of fetal chromosome copy number variation, fetal chromosome microdeletion/microduplication, and/or dominant monogenic variation. The present disclosure further provides a method of designing a targeted capture probe. The detection method is used for non-invasive prenatal screening of fetuses, and compared with the existing detection method for non-invasive prenatal screening, its application scope in clinical genetic detection can be expanded, and the detection accuracy can be improved.

### CROSS-REFERENCE

This application is a continuation application of International Application No. PCT/CN2021/112314, filed Aug. 12, 2021, which claims the benefit of Chinese Patent Application No. 202010815673.8, filed Aug. 13, 2020, which is incorporated herein by reference in its entirety.

### SEQUENCE LISTING

The instant application contains a Sequence Listing which has been submitted electronically in XML format is hereby incorporated by reference in its entirety. Said XML copy, created on Oct. 18, 2022, is named 60999-701_301_SL.xml and is 13,243 bytes in size.

### BACKGROUND

Birth defects may refer to abnormal growth and development of a fetus in the mother's womb, resulting in congenital defects that are already present at birth. With a large population, the number of birth defects in China increases by about 900,000 per year, and the incidence of birth defects is about 5.6% [1]. Birth defects are a major cause of death and disability of infants and young children, and have become a major public health problem affecting the health of the population, and the resultant social and economic burden is heavy.

Genetic factors may be an important cause of birth defects, and both chromosomal abnormalities and monogenic diseases can cause many types of birth defects. Chromosomal abnormalities may comprise copy number abnormalities and structural abnormalities, and the most common copy number abnormality is chromosomal aneuploidy, the incidence of which is about 1/160 at birth [2]. Chromosomal aneuploidy may refer to the difference between chromosome number and the diploid genome (46, XX or 46, XY), such as a gain or deletion of a chromosome. Common genetic diseases of chromosomal aneuploidy include chromosome 21 (T21, Down syndrome), chromosome 18 (T18, Edwards' syndrome), and chromosome 13 (T13, Patau syndrome), which often result in fetal structural abnormalities, multi-organ malformations, and developmental disorders, with high mortality and disability, for which there is no effective treatment. Chromosomal structural abnormalities may include microdeletion and microduplication, and some common ones are 22q11.2, 1p36, 5q deletion syndromes, and the like [2].

In view of the above-mentioned clinical issues associated with chromosomal aneuploidy, screening during pregnancy and prenatal diagnosis may be effective approaches to prevent and control the incidence of birth defects. Traditional screening methods may include serological examinations and imaging examinations, which assess the risk of fetal genetic defects by detecting changes in the levels of various biomarkers in maternal serum at different stages of pregnancy, combined with ultrasound imaging observations, and prenatal diagnosis by placental chorionic villus sampling (CVS) or amniocentesis [3]. The disadvantages of these methods may include the low sensitivity of serological examination (about 69-96%) and the high rate of false positives (about 5%) for the 3 trisomy syndromes mentioned above [4]. In addition, although prenatal diagnosis has high sensitivity and specificity, it may be an invasive detection and may pose certain risk of fetal abortion (about 0.5-1%) [2]. Therefore, there exists a need for improved non-invasive screening techniques to further improve the sensitivity and specificity of analytical methods without increasing the risk of pregnancy, especially to reduce the false-positive and false-negative detection results caused by technical limitations of existing techniques during large-scale clinical application. Such scientific and clinical research directions may improve the clinical effectiveness of prenatal screening for chromosomal abnormality diseases.

The discovery of fetal cell-free DNA (cfDNA) in maternal plasma during pregnancy has driven the development of non-invasive prenatal screening (NIPS) technology and its clinical application [5]. Since 2011, NIPS has been offered nationwide in China to pregnant women as a prenatal screening test, and its sensitivity and specificity, as well as clinical verifiability, have been validated in hundreds of thousands of clinical samples [6]. It may be shown that fetal cell-free DNA is derived from apoptotic cells in fetal placental tissue, that its concentration in maternal peripheral blood varies over time, and that it is rapidly cleared by the mother after delivery [7, 8]. Since fetal cell-free DNA contains fetal genetic information, appropriate detection methods (quantitative PCR, digital PCR, high-throughput sequencing, etc.) can be used to screen for chromosomal abnormalities and assess the risk of fetal genetic defects, and its non-invasive nature can also avoid the risk of maternal miscarriage. The non-invasive prenatal screening (NIPS) that has been widely used for chromosomal aneuploidy can be performed in early pregnancy (9-12 weeks) using maternal peripheral blood as the sample, with simple and safe sampling method, and has high sensitivity (about 97-99%) and low false-positive rate (<0.1%) for chromosomal aneuploidy detection such as T21, T18, and T13, which has been widely validated and recognized by clinical practice [9-13]. The current mainstream NIPS detection method may be based on next-generation sequencing (NGS), which uses the massively parallel sequencing to analyze the depth of the reads of maternal and fetal DNA fragments in a sample, and determines the number of fetal chromosomes of interest with WGS by measuring the ratio of reads in the chromosome of interest to reads on a corresponding diploid reference chromosome. Although this method can effectively detect common chromosomal aneuploidy such as T21, T18 and T13, as well as microdeletions/microduplications of some large segments, the WGS method may be inaccurate in quantifying the proportion of fetal cell-free DNA in practice (especially for female fetuses), which may cause bias in the interpretation of valid samples and affect the reliability of the detection. In addition, the low-depth WGS method may be less sensitive to microdeletions/microduplications of small chromosomal segments and cannot detect triploidy, vanishing twin syndrome, etc. In addition, false positive results are often seen in clinical practice due to the inability to identify common maternal chimerism of low abundance (e.g. 45×) [14].

There may be two methods for detection of chromosomal aneuploidy in fetal cell-free DNA using NGS technology, depending on the experimental principle and data analysis algorithms. In addition to the aforementioned method based on low-depth whole genome sequencing (WGS), there is also a single nucleotide polymorphism (SNP) method based on targeted sequencing [15]. The present disclosure uses a quantitative analysis method based on SNP targeted high-depth sequencing and genotyping (SNP-targeted high-depth sequencing and genotyping) to quantify NIPS, showing its advantages over the WGS method (shown in the table below). The method features the use of maternal genotype information and paternal genotype estimated by frequencies of SNPs in population to construct possible fetal normal or abnormal genotypes caused by chromosome copy number variation. The method features the use of maternal genotype information and paternal genotype estimated by frequencies of SNPs in population to construct possible fetal normal or abnormal genotypes. The probability of each fetal genotype may be calculated by comparing the theoretical predicted value of minor allele fraction (MAF) at each SNP site with the actual measured value in plasma cell-free DNA. Since this method only examines the quantitative variation of MAF of cell-free DNA at each SNP site to derive the possible fetal genotype, it may not require the use of diploid reference chromosomes as in the case of NIPS with WGS, thus simplifying the detection operations and analysis requirements. However, current SNP methods may be based on multiplex PCR, and this amplification technique may be prone to ADO (allelic drop-out) in the analysis of highly fragmented cell-free DNA, thus requiring the simultaneous analysis of approximately 20,000 SNP sites to improve the signal-to-noise ratio for chromosome copy number quantification [14].

The WGS method may obtain sequencing data (reads) of all chromosomes by whole genome sequencing, detect the relative increase or decrease in reads of chromosomes of interest using aneuploidy-specific algorithms, detect the fetal fraction (FF) in cell-free DNA, and calculate the risk probability of abnormal chromosome number (trisomy or monosomy) by reads distribution and quantitative statistics. In contrast, the SNP method may not perform whole chromosome sequencing on all chromosomes, but only quantitatively genotypes a certain number of polymorphic sites in the genome, and calculates FF and the risk probability of aneuploidy by measuring the difference in the contribution of cell-free DNA from different sources (fetal or maternal) to the genotypic signal. For each SNP site, the contribution of the fetal genome (e.g., C/C with C at 100%) influences to some extent the allelic equilibrium in the maternal genome (e.g., C/T with C at 50%). Thus, when the fetal genotype differs from the maternal genotype with FF at 10%, the balanced C in the cell-free DNA in peripheral blood of pregnant women at this allele site is shifted from 50% to 55%. Thus, the SNP method may allow inferring the risk probability of aneuploidy by analyzing thousands at SNP sites in different regions of the genome, based on the equilibrium shifts of their alleles. For both methods, a goal may be to calculate copy number variation from reads of a specific chromosome or genomic region or allelic equilibrium at SNP sites.

Currently, WGS methods (e.g., Illumina) [16] and SNP methods (e.g., Natera) [17] are widely used internationally, while in China, many clinical applications use WGS methods at present [18, 19]. In practical application, the WGS methods have many limitations. Its sensitivity and specificity may be limited by the fetal cell-free DNA fraction, the sensitivity of the detection of microdeletion/microduplication may be low, and it may be difficult to detect twin pregnancies and twin and singleton survival rates, etc. In addition, the WGS method may require more sequencing data and is more costly, whereas the SNP method can avoid unnecessary sequencing reads on non-target chromosomes because it is based on genotyping targeted sequencing. For chromosomal microdeletion/microduplication diseases such as 22q11.2del syndrome with deletion fragments within 0.5-3 Mb, targeted enrichment amplification primers can be designed based on specific chromosomal regions for directed sequencing analysis of chromosomes of interest to achieve higher detection efficiency [20].

### REFERENCES

[1] Ministry of Health of the People's Republic of China, Report on Birth Defects Prevention and Control in China, 2012 is incorporated by reference herein in its entirety.
[2] Nussbaum R L, McInnes R R, Willard H F. Thompson&Thompson Genetics in Medicine. 8th ed. Philadelphia: Saunders/Elsevier; 2015 is incorporated by reference herein in its entirety.
[3] Santorum M1, Wright D2, Syngelaki A1, Karagioti N1, Nicolaides K H. Accuracy of first-trimester combined test in screening for trisomies 21, 18 and 13. Ultrasound Obstet Gynecol. 2017 June; 49(6): 714-720 is incorporated by reference herein in its entirety.
[4] Committee on Practice Bulletins—Obstetrics, Committee on Genetics, and the Society for Maternal-Fetal Medicine. Practice Bulletin No. 163: Screening for Fetal Aneuploidy. Obstet Gynecol. 2016 May; 127(5): e123-37 is incorporated by reference herein in its entirety.
[5] Lo Y M, Corbetta N, Chamberlain P F, Rai V, Sargent I L, Redman C W, et al. Presence of fetal DNA in maternal plasma and serum. Lancet 1997; 350(9076): 485-7 is incorporated by reference herein in its entirety.
[6] Zhang H, Gao Y, Jiang F, Fu M, Yuan Y, et al. Non-invasive prenatal testing for trisomies 21, 18 and 13: clinical experience from 146, 958 pregnancies. Ultrasound Obstet Gynecol. 2015 May; 45(5): 530-8 is incorporated by reference herein in its entirety.
[7] Lo Y M, Tein M S, Lau T K, Haines C J, Leung T N, Poon P M, et al. Quantitative analysis of fetal DNA in maternal plasma and serum: implications for noninvasive prenatal diagnosis. Am J Hum Genet 1998; 62(4): 768-75 is incorporated by reference herein in its entirety.
[8] Lo Y M, Zhang J, Leung T N, Lau T K, Chang A M, Hjelm N M. Rapid clearance of fetal DNA from maternal plasma. Am J Hum Genet 1999; 64(1): 218-24 is incorporated by reference herein in its entirety.
[9] Costa J M, Benachi A, Gautier E. New strategy for prenatal diagnosis of X-linked disorders. N Engl J Med 2002; 346(19): 1502 is incorporated by reference herein in its entirety.
[10] Lo Y M, Hjelm N M, Fidler C, Sargent I L, Murphy M F, Chamberlain P F, et al. Prenatal diagnosis of fetal RhD status by molecular analysis of maternal plasma. N Engl J Med 1998; 339(24): 1734-8 is incorporated by reference herein in its entirety.
[11] Chiu R W, Lau T K, Leung T N, Chow K C, Chui DH, Lo Y M. Prenatal exclusion of beta thalassaemia major by examination of maternal plasma. Lancet 2002; 360(9338): 998-1000 is incorporated by reference herein in its entirety.
[12] Gil M M, Accurti V, Santacruz B, Plana M N, Nicolaides K H. Analysis of cell-free DNA in maternal blood in screening for aneuploidies: updated meta-analysis. Ultrasound Obstet Gynecol 2017; 50: 302-14 is incorporated by reference herein in its entirety.
[13] Srinivasan A, Bianchi D W, Huang H, Sehnert A J, Rava R P. Noninvasive detection of fetal subchromosome abnormalities via deep sequencing of maternal plasma. Am J Hum Genet 2013; 92(2): 167-76 is incorporated by reference herein in its entirety.
[14] Artieri C G, Haverty C, Evans E A, Goldberg J D, Haque I S. Noninvasive prenatal screening at low fetal fraction: comparing whole genome sequencing and single-nucleotide polymorphism methods. Prenat Diagn. 2017 May; 37(5): 482-490 is incorporated by reference herein in its entirety.
[15] Chitty L S, Lo Y M. Noninvasive Prenatal Screening for Genetic Diseases Using Massively Parallel Sequencing of Maternal Plasma DNA. Cold Spring Harb Perspect Med. 2015 Jul. 17; 5(9): a023085 is incorporated by reference herein in its entirety.
[16] Fan H C, Blumenfeld Y J, Chitkara U, Hudgins L, Quake S R. Noninvasive diagnosis of fetal aneuploidy by shotgun sequencing DNA from maternal blood. Proc Natl Acad Sci USA 2008; 105: 16266-71 is incorporated by reference herein in its entirety.
[17] Zimmermann B, Hill M, Gemelos G, Demko Z, Banjevic M, Baner J, et al. Noninvasive prenatal aneuploidy testing of chromosomes 13, 18, 21, X, and Y, using targeted sequencing of polymorphic loci. Prenat Diagn 2012; 32: 1233-41 is incorporated by reference herein in its entirety.
[18] Xu L, Huang H, Lin N, Wang Y, He D, et al. Non-invasive cell-free fetal DNA testing for aneuploidy: multicenter study of 31 515 singleton pregnancies in southeastern China. Ultrasound Obstet Gynecol. 2020 February; 55(2): 242-247 is incorporated by reference herein in its entirety.
[19] Xue Y, Zhao G, Li H, Zhang Q, Lu J, et al. Non-invasive prenatal testing to detect chromosome aneuploidies in 57, 204 pregnancies. Mol Cytogenet. 2019 Jun. 20; 12: 29 is incorporated by reference herein in its entirety.
[20] Martin K, Iyengar S, Kalyan A, Lan C, Simon A L, Stosic M, Kobara K, Ravi H, Truong T, Ryan A, Demko Z P, Benn P. Clin Genet. 2018 February; 93(2): 293-300 is incorporated by reference herein in its entirety.

### SUMMARY

In an aspect, provided herein is a method of analyzing nucleic acid molecules from a biological sample obtained or derived from a subject, comprising: (1) capturing a target nucleic acid molecule obtained or derived from the biological sample using a capture probe, wherein at least a portion of the capture probe is complementary to a target region in a reference genome to which the target nucleic acid molecule aligns, wherein the capture probe is configured to selectively hybridize to a nucleic acid molecule comprising the target region, wherein the target region comprises a single nucleotide polymorphism (SNP) site, wherein the SNP site has a reference allele and an alternative allele among individuals in a reference population, wherein the capture probe comprises a sequence selected from a set of four candidate probe sequences, wherein each of the set of four candidate probe sequences is complementary to the target region and comprises a nucleotide selected from A, T, G, and C, respectively, at a position corresponding to the SNP site, and wherein the sequence of the capture probe is a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele; and (2) analyzing the captured target nucleic acid molecule.

In some embodiments, the target nucleic acid molecule is a cell-free nucleic acid molecule obtained from the biological sample or an amplification product thereof.

In some embodiments, the target nucleic acid molecule is a cellular nucleic acid molecules obtained from the biological sample or an amplification product thereof.

In some embodiments, the method further comprises isolating nucleic acid molecules from the biological sample, wherein the isolated nucleic acid molecules comprise the target nucleic acid molecule.

In some embodiments, the method further comprises amplifying nucleic acid molecules obtained or derived from the biological sample, thereby generating amplification products that comprise the target nucleic acid molecule.

In some embodiments, the pairing kinetics is determined at least in part by measuring a melting temperature for the first hybridizing and the second hybridizing.

In some embodiments, the melting temperature is determined based at least in part on a Nearest Neighbor model.

In some embodiments, the capture probe has a length of 50 to 500 nucleotides (nt). In some embodiments, the capture probe has a length of 100 to 200 nucleotides (nt). In some embodiments, the capture probe has a GC content of 40% to 60%.

In some embodiments, the target region is proximal to or within one or more genes of FGFR3, FGFR2, PTPN11, RAF1, RIT1, SOS1, COL1A1, COL1A2, COL2A1, OTC, or MECP2 in the reference genome.

In some embodiments, the capture probe is free floating in a solution. In some other embodiments, the capture probe is bound to a solid surface.

In some embodiments, the analyzing the captured target nucleic acid molecule comprises sequencing the captured target nucleic acid molecule or an amplified product thereof, thereby obtaining sequence reads corresponding to the target nucleic acid molecule.

In some embodiments, the subject is a pregnant subject carrying a fetus, and wherein the analyzing the captured target nucleic acid molecule further comprises determining a presence or an absence of a chromosomal abnormality, a chromosomal aneuploidy, a chromosomal microdeletion or microduplication, or a monogenic variant in the fetus based at least in part on the sequence reads.

In some embodiments, the chromosomal abnormality comprises maternal trisomy type I, maternal trisomy type II, paternal trisomy type I, paternal trisomy type II, maternal deletion, or paternal deletion. In some embodiments, the SNP site has an allele frequency of 0.2 to 0.8 among the individuals in the reference population. In some embodiments, the SNP site has an allele frequency of 0.3 to 0.7 among the individuals in the reference population.

In some embodiments, the method comprises capturing a plurality of the target nucleic acid molecules that have different nucleic acid sequences using a plurality of the capture probes that have different nucleic acid sequences.

In another aspect, provided herein is a method of designing a capture probe, comprising: (a) determining a target region in a reference genome to which target nucleic acid molecules align, wherein the target region comprises a single nucleotide polymorphism (SNP) site, and wherein the SNP site has a reference allele and an alternative allele among individuals in a reference population; and (b) selecting a sequence for a capture probe for the target region from a set of four candidate probe sequences, wherein each of the set of four candidate sequences is complementary to the target region and comprises a nucleotide selected from A, T, G, and C, respectively, at a position corresponding to the SNP site, and wherein the sequence of the capture probe is a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele.

In another aspect, provided herein is a capture probe having a sequence that is at least 80% identical to a sequence set forth in any one of SEQ ID NOs: 9-13.

In some embodiments, the sequence of the capture probe is at least 85% identical to the sequence set forth in any one of SEQ ID NOs: 9-13. In some embodiments, the sequence of the capture probe is at least 90% identical to the sequence set forth in any one of SEQ ID NOs: 9-13. In some embodiments, the sequence of the capture probe is at least 95% identical to the sequence set forth in any one of SEQ ID NOs: 9-13. In some embodiments, the sequence of the capture probe is at least 99% identical to the sequence set forth in any one of SEQ ID NOs: 9-13. In some embodiments, the sequence of the capture probe is identical to the sequence set forth in any one of SEQ ID NOs: 9-13.

In one aspect, provided herein is a composition comprising a set of different capture probes, each different capture probe of the set of different capture probes having a sequence that is at least 80% identical to a different sequence set forth in SEQ ID NOs: 9-13.

In some embodiments, each different capture probe has a sequence that is at least 85% identical to a different sequence set forth in SEQ ID NOs: 9-13. In some embodiments, each different capture probe has a sequence that is at least 90% identical to a different sequence set forth in SEQ ID NOs: 9-13. In some embodiments, each different capture probe has a sequence that is at least 95% identical to a different sequence set forth in SEQ ID NOs: 9-13. In some embodiments, each different capture probe has a sequence that is at least 99% identical to a different sequence set forth in SEQ ID NOs: 9-13. In some embodiments, each different capture probe has a sequence that is identical to a different sequence set forth in SEQ ID NOs: 9-13.

In another aspect, provided herein is a method of analyzing fetal-derived nucleic acids, comprising: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a plurality of informative single nucleotide polymorphism (SNP) sites on a reference genome of a chromosome, wherein for each of the plurality of informative SNP sites: a first portion of the plurality of sequence reads comprises a reference allele at a position corresponding to the respective informative SNP site, and a second portion of the plurality of sequence reads comprises an alternative allele at the position corresponding to the respective informative SNP site; and (c) determining, based at least in part on the plurality of informative SNP sites, whether the fetus has a chromosomal aneuploidy with one parental meiotic recombination on the chromosome, at least in part by: (i) for each of the plurality of informative SNP sites, determining a difference between a first likelihood of the fetus having disomy (D) and a second likelihood of the fetus having aneuploidy selected from maternal trisomy type I (MI), maternal trisomy type II (MID, paternal trisomy type I (PI), paternal trisomy type II (PII), maternal deletion (LDi), and paternal deletion (LP), respectively; (ii) determining a set of sums of: (1) the differences across a first portion of the plurality of informative SNP sites that are within a first region from a first end of the chromosome to a sliding intermediate point within the chromosome, and (2) the differences across a second portion of the plurality of informative SNP sites that are within a second region from the sliding intermediate point to a second end of the chromosome; (iii) determining a maximum sum of the set of sums; and (iv) determining that the fetus has the chromosomal aneuploidy with one parental meiotic recombination on the chromosome when the maximum sum is within a threshold range.

In some embodiments, the maximum sum of the set of sums is determined according to:

ΔL(H12)=min(Σ₁^{k}(log(LDi)−log(LH1i))+Σₖ₊₁^{M}(log(LDi)−log(LH2i))), and

ΔL(H21)=min(Σ₁^{k}(log(LDi)−log(LH2i))+Σₖ₊₁^{M}(log(LDi)−log(LH1i)))
wherein M is a number of the plurality of informative SNP sites,
wherein k is a varying number from 2 to M−1,
wherein i is an integer from 1 to M,
wherein LDi is a likelihood of the fetus having disomy at an SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with one parental meiotic recombination on the chromosome when any of ΔL(H12) and ΔL(H21) is within the threshold range.

In another aspect, provided herein is a method of analyzing fetal-derived nucleic acids, comprising: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a plurality of informative single nucleotide polymorphism (SNP) sites on a reference genome of a chromosome, wherein for each of the plurality of informative SNP sites: a first portion of the plurality of sequence reads comprises a reference allele at a position corresponding to the respective informative SNP site, and a second portion of the plurality of sequence reads comprises an alternative allele at the position corresponding to the respective informative SNP site; and (c) determining, based at least in part on the plurality of informative SNP sites, whether the fetus has a chromosomal aneuploidy with n parental meiotic recombinations on the chromosome, at least in part by: (i) for each of the plurality of informative SNP sites, determining a difference between a first likelihood of the fetus having disomy (D) and a second likelihood of the fetus having aneuploidy selected from maternal trisomy type I (MI), maternal trisomy type II (MIT), paternal trisomy type I (PI), paternal trisomy type II (PII), maternal deletion (LDi), and paternal deletion (LP), respectively; (ii) determining a set of sums of: (1) the differences across a first portion of the plurality of informative SNP sites that are within a first region from a first end of the chromosome to a first sliding intermediate point within the chromosome, (2) a set of sums of the differences across each one of (n−1) portions of the plurality of informative SNP sites, wherein each one of the (n−1) portions of the plurality of informative SNP sites is within one of (n−1) successive sliding regions within the region from the first sliding intermediate point to a second sliding intermediate point within the chromosome, and (3) the differences across a second portion of the plurality of informative SNP sites that are within a second region from the second slide sliding intermediate point to a second end of the chromosome; (iii) determining a maximum sum of the set of sums; and (iv) determining that the fetus has the chromosomal aneuploidy with n parental meiotic recombinations on the chromosome when the maximum sum is within a threshold range, wherein n is an integer larger than 1.

In some embodiments, the maximum sum of the set of sums is determined according to:

ΔL(H121)=min(Σ₁^{b1}(log(LDi)−log(LH1i))+Σ_{b1}^{b2}(log(LDi)−log(LH2i))+Σ_{b2}^{M}(log(LDi)−log(LH1i))), and Equation 1:

ΔL(H212)=min(Σ₁^{b1}(log(LDi)−log(LH2i))+Σ_{b1}^{b2}(log(LDi)−log(LH1i))+Σ_{b2}^{M}(log(LDi)−log(LH2i)))), Equation 2:
wherein M is a number of the plurality of informative SNP sites,
wherein b1 and b2 are two varying numbers from 2 to M−1, and b1 is smaller than b2,
wherein i is an integer from 1 to M,
wherein LDi is a likelihood of the fetus having disomy at an SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with two parental meiotic recombinations on the chromosome when any of ΔL(H121) and ΔL(H212) is within the threshold range.

In some embodiments, the maximum sum of the set of sums is determined according to:

ΔL(H1212)=min(Σ₁^{b1}(log(LDi)−log(LH1i))+Σ_{b1}^{b2}(log(LDi)−log(LH2i))+Σ_{b2}^{b3}(log(LDi)−log(LH1i))+Σ_{b3}^{M}(log(LDi)−log(LH2i))), Equation 1:

and

ΔL(H2121)=min(Σ₁^{b1}(log(LDi)−log(LH2i))+Σ_{b1}^{b2}(log(LDi)−log(LH1i))+Σ_{b2}^{b3}(log(LDi)−log(LH2i))+Σ_{b3}^{M}(log(LDi)−log(LH1i)))), Equation 2:
wherein M is a number of the plurality of informative SNP sites,
wherein b1, b2, and b3 are four varying numbers from 2 to M−1, and b1 is smaller than b2, and b2 is smaller than b3,
wherein i is an integer from 1 to M,
wherein LDi is a likelihood of the fetus having disomy at an SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with three parental meiotic recombinations on the chromosome when any of ΔL(H1212) and ΔL(H2121) is within the threshold range.

In some embodiments, the maximum sum of the set of sums is determined according to:

ΔL(H12121)=min(Σ₁^{b1}(log(LDi)−log(LH1i))+Σ_{b1}^{b2}(log(LDi)−log(LH2i))+Σ_{b2}^{b3}(log(LDi)−log(LH1i))+Σ_{b3}^{b4}(log(LDi)−log(LH2i))+Σ_{b4}^{M}(log(LDi)−log(LH1i))), Equation 1:

and

ΔL(H21212)=min(Σ₁^{b1}(log(LDi)−log(LH2i))+Σ_{b1}^{b2}(log(LDi)−log(LH1i))+Σ_{b2}^{b3}(log(LDi)−log(LH2i))+Σ_{b3}^{b4}(log(LDi)−log(LH1i))+Σ_{b4}^{M}(log(LDi)−log(LH2i)))), Equation 2:
wherein M is a number of the plurality of informative SNP sites,
wherein b1, b2, b3, and b4 are four varying numbers from 2 to M−1, and b1 is smaller than b2, and b2 is smaller than b3, and b3 is smaller than b4,
wherein i is an integer from 1 to M,
wherein LDi is a likelihood of the fetus having disomy at an i^{th} SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with four parental meiotic recombinations on the chromosome when any of ΔL(H12121), and ΔL(H21212) is within the threshold range.

In another aspect, provided herein is a method of analyzing fetal-derived nucleic acids, comprising: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a plurality of informative single nucleotide polymorphism (SNP) sites on a reference genome of a chromosome, wherein for each of the plurality of informative SNP sites: a first portion of the plurality of sequence reads comprises a reference allele at a position corresponding to the respective informative SNP site, and a second portion of the plurality of sequence reads comprises an alternative allele at the position corresponding to the respective informative SNP site; and (c) determining, based at least in part on the plurality of informative SNP sites, whether the fetus has a chromosomal microdeletion or microduplication on the chromosome, at least in part by: (i) for each of the plurality of informative SNP sites, determining a difference between a first likelihood of the fetus having disomy (D) and a second likelihood of the fetus having aneuploidy selected from maternal trisomy type I (MI), maternal trisomy type II (MII), paternal trisomy type I (PI), paternal trisomy type II (PII), maternal deletion (LDi), and paternal deletion (LP), respectively; (ii) determining a set of sums of the differences across a portion of the plurality of informative SNP sites that are within a sliding window of a varying length along the chromosome; (iii) determining a maximum sum of the set of sums; and (iv) determining that the fetus has the chromosomal microdeletion or microduplication when the maximum sum is within a threshold range.

In some embodiments, the maximum sum of the differences is determined according to:

ΔL=min(Σb₁^{b2}(log(LDi)−log(LHi))),
wherein M is a number of the plurality of informative SNP sites,
wherein b1 and b2 are two varying numbers from 1 to M, and b1 is smaller than b2,
wherein i is an integer from 1 to M,
wherein LDi is a likelihood of the fetus having disomy at an SNP site among the plurality of informative SNP sites,
wherein LH1i is a likelihood of the fetus being H at the i^{th} SNP site among the plurality of informative SNP sites,
wherein Hϵ{MI, MII, PI, PII, LM, LP}, and
wherein the fetus is determined to have the chromosomal microdeletion or microduplication on the chromosome when ΔL is within the threshold range.

In some embodiments, the first likelihood of the fetus having disomy (D) and the second likelihood of the fetus having chromosomal aneuploidy at the respective informative SNP site are determined using a beta-binominal distribution.

In some embodiments, the first likelihood of the fetus having disomy (D) and the second likelihood of the fetus having chromosomal aneuploidy at the respective informative SNP site are determined according to:

log(p(NAi,N,pAi,H))=log(Σₖπk Beta−Binom(pAi,N,α,β)),
wherein M is a number of the plurality of informative SNP sites,
wherein i is an integer from 1 to M,
wherein N is a sequencing depth of the plurality of sequence reads at an SNP site among the plurality of informative SNP sites,
wherein pAi is an expected value of a percentage of sequence reads having an alternative allele at the i^{th} SNP site from next generation sequencing (NGS) given an assumption that the fetus has different euploid and aneuploid states,
wherein α is a pre-determined discrete parameter between 1000 to 5000;
wherein β=α/pAi−α,
wherein πk is a multinomial factor for a karyotype selected from a set of k different potential karyotypes of the fetus and is determined according to:

$$π k = {\sum }_{P A T k} p ( FET ) \times p ( P A T k )$$

wherein PATkϵ{AA, AB, BB}, and p(PATk) is determined using the Hardy-Weinberg equation, according to:

p(AA)=p×p

p(AB)=2×p×(1−p)

p(BB)=(1−p)×(1−p)
wherein p denotes frequency of the alternative allele at the SNP site in a reference population, and
wherein p(FET) is a probability of a specific fetal genotype in different euploid and aneuploid states when a familial trio is analyzed following Mendelian inheritance principles.

In some embodiments, the threshold range is set forth in Table 3 for a karyotype of MI, MII, PI, PII, LM, and LP, respectively.

In another aspect, provided herein is a method of analyzing fetal-derived nucleic acids, comprising: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a variant site on a reference genome, wherein a portion of the plurality of sequence reads has an alternative allele at a position corresponding to the variant site, and wherein the pregnant subject is homozygous for a reference allele at the position corresponding to the variant site; and (c) determining whether the fetus has dominant monogenic variation at the variant site at least in part by: (i) determining a likelihood of the alternative allele being a paternally inherited or de novo fetal mutation at least in part by determining a difference between a first likelihood of the fetal-derived nucleic acid molecules having the alternative allele and a second likelihood of the reference allele being derived from systemic noise; and (ii) determining that the fetus has the dominant monogenic variation at the variant site when the difference is within a threshold range.

In some embodiments, the likelihood of the alternative allele being the paternally inherited or de novo fetal mutation is determined according to:

ΔL=log(beta−binom(ff/2,N,α,β1))−log(beta−binom(e,N,α,β2)),
wherein N is a sequencing depth of the plurality of sequence reads at the variant site,
wherein ff is a fraction of the fetal-derived nucleic acid molecules in the nucleic acid molecules (fetal fraction),
wherein α is a pre-determined discrete parameter from 1000 to 5000;
wherein β1=2×α/ff−α,
wherein e is a systematic error rate at the variant site, given by a ratio of mutant genotypes detected at the variant site in negative test samples that do not have the mutant genotypes in fetal nucleic acid molecules,
wherein β2=α/e−α,
and wherein the fetus is determined to have the dominant monogenic variation when ΔL is greater than 1.

In some embodiments, ff is determined at least in part by: (i) identifying, based at least in part on the plurality of sequence reads, a plurality of informative SNP sites on a reference genome, wherein α portion of the plurality of sequence reads has a respective alternative allele (“A” allele) at a position corresponding to the respective informative SNP site, and wherein the pregnant subject is homozygous for a respective reference allele (“B” allele) at the position corresponding to the respective informative SNP site; (ii) for each of the plurality of informative SNP sites, determining a fraction of sequence reads that are homozygous for the respective alternative allele (ffAAᵢ) and a fraction of sequence reads that are homozygous for the respective reference allele (ffAAᵢ); and (iii) determining ff according to:

ff=(ffAA+ffBB)/2,
wherein ffAA is a median value of ffAAᵢ across the plurality of informative SNP sites, and ffBB is a median value of ffBBi across the plurality of informative SNP sites.

In some embodiments, α is determined based at least in part on systemic noise of a sequencing procedure that generates the plurality of sequence reads. In some embodiments, α is determined based at least in part on an empirically measured value of a known paternal allele in fetal-derived nucleic acid molecules at the variant site from a positive test sample. In some embodiments, α is about 1000, 2000, 3000, 4000, or 5000.

In some embodiments, the method further comprises capturing, using a capture probe, the nucleic acid molecules from the biological sample that comprise the target region, and sequencing at least a portion of the captured nucleic acid molecules or amplified products thereof. In some embodiments, at least a portion of the capture probe is complementary to the target region, wherein the SNP site has a reference allele and an alternative allele among individuals in a reference population, wherein the capture probe comprises a sequence selected from a set of four candidate probe sequences, wherein each of the set of four candidate probe sequences is complementary to the target region and comprises a nucleotide selected from A, T, G, and C, respectively, at a position corresponding to the SNP site, and wherein the sequence of the capture probe is a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele.

In some embodiments, the nucleic acid molecules obtained or derived from the biological sample comprise cell-free nucleic acid molecules. In some embodiments, the nucleic acid molecules obtained or derived from the biological sample comprise cell-free nucleic acid molecules and cellular nucleic acid molecules.

In another aspect, provided herein is a computer system, comprising one or more processors; and a non-transitory computer readable medium comprising instructions operable, when executed by the one or more computer processors, to cause the computer system to perform any of the methods disclosed herein.

In another aspect, provided herein is a non-transitory computer-readable storage medium comprising instructions operable, when executed by one or more processors of a computer system, to cause the computer system to perform any of the methods disclosed herein.

In another aspect, provided herein is a system configured to perform any of the methods disclosed herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a comparison of enrichment degrees of target regions before and after capture. For a DNA fragment in a non-capture region, the enrichment degrees before and after hybridization capture are not changed. However, for a DNA fragment in a capture region, the enrichment degree after hybridization capture is 10 times greater than that before hybridization capture, which satisfies quality control requirement.

FIG. 2 shows that capture efficiencies of a target region with a duration of hybridization capture for 4 h or 16 h are not obviously changed.

FIG. 3 shows quantitative analysis on enrichment degree comparison of a target region before and after capture.

FIG. 4a and FIG. 4b show quantitative analysis on enrichment degree comparison of a target region before and after capture.

FIG. 5 shows a result that mutant genes obtained by COATE method improve capture homogeneity of alleles.

FIG. 6 shows a result that COATE method reduces sampling bias.

FIG. 7a and FIG. 7b respectively show a fluctuation range of experiment error measured by CAF in a sample and a result of an average CAF value of heterozygote mutation of a sample.

FIG. 8 shows a result of a fluctuation range of detection error for NGS sequencing of germ-line heterozygote CAF.

FIG. 9 shows a comparison result of SNP-based fetal DNA fraction calculation and Y-chromosome calculation (N=128).

FIG. 10a, FIG. 10b, FIG. 10c, and FIG. 10d respectively show the probability values of L(D)-L(H), Hϵ{MI, MIT, PI, PII} of chromosomes 13, 18, and 21 in 203 negative samples, in which the L(D)-L(H) difference values of 202 samples are greater than −10, and the difference value of one negative sample is less than −10. The conclusion is that a false positive rate is about 0.5% if the negative threshold is set as −10.

FIG. 11a and FIG. 11b respectively show a relationship between the probability values of L (D)-L(MI) of chromosomes 13, 18 and 21 in positive reference and mixing ratios of the positive reference, and the part in a small block in FIG. 11a is amplified and shown in FIG. 11b. When the mixing ratio of the positive reference is larger than 4%, the value of L(D)-L(MI) is less than −10.

FIG. 12 shows a relationship between the probability values of L(D)-L(MI) of chromosomes 13, 18, and 21 in positive maternal plasma and fetal fraction. When the fetal fraction is larger than 4%, the values of L(D)-L(MI) of all the positive samples are less than −10.

FIG. 13a and FIG. 13b respectively show L(D)-L(MI) and L(D)-L(MII) values, moving average lines and their accumulation curves of a chromosome 21 abnormal sample at different SNP sites; FIG. 13c and FIG. 13d respectively show L(D)-L(MI) and L(D)-L(MII) values, moving average lines and their accumulation curves of a chromosome 13 abnormal sample at different SNP sites.

FIG. 14 shows values and moving average lines of L(D)-L(LDi) and L(D)-L(LP) of chromosome 22 at different SNP sites.

FIG. 15 shows a computer system that can be programmed or otherwise configured to implement methods provided herein.

FIG. 16 shows an example of a diagram of the methods and systems as disclosed herein.

### DETAILED DESCRIPTION

The present disclosure generally relates to methods, kits, computer-readable media, and systems for analysis of nucleic acid molecules, for instance, for detection of chromosomal aneuploidy and/or monogenic variation. In some cases, the present disclosure relates to non-invasive prenatal detection by analyzing biological sample from a pregnant subject. In some cases, the present disclosure relates to analysis of cell-free nucleic acid molecules, e.g., cell-free DNA, in biological samples, such as blood plasma.

In some embodiments, provided herein is a method of analyzing nucleic acid molecules from a biological sample obtained or derived from a subject, for instance, a method useful for coordinative allele-aware target enrichment (COATE) of target nucleic acid molecules obtained or derived from a biological sample. In some embodiments, the method comprises: (1) capturing a target nucleic acid molecule obtained or derived from the biological sample using a capture probe, wherein at least a portion of the capture probe is complementary to a target region in a reference genome to which the target nucleic acid molecule aligns, wherein the capture probe is configured to selectively hybridize to a nucleic acid molecule comprising the target region, wherein the target region comprises a single nucleotide polymorphism (SNP) site, wherein the SNP site has a reference allele and an alternative allele among individuals in a reference population, wherein the capture probe comprises a sequence selected from a set of four candidate probe sequences, wherein each of the set of four candidate probe sequences is complementary to the target region and comprises a nucleotide selected from A, T, G, and C, respectively, at a position corresponding to the SNP site, and wherein the sequence of the capture probe is a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele; and (2) analyzing the captured target nucleic acid molecule. Without wishing to be bound by a certain theory, the method disclosed herein relates to reducing capturing bias by reducing the difference in pairing kinetics between the hybridization of the capture probe with different target nucleic acid molecules that have different alleles are SNP site(s).

In some embodiments, the method disclosed herein further comprises isolating nucleic acid molecules from the biological sample, wherein the isolated nucleic acid molecules comprise the target nucleic acid molecule. In some embodiments, the method further comprises amplifying nucleic acid molecules obtained or derived from the biological sample, thereby generating amplification products that comprise the target nucleic acid molecule. In some embodiments, the pairing kinetics is determined at least in part by measuring a melting temperature for the first hybridizing and the second hybridizing.

In some embodiments, the melting temperature (Tm) is determined based at least in part on a Nearest Neighbor model. For instance, the melting temperature Tm is calculated according to the following equation:

$${T}_{m} = \frac{Δ H}{Δ S + R \times ln {C}_{T}} + 1 6.6 log [ {Na}^{+} ]$$

ΔH represents the sum of standard enthalpy changes for all adjacent base pairs, ΔS represents the sum of standard entropy changes for all adjacent base pairs, R is the molar gas constant, CT represents the concentration of the primers, and [Na+] represents the concentration of monovalent sodium ions in solution.

In some embodiments, the capture probe has a length of 50 to 500 nucleotides (nt), for instance, 50 to 450, 50 to 400, 50 to 350, 50 to 300, 50 to 250, 50 to 200, 50 to 150, 50 to 100, 100 to 500, 100 to 450, 100 to 400, 100 to 350, 100 to 300, 100 to 250, 100 to 200, 100 to 150, 150 to 500, 150 to 450, 150 to 400, 150 to 350, 150 to 300, 150 to 250, 150 to 200, 200 to 500, 200 to 450, 200 to 400, 200 to 350, 200 to 300, 200 to 250, 250 to 500, 250 to 450, 250 to 400, 250 to 350, 250 to 300, 300 to 500, 300 to 450, 300 to 400, 300 to 350, 350 to 500, 350 to 450, 350 to 400, 400 to 500, or 400 to 450 nt. In some embodiments, the capture probe has a length of 100 to 200 nucleotides (nt). In some embodiments, the capture probe has a GC content of 40% to 60%, for instance, 40% to 50%, 51%, 52%, 53%, 54%, 55%, 56%, 57%, 58%, 59%, or 60%, or 45% to 50%, 51%, 52%, 53%, 54%, 55%, 56%, 57%, 58%, 59%, or 60%, or 50% to 51%, 52%, 53%, 54%, 55%, 56%, 57%, 58%, 59%, or 60%.

In some embodiments, the method disclosed herein is applicable to analysis of target nucleic acid molecules that map to the target region that is proximal to or within one or more genes of FGFR3, FGFR2, PTPN11, RAF1, RIT1, SOS1, COL1A1, COL1A2, COL2A1, OTC, or MECP2 in a reference genome. In some embodiments, the SNP site has an allele frequency of 0.2 to 0.8 among the individuals in the reference population. In some embodiments, the SNP site has an allele frequency of 0.3 to 0.7 among the individuals in the reference population.

In some embodiments, the method comprises capturing a plurality of the target nucleic acid molecules that have different nucleic acid sequences using a plurality of the capture probes that have different nucleic acid sequences. For instance, the method may involve use of at least 20, 50, 100, 150, 200, 250, 300, 350, 400, 450, 500, 550, 600, 650, 700, 750, 800, 850, 900, 950, 1000, 1200, 1400, 1500, 1600, 1800, 2000, 2400, 2500, 2800, 3000, 3500, 4000, 4500, 5000, 7000, 7500, 8000, 9000, 10,000, or more different capture probes. In some embodiments, the method may involve a plurality of capture probes that cover (e. g., map to a region in a reference genome that covers) at least 10, 20, 50, 100, 150, 200, 250, 300, 350, 400, 450, 500, 550, 600, 650, 700, 750, 800, 850, 900, 950, 1000, 1200, 1400, 1500, 1600, 1800, 2000, 2400, 2500, 2800, 3000, 3500, 4000, 4500, 5000, 7000, 7500, 8000, 9000, 10,000, 15,000, 20,000, 30,000, 50,000, 75,000, 100,000, or more SNP sites.

In some embodiments, the capture probe used in the method, composition, kit, or system disclosed herein is free floating in a solution. In some embodiments, the capture probe used in the method, composition, kit, or system disclosed herein is bound to a solid surface, for instance, bound to a bead.

In some embodiments, the method disclosed herein is applicable to preparation of nucleic acid molecules for sequencing. In some embodiments, the analyzing operation in the method disclosed herein comprises sequencing the captured target nucleic acid molecule or an amplified product thereof, thereby obtaining sequence reads corresponding to the target nucleic acid molecule.

In some embodiments, the subject is a pregnant subject carrying a fetus, and wherein the analyzing the captured target nucleic acid molecule further comprises determining a presence or an absence of a chromosomal abnormality, a chromosomal aneuploidy, a chromosomal microdeletion or microduplication, or a monogenic variant in the fetus based at least in part on the sequence reads.

In some embodiments, the chromosomal abnormality that the method disclosed herein may be used to detect comprises maternal trisomy type I, maternal trisomy type II, paternal trisomy type I, paternal trisomy type II, maternal deletion, or paternal deletion.

In some aspects, provided herein is a method of designing a capture probe, comprising: (a) determining a target region in a reference genome to which target nucleic acid molecules align, wherein the target region comprises a single nucleotide polymorphism (SNP) site, and wherein the SNP site has a reference allele and an alternative allele among individuals in a reference population; and (b) selecting a sequence for a capture probe for the target region from a set of four candidate probe sequences, wherein each of the set of four candidate sequences is complementary to the target region and comprises a nucleotide selected from A, T, G, and C, respectively, at a position corresponding to the SNP site, and wherein the sequence of the capture probe is a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele.

In some aspects, provided herein is a capture probe that covers a target region is proximal to or within one or more genes of FGFR3, FGFR2, PTPN11, RAF1, RIT1, SOS1, COL1A1, COL1A2, COL2A1, OTC, or MECP2 in a reference genome. In some aspects, provided herein is a capture probe having a sequence that is at least 80% identical to a sequence set forth in any one of SEQ ID NOs: 9-13.

In some embodiments, the sequence of the capture probe is at least 85% identical to the sequence set forth in any one of SEQ ID NOs: 9-13. In some embodiments, the sequence of the capture probe is at least 90% identical to the sequence set forth in any one of SEQ ID NOs: 9-13. In some embodiments, the sequence of the capture probe is at least 95% identical to the sequence set forth in any one of SEQ ID NOs: 9-13. In some embodiments, the sequence of the capture probe is at least 99% identical to the sequence set forth in any one of SEQ ID NOs: 9-13. In some embodiments, the sequence of the capture probe is identical to the sequence set forth in any one of SEQ ID NOs: 9-13. In some embodiments, provided herein is a capture probe that is at least 80%, 85%, 90%, 95%, 96%, 97%, 98%, or 99%, or 100% identical to the sequence set forth in SEQ ID NO: 9. In some embodiments, provided herein is a capture probe that is at least 80%, 85%, 90%, 95%, 96%, 97%, 98%, or 99%, or 100% identical to the sequence set forth in SEQ ID NO: 10. In some embodiments, provided herein is a capture probe that is at least 80%, 85%, 90%, 95%, 96%, 97%, 98%, or 99%, or 100% identical to the sequence set forth in SEQ ID NO: 11. In some embodiments, provided herein is a capture probe that is at least 80%, 85%, 90%, 95%, 96%, 97%, 98%, or 99%, or 100% identical to the sequence set forth in SEQ ID NO: 12. In some embodiments, provided herein is a capture probe that is at least 80%, 85%, 90%, 95%, 96%, 97%, 98%, or 99%, or 100% identical to the sequence set forth in SEQ ID NO: 13.

In some aspects, provided herein is a composition comprising a set of different capture probes, each different capture probe of the set of different capture probes having a sequence that is at least 80% identical to a different sequence set forth in SEQ ID NOs: 9-13. In some embodiments of the composition, each different capture probe has a sequence that is at least 85% identical to a different sequence set forth in SEQ ID NOs: 9-13. In some embodiments of the composition, each different capture probe has a sequence that is at least 90% identical to a different sequence set forth in SEQ ID NOs: 9-13. In some embodiments of the composition, each different capture probe has a sequence that is at least 95% identical to a different sequence set forth in SEQ ID NOs: 9-13. In some embodiments of the composition, each different capture probe has a sequence that is at least 99% identical to a different sequence set forth in SEQ ID NOs: 9-13. In some embodiments of the composition, each different capture probe has a sequence that is identical to a different sequence set forth in SEQ ID NOs: 9-13.

In some aspects, provided herein is a method of analyzing fetal-derived nucleic acids, for instance, a method useful for detecting chromosomal aneuploidy. In some embodiments, the method is useful for detecting chromosomal aneuploidy with at least one, two, three, four, five, six, seven, eight, nine, or even more parental meiotic chromosomal recombinations.

In some embodiments, the method comprises: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a plurality of informative single nucleotide polymorphism (SNP) sites on a reference genome of a chromosome, wherein for each of the plurality of informative SNP sites: a first portion of the plurality of sequence reads comprises a reference allele at a position corresponding to the respective informative SNP site, and a second portion of the plurality of sequence reads comprises an alternative allele at the position corresponding to the respective informative SNP site; and (c) determining, based at least in part on the plurality of informative SNP sites, whether the fetus has a chromosomal aneuploidy with one parental meiotic recombination on the chromosome, at least in part by: (i) for each of the plurality of informative SNP sites, determining a difference between a first likelihood of the fetus having disomy (D) and a second likelihood of the fetus having aneuploidy selected from maternal trisomy type I (MI), maternal trisomy type II (MID, paternal trisomy type I (PI), paternal trisomy type II (PII), maternal deletion (LDi), and paternal deletion (LP), respectively; (ii) determining a set of sums of: (1) the differences across a first portion of the plurality of informative SNP sites that are within a first region from a first end of the chromosome to a sliding intermediate point within the chromosome, and (2) the differences across a second portion of the plurality of informative SNP sites that are within a second region from the sliding intermediate point to a second end of the chromosome; (iii) determining a maximum sum of the set of sums; and (iv) determining that the fetus has the chromosomal aneuploidy with one parental meiotic recombination on the chromosome when the maximum sum is within a threshold range.

In some embodiments, for determining whether the fetus has chromosomal aneuploidy with one parental meiotic recombination, the maximum sum of the set of sums is determined according to:

ΔL(H12)=min(Σ₁^{k}(log(LDi)−log(LH1i))+Σₖ₊₁^{M}(log(LDi)−log(LH2i))), and

ΔL(H21)=min(Σ₁^{k}(log(LDi)−log(LH2i))+Σₖ₊₁^{M}(log(LDi)−log(LH1i)))
wherein M is a number of the plurality of informative SNP sites,
wherein k is a varying number from 2 to M−1,
wherein i is an integer from 1 to M,
wherein LDi is a likelihood of the fetus having disomy at an SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with one parental meiotic recombination on the chromosome when any of ΔL(H12) and ΔL(H21) is within the threshold range.

In some embodiments, the method disclosed herein is useful for detecting whether the fetus has chromosomal aneuploidy with two or more parental meiotic recombinations. In some embodiments, the method comprises: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a plurality of informative single nucleotide polymorphism (SNP) sites on a reference genome of a chromosome, wherein for each of the plurality of informative SNP sites: a first portion of the plurality of sequence reads comprises a reference allele at a position corresponding to the respective informative SNP site, and a second portion of the plurality of sequence reads comprises an alternative allele at the position corresponding to the respective informative SNP site; and (c) determining, based at least in part on the plurality of informative SNP sites, whether the fetus has a chromosomal aneuploidy with n parental meiotic recombinations on the chromosome, at least in part by: (i) for each of the plurality of informative SNP sites, determining a difference between a first likelihood of the fetus having disomy (D) and a second likelihood of the fetus having aneuploidy selected from maternal trisomy type I (MI), maternal trisomy type II (MIT), paternal trisomy type I (PI), paternal trisomy type II (PII), maternal deletion (LDi), and paternal deletion (LP), respectively; (ii) determining a set of sums of: (1) the differences across a first portion of the plurality of informative SNP sites that are within a first region from a first end of the chromosome to a first sliding intermediate point within the chromosome, (2) a set of sums of the differences across each one of (n−1) portions of the plurality of informative SNP sites, wherein each one of the (n−1) portions of the plurality of informative SNP sites is within one of (n−1) successive sliding regions within the region from the first sliding intermediate point to a second sliding intermediate point within the chromosome, and (3) the differences across a second portion of the plurality of informative SNP sites that are within a second region from the second slide sliding intermediate point to a second end of the chromosome; (iii) determining a maximum sum of the set of sums; and (iv) determining that the fetus has the chromosomal aneuploidy with n parental meiotic recombinations on the chromosome when the maximum sum is within a threshold range, wherein n is an integer larger than 1.

In some embodiments, for determining whether the fetus has chromosomal aneuploidy with two parental meiotic recombinations, the maximum sum of the set of sums is determined according to:

ΔL(H121)=min(Σ₁^{b1}(log(LDi)−log(LH1i))+Σ_{b1}^{b2}(log(LDi)−log(LH2i))+Σ_{b2}^{M}(log(LDi)−log(LH1i))), and Equation 1:

ΔL(H212)=min(Σ₁^{b1}(log(LDi)−log(LH2i))+Σ_{b1}^{b2}(log(LDi)−log(LH1i))+Σ_{b2}^{M}(log(LDi)−log(LH2i)))), Equation 2:
wherein M is a number of the plurality of informative SNP sites,
wherein b1 and b2 are two varying numbers from 2 to M−1, and b1 is smaller than b2,
wherein i is an integer from 1 to M,
wherein LDi is a likelihood of the fetus having disomy at an i^{th} SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with two parental meiotic recombinations on the chromosome when any of ΔL(H121) and ΔL(H212) is within the threshold range.

In some embodiments, for determining whether the fetus has chromosomal aneuploidy with three parental meiotic recombinations, the maximum sum of the set of sums is determined according to:

ΔL(H1212)=min(Σ₁^{b1}(log(LDi)−log(LH1i))+Σ_{b1}^{b2}(log(LDi)−log(LH2i))+Σ_{b2}^{b3}(log(LDi)−log(LH1i))+Σ_{b3}^{M}(log(LDi)−log(LH2i))), Equation 1:

and

ΔL(H2121)=min(Σ₁^{b1}(log(LDi)−log(LH2i))+Σ_{b1}^{b2}(log(LDi)−log(LH1i))+Σ_{b2}^{b3}(log(LDi)−log(LH2i))+Σ_{b3}^{M}(log(LDi)−log(LH1i)))), Equation 2:
wherein M is a number of the plurality of informative SNP sites,
wherein b1, b2, and b3 are four varying numbers from 2 to M−1, and b1 is smaller than b2, and b2 is smaller than b3,
wherein i is an integer from 1 to M,
wherein LDi is a likelihood of the fetus having disomy at an i^{th} SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with three parental meiotic recombinations on the chromosome when any of ΔL(H1212) and ΔL(H2121) is within the threshold range.

In some embodiments, for determining whether the fetus has chromosomal aneuploidy with four parental meiotic recombinations, the maximum sum of the set of sums is determined according to:

ΔL(H12121)=min(Σ₁^{b1}(log(LDi)−log(LH1i))+Σ_{b1}^{b2}(log(LDi)−log(LH2i))+Σ_{b2}^{b3}(log(LDi)−log(LH1i))+Σ_{b3}^{b4}(log(LDi)−log(LH2i))+Σ_{b4}^{M}(log(LDi)−log(LH1i))), Equation 1:

and

ΔL(H21212)=min(Σ₁^{b1}(log(LDi)−log(LH2i))+Σ_{b1}^{b2}(log(LDi)−log(LH1i))+Σ_{b2}^{b3}(log(LDi)−log(LH2i))+Σ_{b3}^{b4}(log(LDi)−log(LH1i))+Σ_{b4}^{M}(log(LDi)−log(LH2i)))), Equation 2:
wherein M is a number of the plurality of informative SNP sites,
wherein b1, b2, b3, and b4 are four varying numbers from 2 to M−1, and b1 is smaller than b2, and b2 is smaller than b3, and b3 is smaller than b4,
wherein i is an integer from 1 to M,
wherein LDi is a likelihood of the fetus having disomy at an SNP site among the plurality of informative SNP sites,
wherein LH1i and LH2i are likelihoods of the fetus being H1 or H2, respectively, at the i^{th} SNP site among the plurality of informative SNP sites,
wherein H1, H2ϵ{MI, MII, PI, PII}, and
wherein the fetus is determined to have the chromosomal aneuploidy with four parental meiotic recombinations on the chromosome when any of ΔL(H12121), and ΔL(H21212) is within the threshold range.

In some embodiments, for determining whether the fetus has chromosomal aneuploidy, the method involves taking assumption that there has been zero, one, two, three, four, five, six, seven, eight, nine, or even parental meiotic recombinations, and calculating the maximum sum of the set of sums based on different assumptions, and determining whether the fetus has chromosomal aneuploidy based, at least in part, on the maximum sum of the set of sums. For instance, if the maximum sum of the set of sums is within the threshold range under a given assumption that there has been a given number of parental meiotic recombinations, then the fetus is determined to have chromosomal aneuploidy with the given number of parental meiotic recombinations.

Without wishing to be bound by a certain theory, the method disclosed herein that involves assumptions of parental meiotic chromosomal recombination(s) increase the sensitivity of the detection of chromosomal aneuploidy, e.g., reducing the false negative rate, as compared to detection methods (e.g., maximum likelihood method) that do not consider or take assumption on parental meiotic chromosomal recombinations.

In some aspects, provided herein is a method of analyzing fetal-derived nucleic acids, for instance, a method useful for detecting chromosomal microdeletion and/or microduplication. In some embodiments, the method comprises: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a plurality of informative single nucleotide polymorphism (SNP) sites on a reference genome of a chromosome, wherein for each of the plurality of informative SNP sites: a first portion of the plurality of sequence reads comprises a reference allele at a position corresponding to the respective informative SNP site, and a second portion of the plurality of sequence reads comprises an alternative allele at the position corresponding to the respective informative SNP site; and (c) determining, based at least in part on the plurality of informative SNP sites, whether the fetus has a chromosomal microdeletion or microduplication on the chromosome, at least in part by: (i) for each of the plurality of informative SNP sites, determining a difference between a first likelihood of the fetus having disomy (D) and a second likelihood of the fetus having aneuploidy selected from maternal trisomy type I (MI), maternal trisomy type II (MID, paternal trisomy type I (PI), paternal trisomy type II (PII), maternal deletion (LDi), and paternal deletion (LP), respectively; (ii) determining a set of sums of the differences across a portion of the plurality of informative SNP sites that are within a sliding window of a varying length along the chromosome (iii) determining a maximum sum of the set of sums; and (iv) determining that the fetus has the chromosomal microdeletion or microduplication when the maximum sum is within a threshold range.

In some embodiments, for detection of chromosomal microdeletion and/or microduplication, the maximum sum of the differences is determined according to:

ΔL=min(Σb₁^{b2}(log(LDi)−log(LHi))),
wherein M is a number of the plurality of informative SNP sites,
wherein b1 and b2 are two varying numbers from 1 to M, and b1 is smaller than b2,
wherein i is an integer from 1 to M,
wherein LDi is a likelihood of the fetus having disomy at an SNP site among the plurality of informative SNP sites,
wherein LH1i is a likelihood of the fetus being H at the i^{th} SNP site among the plurality of informative SNP sites,
wherein Hϵ{MI, MII, PI, PII, LM, LP}, and
wherein the fetus is determined to have the chromosomal microdeletion or microduplication on the chromosome when ΔL is within the threshold range.

In some embodiments of the method disclosed herein, the first likelihood of the fetus having disomy (D) and the second likelihood of the fetus having chromosomal aneuploidy at the respective informative SNP site are determined using a beta-binominal distribution. In some embodiments, the first likelihood of the fetus having disomy (D) and the second likelihood of the fetus having chromosomal aneuploidy at the respective informative SNP site are determined according to:

log(p(NAi,N,pAi,H))=log(Σₖπk Beta−Binom(pAi,N,α,β)),
wherein M is a number of the plurality of informative SNP sites,
wherein i is an integer from 1 to M,
wherein N is a sequencing depth of the plurality of sequence reads at an SNP site among the plurality of informative SNP sites,
wherein pAi is an expected value of a percentage of sequence reads having an alternative allele at the i^{th} SNP site from next generation sequencing (NGS) given an assumption that the fetus has different euploid and aneuploid states,
wherein α is a pre-determined discrete parameter between 1000 to 5000;
wherein β=α/pAi−α,
wherein πk is a multinomial factor for a karyotype selected from a set of k different potential karyotypes of the fetus and is determined according to:

$$π k = {\sum }_{P A T k} p ( FET ) \times p ( P A T k )$$

wherein PATk ϵ{AA, AB, BB}, and p(PATk) is determined using the Hardy-Weinberg equation, according to:

p(AA)=p×p

p(AB)=2×p×(1−p)

p(BB)=(1−p)×(1−p)
wherein p denotes frequency of the alternative allele at the SNP site in a reference population, and
wherein p(FET) is a probability of a specific fetal genotype in different euploid and aneuploid states when a familial trio is analyzed following Mendelian inheritance principles.

In some embodiments of the method disclosed herein, the threshold range for detecting chromosomal aneuploidy, or chromosomal microdeletion or microduplication, is set forth in Table 3 for a karyotype of MI, MII, PI, PII, LM, and LP, respectively.

In some aspects, provided herein is a method of analyzing fetal-derived nucleic acids, for instance, a method useful for detecting dominant monogenic variation in a fetus. In some embodiments, the method comprises: (a) obtaining a plurality of sequence reads of nucleic acid molecules obtained or derived from a biological sample from a pregnant subject carrying a fetus, wherein the nucleic acid molecules comprise maternal-derived nucleic acid molecules from the pregnant subject and fetal-derived nucleic acid molecules from the fetus; (b) identifying, based at least in part on the plurality of sequence reads, a variant site on a reference genome, wherein a portion of the plurality of sequence reads has an alternative allele at a position corresponding to the variant site, and wherein the pregnant subject is homozygous for a reference allele at the position corresponding to the variant site; and (c) determining whether the fetus has dominant monogenic variation at the variant site at least in part by: (i) determining a likelihood of the alternative allele being a paternally inherited or de novo fetal mutation at least in part by determining a difference between a first likelihood of the fetal-derived nucleic acid molecules having the alternative allele and a second likelihood of the reference allele being derived from systemic noise; and (ii) determining that the fetus has the dominant monogenic variation at the variant site when the difference is within a threshold range.

In some embodiments, for detecting dominant monogenic variation, the likelihood of the alternative allele being the paternally inherited or de novo fetal mutation is determined according to:

ΔL=log(beta−binom(ff/2,N,α,β1))−log(beta−binom(e,N,α,β2)),
wherein N is a sequencing depth of the plurality of sequence reads at the variant site,
wherein ff is a fraction of the fetal-derived nucleic acid molecules in the nucleic acid molecules (fetal fraction),
wherein α is a pre-determined discrete parameter from 1000 to 5000;
wherein β1=2×α/ff−α,
wherein e is a systematic error rate at the variant site, given by a ratio of mutant genotypes detected at the variant site in negative test samples that do not have the mutant genotypes in fetal nucleic acid molecules,
wherein β2=α/e−α,
and wherein the fetus is determined to have the dominant monogenic variation when ΔL is greater than 1.

In some embodiments, fetal fraction as disclosed herein (ff) is determined at least in part by: (i) identifying, based at least in part on the plurality of sequence reads, a plurality of informative SNP sites on a reference genome, wherein α portion of the plurality of sequence reads has a respective alternative allele (“A” allele) at a position corresponding to the respective informative SNP site, and wherein the pregnant subject is homozygous for a respective reference allele (“B” allele) at the position corresponding to the respective informative SNP site; (ii) for each of the plurality of informative SNP sites, determining a fraction of sequence reads that are homozygous for the respective alternative allele (ffAAᵢ) and a fraction of sequence reads that are homozygous for the respective reference allele (ffAAᵢ); and (iii) determining ff according to:

ff=(ffAA+ffBB)/2,
wherein ffAA is a median value of ffAAᵢ across the plurality of informative SNP sites, and ffBB is a median value of ffBBi across the plurality of informative SNP sites.

In some embodiments, α as disclosed herein is determined based at least in part on systemic noise of a sequencing procedure that generates the plurality of sequence reads. In some embodiments, α is determined based at least in part on an empirically measured value of a known paternal allele in fetal-derived nucleic acid molecules at the variant site from a positive test sample. In some embodiments, α is about 1000, 2000, 3000, 4000, or 5000.

In some embodiments, the method of analyzing nucleic acid molecules disclosed herein further comprises prior to the analysis of sequence reads of the nucleic acid molecules, capturing, using a capture probe, the nucleic acid molecules from the biological sample that comprise the target region, and sequencing at least a portion of the captured nucleic acid molecules or amplified products thereof. In some embodiments of the method disclosed herein, at least a portion of the capture probe is complementary to the target region, wherein the SNP site has a reference allele and an alternative allele among individuals in a reference population, wherein the capture probe comprises a sequence selected from a set of four candidate probe sequences, wherein each of the set of four candidate probe sequences is complementary to the target region and comprises a nucleotide selected from A, T, G, and C, respectively, at a position corresponding to the SNP site, and wherein the sequence of the capture probe is a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele.

The methods disclosed herein may be applicable to analysis of either cell-free nucleic acid molecules or cellular nucleic acid molecules, or both. In some embodiments, the biological sample disclosed herein includes whole blood, blood plasma, blood serum, urine, cerebrospinal fluid, buffy coat, vaginal fluid, vaginal flushing fluid, saliva, oral rinse fluid, nasal flushing fluid, a nasal brush sample and a combination thereof. In some embodiments, the biological sample includes blood plasma obtained from a pregnant subject, e.g., a pregnant mother. In some embodiments, the biological sample is obtained from a pregnant mother at first, second, or third trimester. In some embodiments, the biological sample is obtained from a pregnant mother at 1^{st}, 2^{nd}, 3^{rd}, 4^{th}, 5^{th}, 6^{th}, 7^{th}, 8^{th}, 9^{th}, or 10^{th} month into pregnancy.

In some embodiments, the method disclosed herein further includes treating the subject upon detection of presence of chromosomal aneuploidy, chromosomal microdeletion or microduplication, or dominant monogenic variation in the fetus that the subject carries. In some embodiments, the treatment involves pharmaceutical, surgical, occupational, behavioral, or psychological therapies, or any combinations thereof. In some embodiments, the treatment intends to prevent or reduce a risk of the fetus developing a disease or condition. In some embodiments, the treatment intends to ameliorate or eliminate one or more symptoms that the fetus may experience.

In some aspect, provided herein is a computer system, comprising: one or more processors; and a non-transitory computer readable medium comprising instructions operable, when executed by the one or more computer processors, to cause the computer system to perform the method disclosed herein.

In some aspects, provided herein is a non-transitory computer-readable storage medium comprising instructions operable, when executed by one or more processors of a computer system, to cause the computer system to perform the method disclosed herein.

In some aspects, provided herein is a system configured to perform the method disclosed herein.

In some embodiments, the method of the present disclosure uses customized oligonucleotide probes for coordinative allele-aware target enrichment (COATE) to reduce the bias of liquid-phase hybridization kinetics of capture probes toward different allelic loci in the genome, and to improve the capture efficiency and homogeneity of regions of interest for achieving accurate synchronous quantitative analysis of chromosome and gene mutations. In some embodiments, for fetal chromosome copy number detection, next-generation sequencing (NGS) is used to quantitatively analyze maternal and fetal single nucleotide polymorphisms (SNPs) in captured target regions. In some embodiments, statistical methods are used to integrate multiple metrics of cell-free DNA (length of the cell-free DNA, sequencing depth of the target region and allelic mutation rate) with risk factors of different disease (maternal genotype and possible disease inheritance/occurrence patterns) to enable multidimensional analysis of chromosome and genetic variations across parents, chromosomal fragment sizes and cytogenetic mechanisms. There may be currently at least two methods of NIPS using cfDNA sequencing: (1) whole genome low-depth random sequencing (WGS method) and (2) high-depth targeted sequencing (TS method). The WGS method determines the number of targeted fetal chromosome by measuring the ratio of the reads on the targeted chromosome to the reads on the corresponding diploid reference chromosome. Since the WGS method may not be selective for the chromosomal origin of DNA fragments to be sequenced, and chromosomes 21, 18 and 13 represent only 7.85% of the human genome, millions of fragments may need to be sequenced to ensure sufficient counts for chromosomes 21, 18, and 13 to obtain accurate results. In contrast, the high-depth targeted sequencing method may feature dozens of possible fetal normal or abnormal genotypes constructed using maternal genotype information and paternal genotypes estimated from frequencies of SNPs in humans. The theoretical predicted value of minor allele fraction (MAF) for each SNP site is then compared with the actual plasma measurements to calculate the relative probability of each hypothesis. The method may consider only the possible fetal genotypes and does not require the use of diploid reference chromosomes. The present disclosure provides improved approaches that use COATE technology to select a region of a specific target chromosome for the design of target capture probe. Compared with previous NIPS based on multiplex PCR for SNP analysis, methods and systems of the present disclosure may select fewer loci for sequencing analysis and can analyze common human chromosomal aneuploidy and microdeletion diseases more effectively. In addition, methods and systems of the present disclosure may simultaneously select the gene coding regions of common human monogenic dominant genetic diseases, including FGFR3, FGFR2, PTPN11, RAF1, RIT1, SOS1, COL1A1, COL1A2, COL2A1, OTC, MECP2, and other genes as probes to simultaneously detect the process of chromosomal aneuploidy and monogenic mutations, which can effectively detect common dominant monogenic diseases. Monogenic mutation probes can be designed by using capture probe of interest and ordering software tools such as www.idtdna.com/site/order/ngs/%3F.

Chromosomes of interest (chr1-22, chrX, chrY) and SNP sites for the common chromosomal micro-deletion/duplication disorders (affecting CNV regions of 0.5 Mb or larger in size) may be selected for probe design. Any fetal variation, either single nucleotide or chromosomal variation can be detected in maternal plasma as long as the fetal and maternal genotypes are not exactly the same. In NGS, this detectability depends on the fetal cell-free DNA fraction (fetal DNA as a percentage of total maternal plasma cfDNA) and the sequencing depth. Although whole genome low-depth sequencing can be used to detect certain nondiploids, this method may not be applicable to smaller chromosomal copy number variants or genetic variants at the gene level. To detect all fetal genetic variants in maternal plasma, targeted enrichment methods can be used, including probe hybridization or PCR amplification of regions of interest for directed high-depth sequencing. Because liquid-phase hybridization using DNA oligonucleotides may not require region-specific primers, this has the advantage of fewer allele drop-outs in enriching highly fragmented cfDNA. However, probe oligonucleotides have different hybridization thermodynamics for different individual target regions, as even a single non-complementary base between the target region and the probe may result in different hybridization thermodynamics. This raises the issue that NGS-based detection methods rely on accurate genotyping and quantification of the diallele fraction for the detection of copy number variation in NIPS. In NGS, the central allele fraction (CAF) of a germ-line heterozygous variant may be expected to be 50% when the sampling (DNA input) and sequencing (sequencing depth) are sufficient. However, using current NGS techniques, the CAF measured in euploid samples is not always exactly 50%, due to unavoidable experimental errors introduced by different site-specific hybridization kinetics [14]. If the error in measured euploid CAF is too large, it can mask A F changes caused by gene copy number variation in the fetus in maternal plasma. When conventional probe design is based on reference sequences, we found CAFs consistently below 50.0% across >2,000 allelic loci, with a range of 43.1-49.4%. Such a systematic bias may indicate that the hybridization efficiency between the probe and the region of interest with the mutation (minor allele) is slightly lower because the probe is usually designed based on the reference allele (usually major allele). In some embodiments, a coordinated design (COATE) of probes is performed for the chromosome aneuploidy, microdeletion/microduplication, and monogenic target region allele of interest, which suppressed the bias of allele hybridization kinetics.

The COATE method used herein can allow calculation of the difference in hybridization annealing temperature (ΔTm) between the probe and the target including the reference and mutant alleles. In any given single nucleotide diallele locus, there are four probes (-A-, -G-, -C-, -T-), two of which are complementary to the reference or mutant allele and the other two are not complementary to the reference or mutant allele. Unlike conventional probe designs, such probe combinations may not require complementarity with reference genomic sequences or mutant sequences; these probes may or may not be complementary to the reference or mutant alleles, and it is only necessary that the probes have minimal ΔTm to the reference gene sequence (wild-type) and mutant sequence (mutant-type) in the capture region.

An allele of the SNP sites with relatively high distribution in populations is called wild-type (B), and an allele with relatively low distribution in populations is called mutant-type (A), the homozygous wild genotype is BB, the homozygous mutant genotype is AA, and the heterozygous genotype is AB. In some embodiments, the sequence selection of these probes follows the following principle: for every 100 nucleotides in the reference genomic sequence, the probe sequence contains up to 10 nucleotides different from the reference genomic sequence, and the rest are identical to the reference genomic sequence. In order to obtain polynucleotides whose nucleotide sequences are at least 90% identical to the reference genomic sequence, up to 10% of the nucleotides in the reference genomic sequence may be substituted by other nucleotides or deleted; or some nucleotides may be inserted into the reference sequence, wherein the inserted nucleotides may be up to 10% of the total nucleotides of the reference sequence; or in some probes, there is a combination of deletions, insertions and substitutions, wherein the deleted, inserted and substituted nucleotides are up to 10% of the total nucleotides of the reference sequence. These deletions, insertions and substitutions in the reference sequence may occur at the 5′ or 3′ end of the reference nucleotide sequence, or anywhere therebetween, and they are either scattered in the of the reference sequence alone or present in one or more adjacent groups in the reference sequence.

The detection method provided herein may be innovative for at least the following reasons. There may be two analytical methods for NIPS by sequencing maternal plasma cell-free DNA: the low-depth whole genome sequencing (WGS) method and the single nucleotide polymorphism (SNP) method with high-depth targeted sequencing. The WGS method may determine the ploidy of targeted fetal chromosome by measuring the ratio of the reads on the targeted chromosome to the reads of the corresponding diploid reference chromosome. Since the WGS method may not be selective for the chromosomal origin of DNA fragments to be sequenced, and chromosomes 21, 18, and 13 represent only 7.85% of the human genome, millions of fragments may need to be sequenced to ensure sufficient counts for chromosomes 21, 18, and 13 to obtain high confidence results. The SNP method, on the other hand, may be directed to analyze only some of locus in the landmark regions of the chromosomes of interest and therefore, the amount of DNA need for sequencing can be significantly reduced compared to the WGS method. The method is based on maternal genotype information and the paternal genotype calculated from the frequencies of SNPs in humans, which is used to construct possible fetal normal or abnormal genotypes. The theoretical predicted value of minor allele fraction (MAF) for each SNP site is then compared with the actual plasma measurements and the relative likelihood of each hypothesis is calculated. This method may consider only the possible fetal genotypes and does not require the use of diploid reference chromosomes as in the WGS method, thereby reducing the requirements for experimental manipulation and data analysis. The current SNP method may be based on multiplex PCR technique, and this amplification technique may be prone to allele drop-out (ADO) in the analysis of highly fragmented cell-free DNA, thus tens of thousands at SNP sites need to be analyzed simultaneously to improve the signal-to-noise ratio for chromosome copy number quantification. To address this problem, the present method uses an innovative liquid-phase hybridization technique to selectively capture polymorphic loci for sequencing, avoiding the use of region-specific amplification primers and reducing the probability of ADO occurrence. Moreover, the present technique for SNPs in the region of interest may be designed by using customized oligonucleotide probes (coordinative allele-aware target enrichment), which can reduce the bias of liquid-phase hybridization kinetics of capture probes toward different allelic loci in the genome, improve the capture efficiency and homogeneity of the region of interest, and achieve accurate quantitative chromosome analysis. Based on the above innovative technologies, the present method can achieve highly efficient detection of common chromosomal aneuploidy and microdeletion/microduplication diseases by sequencing and analyzing only 2320 SNP sites, and has a significantly reduced number of loci compared to the previous multiplex PCR-based SNP analysis method.

In some embodiments, disclosed herein is a product for non-invasive detection based on the hybridization capture method, which is used for synchronous detection of chromosomal aneuploidy, microdeletion/microduplication and dominant monogenic diseases, and is more comprehensive than the traditional NIPS in the types of diseases of synchronous detection.

In some embodiments, disclosed herein is a product for non-invasive detection using SNP-based hybridization capture method, which is less affected by interfering factors than the WGS detection method, such as not being affected by the ratio of GC content, not being affected by the genotype of the fetal mother to be examined, and not being interfered by other samples within the same batch.

In some embodiments, disclosed herein is a product for non-invasive detection using SNP-based hybridization capture method, which requires fewer SNP sites compared to SNP-based multiplex PCR method.

In some embodiments, disclosed herein a detection method for non-invasive prenatal screening of fetuses. In some embodiments, disclosed herein is a detection method for chromosome copy number variation, chromosome microdeletion/microduplication, and/or dominant monogenic variation. In some embodiments, disclosed herein is a method of designing a targeted capture probe for non-invasive prenatal screening of fetuses. In some embodiments, disclosed herein is a detection kit for non-invasive prenatal screening of fetuses. In some embodiments, disclosed herein is a device for non-invasive prenatal screening of fetuses. In some embodiments, disclosed herein is a computer-readable storage medium for non-invasive prenatal screening of fetuses. In some embodiments, disclosed herein is a system for non-invasive prenatal screening of fetuses. In some embodiments, disclosed herein is a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses.

In some embodiments, disclosed herein a detection method for non-invasive prenatal screening of fetuses. In some embodiments, disclosed herein is use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the detection method for non-invasive prenatal screening of fetuses comprises the following operations:
(1) detecting and calculating fetal fraction (ff) of cell-free nucleic acids;
(2) selecting one or more SNP sites in a chromosome to be detected, wherein an allele of the SNP sites with relatively high distribution in populations is called wild-type (B), and an allele with relatively low distribution in populations is called mutant-type (A), the homozygous wild genotype is BB, the homozygous mutant genotype is AA, and the heterozygous genotype is AB;
   in some embodiments, the allele with relatively high distribution in populations is: allele B identical to the reference genome sequence in the human genome assembly build hg38; and the allele with relatively low distribution in populations is: allele A different from the reference genome sequence in the human genome assembly build hg38;
(3) using a targeted capture probe for the one or more SNP sites to capture cell-free DNA (cfDNA) in maternal peripheral blood, and sequencing the cfDNA after amplification to obtain the reads NA of the allele A and the sequencing depth N at the site(s);
   in some embodiments, allele A is a mutant-type gene, and the reads NA of allele A refers to the reads of mutant-type allele A; allele B is a wild-type gene, and the reads NB of allele B refers to the reads of wild-type allele B; the sequencing depth N at the site is the sum of the reads NA of allele A and the reads NB of allele B;
   in some embodiments, the fetal cell-free nucleic acid is obtained through the detection of cell-free nucleic acids in maternal peripheral blood, wherein the detection of the cell-free nucleic acids in maternal peripheral blood comprises the detections of the mother's own cell-free nucleic acid and the cell-free nucleic acid of the fetus;
(4) calculating the probability that a fetus may have a normal chromosome copy number or abnormal different copy numbers at each SNP site; and calculating the probability values of the fetus being euploid or aneuploid, respectively, based on the percentage of mutant genotype in the cfDNA (A %) actually measured for each SNP site, the fetal fraction (ff) of cell-free nucleic acids and the mother's genotype at the site;
   wherein the maximum value among the sums of the probabilities at all valid SNP sites in the same chromosome is the interpreted karyotype of the fetus;
in some embodiments, the valid SNP sites are all the SNP sites where the genotypes of the fetus and those of the mother are not completely the same;
   the calculated fetal karyotype H includes: D (disomy), MI (maternal trisomy type I), MII (maternal trisomy type II), PI (paternal trisomy type I), PII (paternal trisomy type II), LM (maternal microdeletion) and LP (paternal microdeletion);
   the karyotype probabilities of the fetus at each SNP site is obtained by taking logarithm of the linear combination of π-weighted conditional beta binomial distribution probabilities, and the calculation equation is as follows:

$$log ( p ( NAi , N , pAi , H ) ) = log ( {\sum }_{k} π k Beta - Binom ( pAi , N , α , β ) )$$

i is the i-th valid SNP site;
N is the sequencing depth at the SNP site; pAi is the expected value of the reads percentage of a mutant-type from the next generation sequencing (NGS) at different gene loci of euploid or aneuploid fetus; when the fetus has different karyotypes, pAi is of different genotypes at different loci H, and their expected values will vary from each other; pAi of specific different loci H is shown in Table 1;

TABLE 1
calculation of expected center frequency of mutant genotype of fetus with different karyotypes

| MAT | TYPE | AA | AB | BB | AAA | AAB | ABB | BBS | A | B |
|---|---|---|---|---|---|---|---|---|---|---|
| AA | D | 1 | 1 − ff/2 | 0 | 0 | 0 | 0 | 0 | 0 | 0 |
| AB | D | 0.5 − ff/2 | 0.5 | 0.5 + ff/2 | 0 | 0 | 0 | 0 | 0 | 0 |
| BB | D | 0 | ff/2 | 0 | 0 | 0 | 0 | 0 | 0 | 0 |
| AA | MI | 0 | 0 | 0 | 1 | 1 − ffc/3 | 0 | 0 | 0 | 0 |
| AB | MI | 0 | 0 | 0 | 0 | 0.5 + ffc/6 | 0.5 − ffc/6 | 0 | 0 | 0 |
| BB | MI | 0 | 0 | 0 | 0 | 0 | ffc/3 | 0 | 0 | 0 |
| AA | MII | 0 | 0 | 0 | 1 | 1 − ffc/3 | 0 | 0 | 0 | 0 |
| AB | MII | 0 | 0 | 0 | 0.5 + ffc/2 | 0.5 + ffc/5 | 0.5 − ffc/6 | 0.5 − ffc/2 | 0 | 0 |
| BB | MII | 0 | 0 | 0 | 0 | 0 | ffc/3 | 0 | 0 | 0 |
| AA | PI | 0 | 0 | 0 | 1 | 1 − ffc/3 | 1 − 2ffc/3 | 0 | 0 | 0 |
| AB | PI | 0 | 0 | 0 | 0.5 + ffc/2 | 0.5 + ffc/6 | 0.5 − ffc/6 | 0.5 − ffc/2 | 0 | 0 |
| BB | PI | 0 | 0 | 0 | 0 | 2ffc/3 | ffc/3 | 0 | 0 | 0 |
| AA | PII | 0 | 0 | 0 | 0 | 0 | 1 − 2ffc/3 | 0 | 0 | 0 |
| AB | PII | 0 | 0 | 0 | 0.5 + ffc/2 | 0.5 + ffc/6 | 0.5 − ffc/6 | 0.5 − ffc/2 | 0 | 0 |
| BB | PII | 0 | 0 | 0 | 0 | 2ffc/3 | 0 | 0 | 0 | 0 |
| AA | LM | 0 | 0 | 0 | 0 | 0 | 0 | 0 | 1 | 1 − ffc |
| AB | LM | 0 | 0 | 0 | 0 | 0 | 0 | 0 | 0.5 + ffc/2 | 0.5 − ffc/2 |
| BB | LM | 0 | 0 | 0 | 0 | 0 | 0 | 0 | ffc | 0 |
| AA | LP | 0 | 0 | 0 | 0 | 0 | 0 | 0 | 1 | 0 |
| AB | LP | 0 | 0 | 0 | 0 | 0 | 0 | 0 | 0.5 + ffc/2 | 0.5 − ffc/2 |
| BB | LP | 0 | 0 | 0 | 0 | 0 | 0 | 0 | 0 | 0 |

ffc is the corrected fetal fraction when the fetus is aneuploid;
when the fetus has trisomy, ffc=1.5ff/(1+0.5ff)=3ff/(2+ff); when the fetus has chromosome deletion, ffc=0.5ff/(1−0.5ff)=ff/(2−ff)
α is a discrete parameter selected for pAi based on the actual value in sequencing; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β=α/pAi−α
calculation of the weighting coefficient πk is based on different karyotypes of the fetus:

$$π k = {\sum }_{PATK} p ( FET ) \times p ( PATk )$$

wherein PATkϵ{AA, AB, BB}, p(PATk) is calculated according to the Hardy-Weinberg equation, and the allele frequencies at the SNP site are p:

p(AA)=p×p

p(AB)=2×p×(1−p)

p(BB)=(1−p)×(1−p)
in some embodiments, the allele frequency p at the SNP site comes from a public database, in some embodiments is selected from the 1000 Genomes database;
p(FET) is the possible genotype of the fetus, which is affected by the genotypes of father and mother, when the fetus is euploid or aneuploid, p(FET) is calculated according to Mendel's Laws of Inheritance, as shown in Table 2;

TABLE 2
calculation of probability of fetal genotype

| MAT | BAT | TYPE | AA | AB | BB | AAA | AAB | ABB | BBB | A | B |
|---|---|---|---|---|---|---|---|---|---|---|---|
| AA | AA | D | 1 | 0 | 0 | 0 | 0 | 0 | 0 | 0 | 0 |
| AA | AB | D | 0.5 | 0.5 | 0 | 0 | 0 | 0 | 0 | 0 | 0 |
| AA | BB | D | 0 | 1 | 0 | 0 | 0 | 0 | 0 | 0 | 0 |
| AB | AA | D | 0.5 | 0.5 | 0 | 0 | 0 | 0 | 0 | 0 | 0 |
| AB | AB | D | 0.25 | 0.5 | 0.25 | 0 | 0 | 0 | 0 | 0 | 0 |
| AB | BB | D | 0 | 0.5 | 0.5 | 0 | 0 | 0 | 0 | 0 | 0 |
| BB | AA | D | 0 | 1 | 0 | 0 | 0 | 0 | 0 | 0 | 0 |
| BB | AB | D | 0 | 0.5 | 0.5 | 0 | 0 | 0 | 0 | 0 | 0 |
| BB | BB | D | 0 | 0 | 1 | 0 | 0 | 0 | 0 | 0 | 0 |
| AA | AA | MI | 0 | 0 | 0 | 1 | 0 | 0 | 0 | 0 | 0 |
| AA | AB | MI | 0 | 0 | 0 | 0.5 | 0.5 | 0 | 0 | 0 | 0 |
| AA | BB | MI | 0 | 0 | 0 | 0 | 1 | 0 | 0 | 0 | 0 |
| AB | AA | MI | 0 | 0 | 0 | 0 | 1 | 0 | 0 | 0 | 0 |
| AB | AB | MI | 0 | 0 | 0 | 0 | 0.5 | 0.5 | 0 | 0 | 0 |
| AB | BB | MI | 0 | 0 | 0 | 0 | 0 | 1 | 0 | 0 | 0 |
| BB | AA | MI | 0 | 0 | 0 | 0 | 0 | 1 | 0 | 0 | 0 |
| BB | AB | MI | 0 | 0 | 0 | 0 | 0 | 0.5 | 0.5 | 0 | 0 |
| BB | BB | MI | 0 | 0 | 0 | 0 | 0 | 0 | 1 | 0 | 0 |
| AA | AA | MII | 0 | 0 | 0 | 1 | 0 | 0 | 0 | 0 | 0 |
| AA | AB | MII | 0 | 0 | 0 | 0.5 | 0.5 | 0 | 0 | 0 | 0 |
| AA | BB | MII | 0 | 0 | 0 | 0 | 1 | 0 | 0 | 0 | 0 |
| AB | AA | MII | 0 | 0 | 0 | 0.5 | 0 | 0.5 | 0 | 0 | 0 |
| AB | AB | MII | 0 | 0 | 0 | 0.25 | 0.25 | 0.25 | 0.25 | 0 | 0 |
| AB | BB | MII | 0 | 0 | 0 | 0 | 0.5 | 0 | 0.5 | 0 | 0 |
| BB | AA | MII | 0 | 0 | 0 | 0 | 0 | 1 | 0 | 0 | 0 |
| BB | AB | MII | 0 | 0 | 0 | 0 | 0 | 0.5 | 0.5 | 0 | 0 |
| BB | BB | MII | 0 | 0 | 0 | 0 | 0 | 0 | 1 | 0 | 0 |
| AA | AA | PI | 0 | 0 | 0 | 1 | 0 | 0 | 0 | 0 | 0 |
| AA | AB | PI | 0 | 0 | 0 | 0 | 1 | 0 | 0.5 | 0 | 0 |
| AA | BB | PI | 0 | 0 | 0 | 0 | 0 | 1 | 0 | 0 | 0 |
| AB | AA | PI | 0 | 0 | 0 | 0.5 | 0.5 | 0 | 0 | 0 | 0 |
| AB | AB | PI | 0 | 0 | 0 | 0 | 0.5 | 0.5 | 0 | 0 | 0 |
| AB | BB | PI | 0 | 0 | 0 | 0 | 0 | 0.5 | 0.5 | 0 | 0 |
| BB | AA | PI | 0 | 0 | 0 | 0 | 1 | 0 | 0 | 0 | 0 |
| BB | AB | PI | 0 | 0 | 0 | 0 | 0 | 1 | 0 | 0 | 0 |
| BB | BB | PI | 0 | 0 | 0 | 0 | 0 | 0 | 1 | 0 | 0 |
| AA | AA | PII | 0 | 0 | 0 | 1 | 0 | 0 | 0 | 0 | 0 |
| AA | AB | PII | 0 | 0 | 0 | 0.5 | 0 | 0.5 | 0 | 0 | 0 |
| AA | BB | PII | 0 | 0 | 0 | 0 | 0 | 1 | 0 | 0 | 0 |
| AB | AA | PII | 0 | 0 | 0 | 0.5 | 0.5 | 0 | 0 | 0 | 0 |
| AB | AB | PII | 0 | 0 | 0 | 0.25 | 0.25 | 0.25 | 0.25 | 0 | 0 |
| AB | BB | PII | 0 | 0 | 0 | 0 | 0 | 0.5 | 0.5 | 0 | 0 |
| BB | AA | PII | 0 | 0 | 0 | 0 | 1 | 0 | 0 | 0 | 0 |
| BB | AB | PII | 0 | 0 | 0 | 0 | 0.5 | 0 | 0.5 | 0 | 0 |
| BB | BB | PII | 0 | 0 | 0 | 0 | 0 | 0 | 1 | 0 | 0 |
| AA | AA | LM | 0 | 0 | 0 | 0 | 0 | 0 | 0 | 1 | 0 |
| AA | AB | LM | 0 | 0 | 0 | 0 | 0 | 0 | 0 | 0.5 | 0.5 |
| AA | BB | LM | 0 | 0 | 0 | 0 | 0 | 0 | 0 | 0 | 1 |
| AB | AA | LM | 0 | 0 | 0 | 0 | 0 | 0 | 0 | 1 | 0 |
| AB | AB | LM | 0 | 0 | 0 | 0 | 0 | 0 | 0 | 0.5 | 0.5 |
| AB | BB | LM | 0 | 0 | 0 | 0 | 0 | 0 | 0 | 0 | 1 |
| BB | AA | LM | 0 | 0 | 0 | 0 | 0 | 0 | 0 | 1 | 0 |
| BB | AB | LM | 0 | 0 | 0 | 0 | 0 | 0 | 0 | 0.5 | 0.5 |
| BB | BB | LM | 0 | 0 | 0 | 0 | 0 | 0 | 0 | 0 | 1 |
| AA | AA | LP | 0 | 0 | 0 | 0 | 0 | 0 | 0 | 1 | 0 |
| AA | AB | LP | 0 | 0 | 0 | 0 | 0 | 0 | 0 | 1 | 0 |
| AA | BB | LP | 0 | 0 | 0 | 0 | 0 | 0 | 0 | 1 | 0 |
| AB | AA | LP | 0 | 0 | 0 | 0 | 0 | 0 | 0 | 0.5 | 0.5 |
| AB | AB | LP | 0 | 0 | 0 | 0 | 0 | 0 | 0 | 0.5 | 0.5 |
| AB | BB | LP | 0 | 0 | 0 | 0 | 0 | 0 | 0 | 0.5 | 0.5 |
| BB | AA | LP | 0 | 0 | 0 | 0 | 0 | 0 | 0 | 0 | 1 |
| BB | AB | LP | 0 | 0 | 0 | 0 | 0 | 0 | 0 | 0 | 1 |
| BB | BB | LP | 0 | 0 | 0 | 0 | 0 | 0 | 0 | 0 | 1 |

calculation of maternal genotype: if NA/N≤0.2, maternal genotype is BB; if 0.3<NA/N<0.8, maternal genotype is AB; and if NA/N≥0.8, maternal genotype is AA.

(5) calculation of fetal chromosome copy number variation,
during sperm or egg production, if a certain chromosome under examination does not undergo meiotic homologous recombination, the calculation equation for the distribution difference between probabilities of an abnormal chromosome copy number and a normal chromosome copy number is as follows:

$$Δ L = {\sum }_{1}^{M} ( log ( L D i ) - log ( L H i ) )$$

Hϵ{MI, MII, PI, PII, LM, LP}
LD is the probability value at the site in the euploid karyotype;
LH is the probability value at the site in the aneuploid karyotype;
M is the number of valid SNP sites in the chromosome;
chromosomal aneuploidy is positive when ΔL is less than a detection threshold; the detection threshold is determined by the detection results of pregnant women's plasma samples with known prenatal diagnosis results and artificial mixtures of positive and negative reference samples; and the detection thresholds for negative samples and positive samples, specific to the different aneuploid types, are shown in Table 3;

TABLE 3
detection thresholds for negative samples and positive samples

| Karyotype | Positive | Negative | Grey Area |
|---|---|---|---|
| MI | <−10 | >−4 | [−10, −4] |
| MII | <−5 | >+5 | [−5, +5] |
| PI | <−20 | >−10 | [−20, −10] |
| PII | <−20 | >−10 | [−20, −10] |
| LM | <−10 | >−5 | [−10, −5] |
| LP | <−10 | >−5 | [−10, −5] |

In some embodiments, the method is a detection method for chromosome copy number.

In one embodiment, provided herein is a detection method for non-invasive prenatal screening of fetuses, wherein the operation (5) of the detection method for non-invasive prenatal screening of fetuses is:

(5) calculation of fetal chromosome microdeletion/microduplication,
during sperm or egg production, if a certain chromosome under examination is partially deleted or partially duplicated, the calculation equation for the distribution difference between probabilities of an abnormal chromosome copy number and a normal chromosome copy number is as follows:

$$Δ L = min ( {\sum }_{b 1}^{b 2} ( log ( L D i ) - log ( L H i ) ) )$$

Hϵ{MI, MIT, PI, PII, LM, LP}, 0<b1, b2<M
b1 and b2 are the starting and ending positions at which the chromosome undergoes microdeletion/microduplication, respectively;
chromosomal aneuploidy is positive when ΔL is less than a detection threshold; the detection threshold is determined by the detection results of pregnant women's plasma samples with known prenatal diagnosis results and artificial mixtures of positive and negative reference samples; the detection thresholds for negative samples and positive samples are shown in Table 3; and the method is a detection method for chromosome microdeletion/microduplication;

or (5) calculation of dominant monogenic variation,
dominant monogenic variation occur in regions where the mother is homozygous wild-type BB; the probability that the A reads are from the fetus is calculated based on the reads NA of A, the sequencing depth N at the site, and the fetal fraction ff of cell-free nucleic acids through a beta binomial distribution fitting, and the calculated probability is compared with the probability of systematic noise, wherein:
at a certain locus, the probability that the fetus has paternal or de novo mutations when the mother is homozygous wild-type BB is:

ΔL=log(beta−binom(pAi,N,α,β1))−log(beta−binom(e,N,α,β2))
in some embodiments, pAi=ff/2,

$$Δ L = log ( beta - binom ( \frac{ff}{2} , N , α , β 1 ) - log ( beta - binom ( e , N , α , β 2 ) )$$

N is the sequencing depth at the site;
ff is the fetal fraction of cell-free nucleic acids;
α is a discrete parameter selected based on the actually measured value of the paternal allele in the fetal cell-free DNA; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β1=2×α/ff−α;
e is the systematic error rate at the site, and the systematic error rate is the ratio of mutant genotypes at the site in known negative samples; a is an actually measured discrete parameter of systematic noise, and the range of α is determined to be 1000-5000; in some embodiments, the value of a is 1000, 2000, 3000, 4000, or 5000;

β2=α/e−α
when ΔL is greater than the detection threshold which is 1, the gene mutation is positive; and the method is a detection method for dominant monogenic variation.

The log used in methods and systems of the present disclosure represents the value of log base e, wherein log(x) represents the natural logarithm, and its base value is e.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the detection method for non-invasive prenatal screening of fetuses further comprises: one or more combinations of calculation of fetal chromosome copy number variation, calculation of fetal chromosome microdeletion/microduplication, and calculation of dominant monogenic variation;

wherein the calculation of fetal chromosome microdeletion/microduplication is as follows:
during sperm or egg production, if a certain chromosome under examination is partially deleted or partially duplicated, the calculation equation for the distribution difference between probabilities of an abnormal chromosome copy number and a normal chromosome copy number is as follows:

$$Δ L = min ( {\sum }_{b 1}^{b 2} ( log ( L D i ) - log ( L H i ) ) )$$

Hϵ{MI, MII, PI, PII, LM, LP}, 0<b1, b2<M
b1 and b2 are the starting and ending positions at which the chromosome undergoes microdeletion/microduplication, respectively;
chromosomal aneuploidy is positive when ΔL is less than a detection threshold; the detection threshold is determined by the detection results of pregnant women's plasma samples with known prenatal diagnosis results and artificial mixtures of positive and negative reference samples; and the detection thresholds for negative samples and positive samples are shown in Table 3;
the calculation of dominant monogenic variation is as follows:
dominant monogenic variation occur in regions where the mother is homozygous wild-type BB; the probability that the A reads are from the fetus is calculated based on the reads NA of A, the sequencing depth N at the site, and the fetal fraction ff of cell-free nucleic acids through a beta binomial distribution fitting, and the calculated probability is compared with the probability of systematic noise, wherein: at a certain locus, the probability that the fetus has paternal or de novo mutations when the mother is homozygous wild-type BB is:

$$Δ L = log ( beta - binom ( \frac{ff}{2} , N , α , β 1 ) ) - log ( beta - binom ( e , N , α , β 2 ) )$$

N is the sequencing depth at the site;
ff is the fetal fraction of cell-free nucleic acids;
α is a discrete parameter selected based on the actually measured value of the paternal allele in the fetal cell-free DNA; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β1=2×α/ff−α;
e is the systematic error rate at the site, and the systematic error rate is the ratio of mutant genotypes at the site in known negative samples; a is an actually measured discrete parameter of systematic noise, and the range of α is determined to be 1000-5000; in some embodiments, the value of a is 1000, 2000, 3000, 4000, or 5000;

β2=α/e−α
when ΔL is greater than the detection threshold which is 1, the gene mutation is positive; and the method is a detection method for fetal chromosome copy number variation, fetal chromosome microdeletion/microduplication, and/or dominant monogenic variation.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the detection method for non-invasive prenatal screening of fetuses comprises: calculation of fetal chromosome copy number variation; or calculation of fetal chromosome microdeletion/microduplication; or calculation of dominant monogenic variation; or calculation of fetal chromosome copy number variation and calculation of fetal chromosome microdeletion/microduplication; or calculation of fetal chromosome copy number variation and calculation of dominant monogenic variation; or calculation of fetal chromosome microdeletion/microduplication and calculation of dominant monogenic variation; or calculation of fetal chromosome copy number variation, calculation of fetal chromosome microdeletion/microduplication and calculation of dominant monogenic variation, wherein the method is a detection method for fetal chromosome copy number variation, fetal chromosome microdeletion/microduplication and/or dominant monogenic variation.

In some embodiments, the detected gene mutation is only an intermediate result, and it cannot directly determine whether the fetus has a specific disease. For gene mutations that meet the detection threshold, further clinical data interpretation is required. Therefore, the detection method of the present disclosure may not be used for disease diagnosis.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein methods and systems of the present disclosure have no limitation on the method for calculating the fetal fraction (ff) of cell-free nucleic acids, and the detection and calculation can be carried out by any method well-known to those of ordinary skill in the art.

In some embodiments, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the operation (1) detects and calculates the fetal fraction (ff) of cell-free nucleic acids,

and comprises the following operations:
when the mother is homozygous wild-type BB, the genotype of the fetus may be BB or BA, thus for the sites where the fetus is BA, the ratio distribution of reads A is centered on ff/2, and the fetal fraction of cell-free nucleic acids can be calculated by the median value ffBB of the ratio of reads A for all sites of this type; when the mother is homozygous mutant-type AA, the genotype of the fetus may be AA or AB, thus for the sites where the fetus is AB, the ratio distribution of reads A is centered on ff/2, and the fetal fraction of cell-free nucleic acids can be calculated by the median value ffAA of the ratio of reads B for all sites of this type; the fetal fraction (ff) of cell-free nucleic acids is calculated as follows:

ff=(ffAA+ffBB)/2
in some embodiments, when detecting and calculating the fetal fraction of cell-free nucleic acids, any chromosome site can be selected;
more in some embodiments, sites in the human genome where the copy number rarely changes are selected; further in some embodiments, sites in the human genome where the copy number rarely changes are selected; and these sites include or does not include sites in chromosomes 13, 18, 21, 22, X, and Y.

In some embodiments, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the SNP site to be detected is one or more SNP sites selected from the chromosome to be detected, and is one or more of all chromosomes containing SNP sites; in some embodiments, the SNP site to be detected is one or more of chromosomes 13, 18, 21, 22, X, and Y.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the equations for the sum of the probabilities at the chromosomal SNP sites in the case where one chromosomal recombination may occur during the production of parental germ cells are:

$$Δ L = min ( {\sum }_{1}^{k} ( log ( L D i ) - log ( L H 1 i ) ) + {\sum }_{k + 1}^{M} ( log ( L D i ) - log ( L H 2 i ) ) )$$

$$Δ L = min ( {\sum }_{1}^{k} ( log ( L D i ) - log ( L H 2 i ) ) + {\sum }_{k + 1}^{M} ( log ( L D i ) - log ( L H 1 i ) ) )$$

H1, H2ϵ{MI, MII, PI, PII}; chromosomal aneuploidy is positive when one of the above two calculation results is less than the detection threshold in Table 3; and the detection thresholds for negative samples and positive samples are shown in Table 3.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the equations for the sum of the probabilities at the chromosomal SNP sites in the case where one or two chromosomal recombinations may occur during the production of parental germ cells are:

$$Δ L ( H 121 ) = min ( {\sum }_{1}^{b 1} ( log ( LDi ) - log ( LH 1 i ) ) + {\sum }_{b 1}^{b 2} ( log ( LDi ) - log ( LH 2 i ) ) + {\sum }_{b 2}^{M} ( log ( LDi ) - log ( LH 1 i ) )$$

$$Δ L ( H 212 ) = min ( {\sum }_{1}^{b 1} ( log ( LDi ) - log ( LH 2 i ) ) + {\sum }_{b 1}^{b 2} ( log ( LDi ) - log ( LH 1 i ) ) + {\sum }_{b 2}^{M} ( log ( LDi ) - log ( LH 2 i ) ) )$$

H1, H2ϵ{MI, MII, PI, PII},
b1 and b2 are the calculated positions where the chromosome recombinations occur; chromosomal aneuploidy is positive when one of the above two calculation results is less than the detection threshold; and the detection thresholds for negative samples and positive samples are shown in Table 3.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the targeted capture probe covers all genes containing gene mutations; in some embodiments, the targeted capture probe covers the following genes: FGFR3, FGFR2, PTPN11, RAF1, RIT1, SOS1, COL1A1, COL1A2, COL2A1, OTC and MECP2.

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the selection of one or more SNP sites in the chromosome to be detected is to prioritize sites with a simple structure and a GC content close to 40-60% based on the human genome sequence assembly build hg38.

In some embodiments, based on 1000G and gnomAD public databases, the sites having an allele frequency close to 0.3 to 0.7 are selected, and these sites include a total of at least 2320 SNP sites in chromosomes 1 to 22, X and Y.

The URLs of the public databases used are as below:
Human genome hg38:
hgdownload.cse.ucsc.edu/goldenpath/hg38/chromosomes/1000G:
www.internationalgenome.org/data/gnomAD:
gnomad.broadinstitute.org/

In one embodiment, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the targeted capture probe used in the operation (3) is obtained using the following method of designing a targeted capture probe and the method comprises the following operations:
(1) determining the SNP site of interest;
(2) for each SNP site of targeted capture, designing four probes based on the SNP site, wherein the four probes are designed as -A-, -G-, -C-, -T- at the SNP site, respectively; and
(3) for each SNP site of targeted capture, calculating the annealing temperatures (Tm) for the binding of the four probes to two target sequences, respectively, wherein the two target sequences each carry two different single nucleotide polymorphisms; calculating the difference in annealing temperatures (ΔTm) for the binding of the four probes to the two target sequences based on the annealing temperature (Tm); and based on the calculation results, selecting the probe with the lowest ΔTm among the four probes and determining it as the optimal probe for the site.

In some embodiments, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein in the method of designing a targeted capture probe, the two target sequences are used as a reference gene sequence of the wild-type and a mutant gene sequence of the mutant-type, respectively; wherein the Tm values for the binding of the four probes to the reference gene sequence of the wild-type are: Tma, Tmg, Tmc, and Tmt, respectively, the Tm values for the binding of the four probes to the mutant gene sequence of the mutant-type are: Tma′, Tmg′, Tmc′, and Tmt′, respectively, and the ΔTm values for the binding of the four probes to the two target sequences are: |Tma−Tma′|, |Tmg−Tmg′|, |Tmc−Tmc′|, and |Tmt−Tmf|, respectively.

In some embodiments, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein in the method of designing a targeted capture probe, the annealing temperature (Tm) for the probes is calculated using a nearest neighbor model and cation correction, and the calculation equation for the annealing temperature (Tm) for the probes is as below:

$${T}_{m} = \frac{Δ H}{Δ S + R \times ln {C}_{T}} + 16.6 log [ {Na}^{+} ]$$

ΔH represents the sum of standard enthalpy changes for all adjacent base pairs, ΔS represents the sum of standard entropy changes for all adjacent base pairs, R is the molar gas constant, CT represents the concentration of the primers, and [Na+] represents the concentration of monovalent sodium ions in solution.

In some embodiments, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein in the method of designing a targeted capture probe, the operation (2) is for each SNP site of targeted capture, designing four probes based on the SNP site, wherein the four probes are designed as -A-, -G-, -C-, -T- at the SNP site, respectively, and the rest positions are complementary to the sequence of interest.

In some embodiments, provided herein is a detection method for non-invasive prenatal screening of fetuses, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the probe has a length of 100-200 bp; in some embodiments, the probe has a length of 100-190 bp or 100-180 bp or 100-170 bp or 100-160 bp or 100-150 bp or 100-140 bp or 100-130 bp or 100-120 bp or 110-200 bp or 110-190 bp or 110-180 bp or 110-170 bp or 110-160 bp or 110-150 bp or 110-140 bp or 110-130 bp or 110-120 bp; further, the probe has a length of 100 bp, 110 bp, 120 bp, 130 bp, 140 bp, 150 bp, 160 bp, 170 bp, 180 bp, 190 bp or 200 bp.

In some embodiments, disclosed herein is a detection method for chromosome copy number variation, chromosome microdeletion/microduplication, and/or dominant monogenic variation, which is for non-diagnostic purposes. In some embodiments, disclosed herein is use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, comprising the following operations:
(1) detecting and calculating fetal fraction (ff) of cell-free nucleic acids;
(2) selecting one or more SNP sites in a chromosome to be detected, wherein an allele of the SNP sites with relatively high distribution in populations is called wild-type (B), and an allele with relatively low distribution in populations is called mutant-type (A), the homozygous wild genotype is BB, the homozygous mutant genotype is AA, and the heterozygous genotype is AB;

in some embodiments, the allele with relatively high distribution in populations is: allele B identical to the reference genome sequence in the human genome assembly build hg38; and the allele with relatively low distribution in populations is: allele A different from the reference genome sequence in the human genome assembly build hg38;
(3) using a targeted capture probe for the one or more SNP sites to capture cell-free DNA (cfDNA) in maternal peripheral blood, and sequencing the cfDNA after amplification to obtain the reads NA of the allele A and the sequencing depth N at the site(s);

in some embodiments, allele A is a mutant-type gene, and the reads NA of allele A refers to the reads of mutant-type allele A; allele B is a wild-type gene, and the reads NB of allele B refers to the reads of wild-type allele B; the sequencing depth N at the site is the sum of the reads NA of allele A and the reads NB of allele B; and in some embodiments, the fetal cell-free nucleic acid is obtained through the detection of cell-free nucleic acids in maternal peripheral blood, wherein the detection of the cell-free nucleic acids in maternal peripheral blood comprises the detections of the mother's own cell-free nucleic acid and the cell-free nucleic acid of the fetus;
(4) calculating the probability that a fetus may have a normal chromosome copy number or abnormal different copy numbers at each SNP site; and calculating the probability values of the fetus being euploid or aneuploid, respectively, based on the percentage of mutant genotype in the cfDNA (A %) actually measured for each SNP site, the fetal fraction (ff) of cell-free nucleic acids and the mother's genotype at the site; wherein the maximum value among the sums of the probabilities at all valid SNP sites in the same chromosome is the interpreted karyotype of the fetus;
in some embodiments, the valid SNP sites are all the SNP sites where the genotypes of the fetus and those of the mother are not completely the same;
the calculated fetal karyotype H includes: D (disomy), MI (maternal trisomy type I), MII (maternal trisomy type II), PI (paternal trisomy type I), PII (paternal trisomy type II), LM (maternal microdeletion) and LP (paternal microdeletion);
the karyotype probabilities of the fetus at each SNP site is obtained by taking logarithm of the linear combination of π-weighted conditional beta binomial distribution probabilities, and the calculation equation is as follows:

$$log ( p ( NAi , N , pAi , H ) ) = log ( {\sum }_{k} π k Beta - Binom ( pAi , N , α , β ) )$$

i is the i-th valid SNP site;
N is the sequencing depth at the SNP site; pAi is the expected value of the reads percentage of a mutant-type from the next generation sequencing (NGS) at different gene loci of euploid or aneuploid fetus; when the fetus has different karyotypes, pAi is of different genotypes at different loci H, and their expected values will vary from each other; pAi of specific different loci H is shown in Table 1;
α is a discrete parameter selected for pAi based on the actual value in sequencing; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β=α/pAi−α
calculation of the weighting coefficient πk is based on different karyotypes of the fetus:

$$π k = {\sum }_{PATk} p ( FET ) \times p ( PATk )$$

wherein PATk ϵ{AA, AB, BB}, p(PATk) is calculated according to the Hardy-Weinberg equation, and the allele frequencies at the SNP site are p:

p(AA)=p×p

p(AB)=2×p×(1−p)

p(BB)=(1−p)×(1−p)
in some embodiments, the allele frequency p at the SNP site comes from a public database, more in some embodiments is selected from the 1000 Genomes database;
p(FET) is the possible genotype of the fetus, which is affected by the genotypes of father and mother, when the fetus is euploid or aneuploid, p(FET) is calculated according to Mendel's Laws of Inheritance, as shown in Table 2;

(5) calculation of fetal chromosome copy number variation,
during sperm or egg production, if a certain chromosome under examination does not undergo meiotic homologous recombination, the calculation equation for the distribution difference between probabilities of an abnormal chromosome copy number and a normal chromosome copy number is as follows:

$$Δ L = {\sum }_{1}^{M} ( log ( L D i ) - log ( L H i ) )$$

Hϵ{MI, MII, PI, PII, LM, LP}
LD is the probability value at the site in the euploid karyotype;
LH is the probability value at the site in the aneuploid karyotype;
M is the number of valid SNP sites in the chromosome;
chromosomal aneuploidy is positive when ΔL is less than the detection threshold in Table 2; the detection threshold is determined by the detection results of pregnant women's plasma samples with known prenatal diagnosis results and artificial mixtures of positive and negative reference samples; the detection thresholds for negative samples and positive samples, specific to the different aneuploid types, are shown in Table 3; and the method is a detection method for chromosome copy number.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the operation (5) of the method is:

(5) calculation of fetal chromosome microdeletion/microduplication,
during sperm or egg production, if a certain chromosome under examination is partially deleted or partially duplicated, the calculation equation for the distribution difference between probabilities of an abnormal chromosome copy number and a normal chromosome copy number is as follows:

$$Δ L = min ( {\sum }_{b 1}^{b 2} ( log ( L D i ) - log ( L H i ) ) )$$

Hϵ{MI, MII, PI, PII, LM, LP}, 0<b1, b2<M
b1 and b2 are the starting and ending positions at which the chromosome undergoes microdeletion/microduplication, respectively;
chromosomal aneuploidy is positive when ΔL is less than a detection threshold; the detection threshold is determined by the detection results of pregnant women's plasma samples with known prenatal diagnosis results and artificial mixtures of positive and negative reference samples; the detection thresholds for negative samples and positive samples are shown in Table 3; and the method is a detection method for chromosome microdeletion/microduplication;

or (5) calculation of dominant monogenic variation,
dominant monogenic variation occur in regions where the mother is homozygous wild-type BB; the probability that the A reads are from the fetus is calculated based on the reads NA of A, the sequencing depth N at the site, and the fetal fraction ff of cell-free nucleic acids through a beta binomial distribution fitting, and the calculated probability is compared with the probability of systematic noise, wherein: at a certain locus, the probability that the fetus has paternal or de novo mutations when the mother is homozygous wild-type BB is:

ΔL=log(beta−binom(pAi,N,α,β1))

−log(beta−binom(e,N,α,β2))
in some embodiments, pAi=ff/2,

$$Δ L = log ( beta - binom ( \frac{ff}{2} , N , α , β 1 ) ) - log ( beta - binom ( e , N , α , β 2 ) )$$

N is the sequencing depth at the site;
ff is the fetal fraction of cell-free nucleic acids;
α is a discrete parameter selected based on the actually measured value of the paternal allele in the fetal cell-free DNA; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β1=2×α/ff−α;
e is the systematic error rate at the site, and the systematic error rate is the ratio of mutant genotypes at the site in known negative samples; a is an actually measured discrete parameter of systematic noise, and the range of α is determined to be 1000-5000; in some embodiments, the value of a is 1000, 2000, 3000, 4000, or 5000;

β2=α/e−α
when ΔL is greater than the detection threshold which is 1, the gene mutation is positive; and the method is a detection method for dominant monogenic variation.

The log used in methods and systems of the present disclosure represents the value of log base e, wherein log(x) represents the natural logarithm, and its base value is e.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, further comprising: one or more combinations of calculation of fetal chromosome copy number variation, calculation of fetal chromosome microdeletion/microduplication, and calculation of dominant monogenic variation;

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, comprising calculation of fetal chromosome copy number variation; or calculation of fetal chromosome microdeletion/microduplication; or calculation of dominant monogenic variation; or calculation of fetal chromosome copy number variation and calculation of fetal chromosome microdeletion/microduplication; or calculation of fetal chromosome copy number variation and calculation of dominant monogenic variation; or calculation of fetal chromosome microdeletion/microduplication and calculation of dominant monogenic variation; or calculation of fetal chromosome copy number variation, calculation of fetal chromosome microdeletion/microduplication and calculation of dominant monogenic variation.

In the method, the detected gene mutation is only an intermediate result, and it cannot directly determine whether the fetus has a specific disease. For gene mutations that meet the detection threshold, further clinical data interpretation is required. Therefore, the detection method for chromosome copy number variation, chromosome microdeletion/microduplication, and/or dominant monogenic variation provided by methods and systems of the present disclosure may not be used for disease diagnosis, and is for non-diagnostic purposes.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein methods and systems of the present disclosure have no limitation on the method for calculating the fetal fraction (ff) of cell-free nucleic acids, and the detection and calculation can be carried out by any method well-known to those of ordinary skill in the art.

In some embodiments, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the operation (1) detects and calculates the fetal fraction (ff) of cell-free nucleic acids, and comprises the following operations:
when the mother is homozygous wild-type BB, the genotype of the fetus may be BB or BA, thus for the sites where the fetus is BA, the ratio distribution of reads A is centered on ff/2, and the fetal fraction of cell-free nucleic acids can be calculated by the median value ffBB of the ratio of reads A for all sites of this type; when the mother is homozygous mutant-type AA, the genotype of the fetus may be AA or AB, thus for the sites where the fetus is AB, the ratio distribution of reads A is centered on ff/2, and the fetal fraction of cell-free nucleic acids can be calculated by the median value ffAA of the ratio of reads B for all sites of this type; the fetal fraction (ff) of cell-free nucleic acids is calculated as follows:

ff=(ffAA+ffBB)/2
in some embodiments, when detecting and calculating the fetal fraction of cell-free nucleic acids, any chromosome site can be selected;
more in some embodiments, sites in the human genome where the copy number rarely changes are selected; further in some embodiments, sites in the human genome where the copy number rarely changes are selected; and these sites include or does not include sites in chromosomes 13, 18, 21, 22, X and Y.

In some embodiments, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the SNP site to be detected is one or more SNP sites selected from the chromosome to be detected, and is one or more of all chromosomes containing SNP sites; in some embodiments, the SNP site to be detected is one or more of chromosomes 13, 18, 21, 22, X and Y.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the equations for the sum of the probabilities at the chromosomal SNP sites in the case where one chromosomal recombination may occur during the production of parental germ cells are:

$$Δ L = min ( {\sum }_{1}^{k} ( log ( LDi ) - log ( LH 1 i ) ) + {\sum }_{k + 1}^{M} ( log ( LDi ) - log ( LH 2 i ) ) ) \\ Δ L = min ( {\sum }_{1}^{k} ( log ( LDi ) - log ( LH 2 i ) ) + {\sum }_{k + 1}^{M} ( log ( LDi ) - log ( LH 1 i ) ) )$$

H1, H2ϵ{MI, MII, PI, PII}; chromosomal aneuploidy is positive when one of the above two calculation results is less than the detection threshold in Table 3; and the detection thresholds for negative samples and positive samples are shown in Table 3.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the equations for the sum of the probabilities at the chromosomal SNP sites in the case where one or two chromosomal recombinations may occur during the production of parental germ cells are:

$$Δ L ( H 121 ) = min ( {\sum }_{1}^{b 1} ( log ( LDi ) - log ( LH 1 i ) ) + {\sum }_{b 1}^{b 2} ( log ( LDi ) - log ( LH 2 i ) ) + {\sum }_{b 2}^{M} ( log ( LDi ) - log ( LH 1 i ) ) \\ Δ L ( H 212 ) = min ( {\sum }_{1}^{b 1} ( log ( LDi ) - log ( LH 2 i ) ) + {\sum }_{b 1}^{b 2} ( log ( LDi ) - log ( LH 1 i ) ) + {\sum }_{b 2}^{M} ( log ( LDi ) - log ( LH 2 i ) ) )$$

H1, H2ϵ{MI, MII, PI, PII},
b1 and b2 are the calculated positions where the chromosome recombinations occur; chromosomal aneuploidy is positive when one of the above two calculation results is less than the detection threshold; and the detection thresholds for negative samples and positive samples are shown in Table 3.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the targeted capture probe covers all genes containing gene mutations; in some embodiments, the targeted capture probe covers the following genes: FGFR3, FGFR2, PTPN11, RAF1, RITZ, SOS1, COL1A1, COL1A2, COL2A1, OTC and MECP2.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the selection of one or more SNP sites in the chromosome to be detected is to prioritize sites with a simple structure and a GC content close to 40-60% based on the human genome sequence assembly build hg38.

In some embodiments, based on 1000G and gnomAD public databases, the sites having an allele frequency close to 0.3 to 0.7 are selected, and these sites include a total of at least 2320 SNP sites in chromosomes 1 to 22, X and Y.

The URLs of the public databases used are as below:
Human genome hg38:
hgdownload.cse.ucsc.edu/goldenpath/hg38/chromosomes/1000G:
www.internationalgenome.org/data/gnomAD:
gnomad.broadinstitute.org/

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the targeted capture probe used in the operation (3) is obtained using the following method of designing a targeted capture probe and the method comprises the following operations:
(1) determining the SNP site of interest;
(2) for each SNP site of targeted capture, designing four probes based on the SNP site, wherein the four probes are designed as -A-, -G-, -C-, -T- at the SNP site, respectively; and
(3) for each SNP site of targeted capture, calculating the annealing temperatures (Tm) for the binding of the four probes to two target sequences, respectively, wherein the two target sequences each carry two different single nucleotide polymorphisms; calculating the difference in annealing temperatures (ΔTm) for the binding of the four probes to the two target sequences based on the annealing temperature (Tm); and based on the calculation results, selecting the probe with the lowest ΔTm among the four probes and determining it as the optimal probe for the site.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein in the method of designing a targeted capture probe, the two target sequences are used as a reference gene sequence of the wild-type and a mutant gene sequence of the mutant-type, respectively; wherein the Tm values for the binding of the four probes to the reference gene sequence of the wild-type are: Tma, Tmg, Tmc, and Tmt, respectively, the Tm values for the binding of the four probes to the mutant gene sequence of the mutant-type are: Tma′, Tmg′, Tmc′, and Tmt′, respectively, and the ΔTm values for the binding of the four probes to the two target sequences are: |Tma−Tma′|, |Tmg−Tmg′|, |Tmc−Tmc′|, and |Tmt−Tmt′|, respectively.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein in the method of designing a targeted capture probe, the annealing temperature (Tm) for the probes is calculated using a nearest neighbor model and cation correction, and the calculation equation for the annealing temperature (Tm) for the probes is as below:

$${T}_{m} = \frac{Δ H}{Δ S + R \times ln {C}_{T}} + 16.6 log [ {Na}^{+} ]$$

ΔH represents the sum of standard enthalpy changes for all adjacent base pairs, ΔS represents the sum of standard entropy changes for all adjacent base pairs, R is the molar gas constant, CT represents the concentration of the primers, and [Na+] represents the concentration of monovalent sodium ions in solution.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein in the method of designing a targeted capture probe, the operation (2) is for each SNP site of targeted capture, designing four probes based on the SNP site, wherein the four probes are designed as -A-, -G-, -C-, -T- at the SNP site, respectively, and the rest positions are complementary to the sequence of interest.

In one embodiment, provided herein is a detection method for chromosome copy number variation, chromosome microdeletion/microduplication, and/or dominant monogenic variation, or use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the probe has a length of 100-200 bp; in some embodiments, the probe has a length of 100-190 bp or 100-180 bp or 100-170 bp or 100-160 bp or 100-150 bp or 100-140 bp or 100-130 bp or 100-120 bp or 110-200 bp or 110-190 bp or 110-180 bp or 110-170 bp or 110-160 bp or 110-150 bp or 110-140 bp or 110-130 bp or 110-120 bp; further, the probe has a length of 100 bp, 110 bp, 120 bp, 130 bp, 140 bp, 150 bp, 160 bp, 170 bp, 180 bp, 190 bp or 200 bp.

In some embodiments, provided herein is a method of designing a targeted capture probe for non-invasive prenatal screening of fetuses, comprising the following operations:
(1) determining the SNP site of interest;
(2) for each SNP site of targeted capture, designing four probes based on the SNP site, wherein the four probes are designed as -A-, -G-, -C-, -T- at the SNP site, respectively; and
(3) for each SNP site of targeted capture, calculating the annealing temperature (Tm) for the binding of the four probes to two target sequences, respectively, wherein the two target sequences each carry two different single nucleotide polymorphisms; calculating the difference in annealing temperatures (ΔTm) for the binding of the four probes to the two target sequences based on the annealing temperature (Tm); and based on the calculation results, selecting the probe with the lowest ΔTm among the four probes and determining it as the optimal probe for the site.

In one embodiment, provided herein is a method of designing a targeted capture probe for non-invasive prenatal screening of fetuses, wherein the two target sequences are used as a reference gene sequence of the wild-type and a mutant gene sequence of the mutant-type, respectively; wherein the Tm values for the binding of the four probes to the reference gene sequence of the wild-type are: Tma, Tmg, Tmc, and Tmt, respectively, the Tm values for the binding of the four probes to the mutant gene sequence of the mutant-type are: Tma′, Tmg′, Tmc′, and Tmt′, respectively, and the ΔTm values for the binding of the four probes to the two target sequences are: |Tma−Tma′|, |Tmg−Tmg′|, |Tmc−Tmc′|, and |Tmt−Tmf|, respectively.

In one embodiment, provided herein is a method of designing a targeted capture probe for non-invasive prenatal screening of fetuses, wherein the annealing temperature (Tm) for the probes is calculated using a nearest neighbor model and cation correction, and the calculation equation for the annealing temperature (Tm) for the probes is as below:

$${T}_{m} = \frac{Δ H}{Δ S + R \times ln {C}_{T}} + 16.6 log [ {Na}^{+} ]$$

ΔH represents the sum of standard enthalpy changes for all adjacent base pairs, ΔS represents the sum of standard entropy changes for all adjacent base pairs, R is the molar gas constant, CT represents the concentration of the primers, and [Na+] represents the concentration of monovalent sodium ions in solution.

In one embodiment, provided herein is a method of designing a targeted capture probe for non-invasive prenatal screening of fetuses, wherein the operation (2) is for each SNP site of targeted capture, designing four probes based on the SNP site, wherein the four probes are designed as -A-, -G-, -C-, -T- at the SNP site, respectively, and the rest positions are complementary to the sequence of interest.

In one embodiment, provided herein is a method of designing a targeted capture probe for non-invasive prenatal screening of fetuses, wherein the selection of the probe with the lowest ΔTm from the four probes is to select the probe with the lowest ΔTm for the reference gene sequence as the wild-type and the mutant gene sequence as the mutant-type.

In one embodiment, provided herein is a method of designing a targeted capture probe for non-invasive prenatal screening of fetuses, wherein the targeted capture probe covers all genes containing gene mutations; in some embodiments, the targeted capture probe covers the following genes: FGFR3, FGFR2, PTPN11, RAF1, RIT1, SOS1, COL1A1, COL1A2, COL2A1, OTC and MECP2, and the targeted capture probe is prepared using the method of designing a targeted capture probe for non-invasive prenatal screening of fetuses.

In one embodiment, provided herein is a method of designing a targeted capture probe for non-invasive prenatal screening of fetuses, wherein the probe has a length of 100-200 bp; in some embodiments, the probe has a length of 100-190 bp or 100-180 bp or 100-170 bp or 100-160 bp or 100-150 bp or 100-140 bp or 100-130 bp or 100-120 bp or 110-200 bp or 110-190 bp or 110-180 bp or 110-170 bp or 110-160 bp or 110-150 bp or 110-140 bp or 110-130 bp or 110-120 bp; further, the probe has a length of 100 bp, 110 bp, 120 bp, 130 bp, 140 bp, 150 bp, 160 bp, 170 bp, 180 bp, 190 bp or 200 bp.

In some embodiments, provided herein is a detection kit for non-invasive prenatal screening of fetuses, the kit comprising: the targeted capture probe for the one or more SNP sites used in the detection method for non-invasive prenatal screening of fetuses, and/or the targeted capture probe prepared using the method of designing a targeted capture probe for non-invasive prenatal screening of fetuses.

In one embodiment, provided herein is a detection kit for non-invasive prenatal screening of fetuses, wherein targeted capture probe covers all genes containing gene mutations; in some embodiments, the targeted capture probe covers the following genes: FGFR3, FGFR2, PTPN11, RAF1, RIT1, SOS1, COL1A1, COL1A2, COL2A1, OTC and MECP2, and the targeted capture probe is prepared using the method of designing a targeted capture probe for non-invasive prenatal screening of fetuses.

In another embodiment, provided herein is a detection kit for non-invasive prenatal screening of fetuses, wherein the probe has a length of 100-200 bp; in some embodiments, the probe has a length of 100-190 bp or 100-180 bp or 100-170 bp or 100-160 bp or 100-150 bp or 100-140 bp or 100-130 bp or 100-120 bp or 110-200 bp or 110-190 bp or 110-180 bp or 110-170 bp or 110-160 bp or 110-150 bp or 110-140 bp or 110-130 bp or 110-120 bp; further, the probe has a length of 100 bp, 110 bp, 120 bp, 130 bp, 140 bp, 150 bp, 160 bp, 170 bp, 180 bp, 190 bp or 200 bp.

In some embodiments, provided herein is a device for non-invasive prenatal screening of fetuses, comprising:
one or more processors; and
a memory for storing one or more programs;
when the one or more programs are executed by the one or more processors, the one or more processors are enabled to complete the detection method for non-invasive prenatal screening of fetuses or the detection method for chromosome copy number variation, chromosome microdeletion/microduplication, and/or dominant monogenic variation.

The sixth aspect of the present disclosure provides a computer-readable storage medium for non-invasive prenatal screening of fetuses with a computer program stored therein, wherein the program completes the detection method for non-invasive prenatal screening of fetuses or the detection method for chromosome copy number variation, chromosome microdeletion/microduplication, and/or dominant monogenic variation, when executed by a processor.

The seventh aspect of the present disclosure provides a system for non-invasive prenatal screening of fetuses, comprising a detection unit and an analysis unit, wherein the detection unit is used for:
detecting cell-free nucleic acids in maternal peripheral blood; in some embodiments, wherein the detection of the cell-free nucleic acids in maternal peripheral blood comprises the detections of the mother's own cell-free nucleic acid and the cell-free nucleic acid of the fetus;
using a targeted capture probe for one or more SNP sites to capture cell-free DNA in maternal peripheral blood, and then sequencing the cell-free DNA after amplification to obtain the sequencing result at the site(s), including the reads NA of allele A and the sequencing depth N at the site(s);
in some embodiments, allele A is a mutant-type gene, and the reads NA of allele A refers to the reads of mutant-type allele A; allele B is a wild-type gene, and the reads NB of allele B refers to the reads of wild-type allele B; the sequencing depth N at the site is the sum of the reads NA of allele A and the reads NB of allele B;
the analysis unit is used for:
calculating the probability that a fetus may have a normal chromosome copy number or abnormal different copy number at each SNP site; and calculating the probability values of the fetus being euploid or aneuploid, respectively, based on the percentage of mutant genotype in the cfDNA (A %) actually measured for each SNP site, the fetal fraction (ff) of cell-free nucleic acids and the mother's genotype at the site; wherein the maximum value among the sums of the probabilities at all valid SNP sites in the same chromosome is the interpreted karyotype of the fetus;
wherein the calculated fetal karyotype H includes: D (disomy), MI (maternal trisomy type I), MII (maternal trisomy type II), PI (paternal trisomy type I), PII (paternal trisomy type II), LM (maternal microdeletion) and LP (paternal microdeletion);
the karyotype probabilities of the fetus at each SNP site is obtained by taking logarithm of the linear combination of π-weighted conditional beta binomial distribution probabilities, and the calculation equation is as follows:

$$log ( p ( NAi , N , pAi , H ) ) = log ( {\sum }_{k} π k Beta - Binom ( pAi , N , α , β ) )$$

i is the i-th valid SNP site;
N is the sequencing depth at the SNP site; pAi is the expected value of the reads percentage of a mutant-type from the next generation sequencing (NGS) at different gene loci of euploid or aneuploid fetus; when the fetus has different karyotypes, pAi is of different genotypes at different loci H, and their expected values will vary from each other; the pAi of specific different loci H is shown in Table 1;
a is a discrete parameter selected for pAi based on the actual value in sequencing; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β=α/pAi−α
calculation of the weighting coefficient πk is based on different karyotypes of the fetus:

$$π k = {\sum }_{PATk} p ( FET ) \times p ( PATk )$$

wherein PATk ϵ{AA, AB, BB}, p(PATk) is calculated based on the Hardy-Weinberg equation, and the allele frequencies at the SNP site are p:

p(AA)=p×p

p(AB)=2×p×(1−p)

p(BB)=(1−p)×(1−p)
p(FET) is the possible genotype of the fetus, which is affected by the genotypes of father and mother, when the fetus is euploid or aneuploid, p(FET) is calculated according to Mendel's Laws of Inheritance, as shown in Table 2.

In one embodiment, provided herein is a system for non-invasive prenatal screening of fetuses, wherein the analysis unit is further used for calculation of fetal chromosome copy number variation, calculation of fetal chromosome microdeletion/microduplication, and/or calculation of dominant monogenic variation, wherein the calculation of fetal chromosome copy number variation is as follows: during sperm or egg production, if a certain chromosome under examination does not undergo meiotic homologous recombination, the calculation equation for the distribution difference between probabilities of an abnormal chromosome copy number and a normal chromosome copy number is as follows:

$$Δ L = {\sum }_{1}^{M} ( log ( LDi ) - log ( LHi ) )$$

Hϵ{MI, MII, PI, PII, LM, LP}
LD is the probability value at the site in the euploid karyotype;
LH is the probability value at the site in the aneuploid karyotype;
M is the number of valid SNP sites in the chromosome;
chromosomal aneuploidy is positive when ΔL is less than a detection threshold; the detection threshold is determined by the detection results of pregnant women's plasma samples with known prenatal diagnosis results and artificial mixtures of positive and negative reference samples; and the detection thresholds for negative samples and positive samples, specific to the different aneuploid types, are shown in Table 3; the calculation of fetal chromosome microdeletion/microduplication is as follows:
during sperm or egg production, if a certain chromosome under examination is partially deleted or partially duplicated, the calculation equation for the distribution difference between probabilities of an abnormal chromosome copy number and a normal chromosome copy number is as follows:

$$Δ L = min ( {\sum }_{b 1}^{b 2} ( log ( LDi ) - log ( LHi ) ) )$$

Hϵ{MI, MII, PI, PII, LP}, 0<b1, b2<M
b1 and b2 are the starting and ending positions at which the chromosome undergoes microdeletion/microduplication, respectively;
chromosomal aneuploidy is positive when ΔL is less than a detection threshold; the detection threshold is determined by the detection results of pregnant women's plasma samples with known prenatal diagnosis results and artificial mixtures of positive and negative reference samples; and the detection thresholds for negative samples and positive samples are shown in Table 3;
the calculation of dominant monogenic variation is as follows:
dominant monogenic variation occur in regions where the mother is homozygous wild-type BB; the probability that the A reads are from the fetus is calculated based on the reads NA of A, the sequencing depth N at the site, and the fetal fraction ff of cell-free nucleic acids through a beta binomial distribution fitting, and the calculated probability is compared with the probability of systematic noise, wherein at a certain locus, the probability that the fetus has paternal or de novo mutations when the mother is homozygous wild-type BB is:

$$Δ L = log ( beta - binom ( \frac{ff}{2} , N , α , β 1 ) ) - log ( beta - binom ( e , N , α , β 2 ) )$$

N is the sequencing depth at the site;
ff is the fetal fraction of cell-free nucleic acids;
α is a discrete parameter selected based on the actually measured value of the paternal allele in the fetal cell-free DNA; the actually measured value will deviate from the expected value due to the influence of experimental conditions; the range of α is determined to be 1000-5000 by using pre-mixed mother-child paired reference substances or maternal plasma samples; in some embodiments, the value of a is 1000, 2000, 3000, 4000, or 5000;

β1=2×α/ff−α;
e is the systematic error rate at the site, and the systematic error rate is the ratio of mutant genotypes at the site in known negative samples; a is an actually measured discrete parameter of systematic noise, and the range of α is determined to be 1000-5000; in some embodiments, the value of a is 1000, 2000, 3000, 4000, or 5000;

β2=α/e−α
when ΔL is greater than the detection threshold which is 1, the gene mutation is positive.

In some embodiments, provided herein is a system for non-invasive prenatal screening of fetuses, wherein the analysis unit is further used for calculation of the fetal fraction (ff) of cell-free nucleic acids,
wherein
the calculation of the fetal fraction (ff) of cell-free nucleic acids is as follows:
when the mother is homozygous wild-type BB, the genotype of the fetus may be BB or BA, thus for the sites where the fetus is BA, the ratio distribution of reads A is centered on ff/2, and the fetal fraction of cell-free nucleic acids can be calculated by the median value ffBB of the ratio of reads A for all sites of this type; when the mother is homozygous mutant-type AA, the genotype of the fetus may be AA or AB, thus for the sites where the fetus is AB, the ratio distribution of reads A is centered on ff/2, and the fetal fraction of cell-free nucleic acids can be calculated by the median value ffAA of the ratio of reads B for all sites of this type; the fetal fraction (ff) of cell-free nucleic acids is calculated as:

ff=(ffAA+ffBB)/2
in some embodiments, when detecting and calculating the fetal fraction of cell-free nucleic acids, any chromosome site can be selected;
more in some embodiments, sites in the human genome where the copy number rarely changes are selected; further in some embodiments, sites in the human genome where the copy number rarely changes are selected; and these sites include or does not include sites in chromosomes 13, 18, 21, 22, X and Y.

In some embodiments, provided herein is a system for non-invasive prenatal screening of fetuses, wherein the SNP site to be detected is one or more SNP sites selected from the chromosome to be detected, and is one or more of all chromosomes containing SNP sites; in some embodiments, the SNP site to be detected is one or more of chromosomes 13, 18, 21, 22, X and Y.

In some embodiments, provided herein is a system for non-invasive prenatal screening of fetuses, wherein the analysis unit is further used for the calculation of the sum of the probabilities at the chromosomal SNP sites in the case where one chromosomal recombination may occur during the production of parental germ cells, wherein the equations for the sum of the probabilities at the chromosomal SNP sites:

$$Δ L = min ( {\sum }_{1}^{k} ( log ( LDi ) - log ( LH 1 i ) ) + {\sum }_{k + 1}^{M} ( log ( LDi ) - log ( LH 2 i ) ) ) \\ Δ L = min ( {\sum }_{1}^{k} ( log ( LDi ) - log ( LH 2 i ) ) + {\sum }_{k + 1}^{M} ( log ( LDi ) - log ( LH 1 i ) ) )$$

H1, H2ϵ{MI, MII, PI, PII}; chromosomal aneuploidy is positive when one of the above two calculation results is less than the detection threshold; and the detection thresholds for negative samples and positive samples are shown in Table 3.

In some embodiments, provided herein is a system for non-invasive prenatal screening of fetuses, wherein the analysis unit is further used for the calculation of the sum of the probabilities at the chromosomal SNP sites in the case where one or two chromosomal recombinations may occur during the production of parental germ cells, wherein the equations for the sum of the probabilities at the chromosomal SNP sites:

$$Δ L ( H 121 ) = min ( {\sum }_{1}^{b 1} ( log ( LDi ) - log ( LH 1 i ) ) + {\sum }_{b 1}^{b 2} ( log ( LDi ) - log ( LH 2 i ) ) + {\sum }_{b 2}^{M} ( log ( LDi ) - log ( LH 1 i ) ) \\ Δ L ( H 212 ) = min ( {\sum }_{1}^{b 1} ( log ( LDi ) - log ( LH 2 i ) ) + {\sum }_{b 1}^{b 2} ( log ( LDi ) - log ( LH 1 i ) ) + {{\sum }_{b 2}}^{M} ( log ( LDi ) - log ( LH 2 i ) ) )$$

H1, H2ϵ{MI, MII, PI, PII},
b1 and b2 are the calculated positions where the chromosome recombinations occur; chromosomal aneuploidy is positive when one of the above two calculation results is less than the detection threshold; and the detection thresholds for negative samples and positive samples are shown in Table 3.

In some embodiments, provided herein is a system for non-invasive prenatal screening of fetuses, wherein the detection unit comprises a targeted capture probe for the one or more SNP sites, and the targeted capture probe covers all genes containing gene mutations; in some embodiments, the targeted capture probe covers the following genes: FGFR3, FGFR2, PTPN11, RAF1, RITZ, SOS1, COL1A1, COL1A2, COL2A1, OTC and MECP2.

In some embodiments, provided herein is a system for non-invasive prenatal screening of fetuses, wherein the detection unit comprises a targeted capture probe for the one or more SNP sites, and the targeted capture probe covers all genes containing gene mutations; in some embodiments, the targeted capture probe covers the following genes: FGFR3, FGFR2, PTPN11, RAF1, RITZ, SOS1, COL1A1, COL1A2, COL2A1, OTC and MECP2, and the targeted capture probe is a targeted capture probe prepared using the method of designing a targeted capture probe for non-invasive prenatal screening of fetuses according to any ones.

In some embodiments, provided herein is a system for non-invasive prenatal screening of fetuses, wherein the detection unit comprises a targeted capture probe for the one or more SNP sites, wherein the probe has a length of 100-200 bp; in some embodiments, the probe has a length of 100-190 bp or 100-180 bp or 100-170 bp or 100-160 bp or 100-150 bp or 100-140 bp or 100-130 bp or 100-120 bp or 110-200 bp or 110-190 bp or 110-180 bp or 110-170 bp or 110-160 bp or 110-150 bp or 110-140 bp or 110-130 bp or 110-120 bp; further, the probe has a length of 100 bp, 110 bp, 120 bp, 130 bp, 140 bp, 150 bp, 160 bp, 170 bp, 180 bp, 190 bp or 200 bp.

The eighth aspect of the present disclosure provides use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the targeted capture probe is a targeted capture probe for the one or more SNP sites;

in some embodiments, the targeted capture probe is a targeted capture probe prepared using the method of designing a targeted capture probe for non-invasive prenatal screening of fetuses according to any ones;

more in some embodiments, the targeted capture probe covers all genes containing gene mutations; in some embodiments, the targeted capture probe covers the following genes: FGFR3, FGFR2, PTPN11, RAF1, RIT1, SOS1, COL1A1, COL1A2, COL2A1, OTC and MECP2, and the targeted capture probe is prepared using the method of designing a targeted capture probe for non-invasive prenatal screening of fetuses according to any ones.

In another embodiment, provided herein is use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the targeted capture probe is a targeted capture probe for the one or more SNP sites, wherein the probe has a length of 100-200 bp; in some embodiments, the probe has a length of 100-190 bp or 100-180 bp or 100-170 bp or 100-160 bp or 100-150 bp or 100-140 bp or 100-130 bp or 100-120 bp or 110-200 bp or 110-190 bp or 110-180 bp or 110-170 bp or 110-160 bp or 110-150 bp or 110-140 bp or 110-130 bp or 110-120 bp; further, the probe has a length of 100 bp, 110 bp, 120 bp, 130 bp, 140 bp, 150 bp, 160 bp, 170 bp, 180 bp, 190 bp or 200 bp.

In some embodiments, provided herein is use of a targeted capture probe in the preparation of reagents or kits for performing non-invasive prenatal screening of fetuses, or use of a targeted capture probe for non-invasive prenatal screening of fetuses, or a targeted capture probe for non-invasive prenatal screening of fetuses, wherein the method for non-invasive prenatal screening of fetuses comprises: part or all of the operations of the detection method for non-invasive prenatal screening of fetuses provided by the first aspect, or part or all of the operations of the detection method for chromosome copy number variation, chromosome microdeletion/microduplication, and/or dominant monogenic variation by the second aspect.

In the present disclosure, the nucleotide sequence of a polynucleotide having at least 90% “identity” to a reference nucleotide sequence generally indicates that in each 100 nucleotides of the reference nucleotide sequence, the nucleotide sequence of the polynucleotide is the same as the reference sequence besides up to 10 nucleotides. In other words, to obtain a polynucleotide whose nucleotide sequence has at least 90% identity to a reference nucleotide sequence, up to 10% nucleotides in the reference sequence can be replaced by other nucleotides or deleted; or some nucleotides can be inserted into the reference sequence, wherein the inserted nucleotides can reach up to 10% of total nucleotides of the reference sequence; or in some polynucleotides, there is a combination of deletion, insertion and substitution, wherein the deleted or inserted and substituted nucleotides are up to 10% of total nucleotides of the reference sequence. These deletions, insertions and substitutions of the reference sequence can take place in 5′ or 3′ end position of the reference nucleotide sequence, or any positions therebetween, and they may be separately distributed in the nucleotides of the reference sequence, or present in the reference sequence in forms of one or more adjacent combinations.

In the present disclosure, algorithms for determining percent sequence identity and sequence similarity include for example BLAST and BLAST 2.0 algorithms. BLAST and BLAST 2.0 can be used for determining percent sequence identity of the nucleotide sequences. Software for BLAST analysis can be publically acquired from National Center for Biotechnology Information (NCBI).

In the present disclosure, the nucleotide sequence having at least 90% sequence identity to the nucleotide sequence of the reference sequence includes a polynucleotide sequence which is basically identical to the sequence disclosed in reference sequence, for example those sequences having at least 90% sequence identity, in some embodiments at least 91%, 92%, 93%, 94%, 95%, 96%, 97%, 98% or 99% or more sequence identity to the polynucleotide sequence, for example, as determined by the method (for example BLAST analysis using standard parameters).

In some embodiments, “hybridization conditions” are classified according to “stringency” degree of the condition used when hybridization is measured. The stringency degree can be based on for example a melting temperature (Tm) of a nucleic acid binding composite or probe. For example, “highest stringency” may occur at about Tm-5° C. (5° C. below probe Tm); “higher stringency” occurs at about 5-10° C. below Tm; “moderate stringency” occurs at about 10-20° C. below probe Tm; and “low stringency” occurs at about 20-25° C. below Tm. Alternatively, or further, the hybridization conditions can be based on the salt or ion strength conditions and/or one or more stringency washing of the hybridization. For example, 6×SSC=extremely low stringency; 3×SSC=low to moderate stringency; 1×SSC=moderate stringency; and 0.5×SSC=higher stringency. Functionally, the highest stringency condition can be used to determine a nucleic sequence that is stringently identical or nearly stringently identical to the hybridization probe; and the higher stringency condition is used to determine a nucleic acid sequence that has about 80% or more sequence identity to this probe.

For applications requiring high selectivity, relatively stringent conditions may be used to form a hybrid, for example, selecting a relatively low salt and/or high-temperature condition. Hybridization conditions including moderate stringency and higher stringency are provided in Sambrook et al. (Sambrook, J. et al. (1989) Molecular Cloning, Laboratory Manual, Cold Spring Harbor Press, Plainview, N.Y.) ISBN-10 0-87969-577-3.

For the convenience of explanation, the proper moderate stringency conditions for detecting the hybridization of the polynucleotide, and other polynucleotides include: pre-washing with 5×SSC, 0.5% SDS, 1.0 mM EDTA (PH8.0) solution; hybridizing for overnight in 5×SSC at 50-65° C.; and subsequently washing twice for 20 min respectively at 65° C. with 2×, 0.5× and 0.2×SSC containing 0.1% SDS. It should be understood by those skilled in the art that hybridization stringency can be easily manipulated, for example, the salt content of the hybridization solution and/or hybridization temperature can be changed. For example, in another embodiment, the proper higher stringency hybridization conditions include the above conditions, except that the hybridization temperature is raised to for example 60-65° C. or 65-70° C.

### Computer System

Any of the methods disclosed herein can be performed and/or controlled by one or more computer systems. In some examples, any operation of the methods disclosed herein can be wholly, individually, or sequentially performed and/or controlled by one or more computer systems. Any of the computer systems mentioned herein can utilize any suitable number of subsystems. In some embodiments, a computer system includes a single computer apparatus, where the subsystems can be the components of the computer apparatus. In other embodiments, a computer system can include multiple computer apparatuses, each being a subsystem, with internal components. A computer system can include desktop and laptop computers, tablets, mobile phones and other mobile devices.

The subsystems can be interconnected via a system bus. Additional subsystems include a printer, keyboard, storage device(s), and monitor that is coupled to display adapter. Peripherals and input/output (I/O) devices, which couple to I/O controller, can be connected to the computer system by any number of connections known in the art such as an input/output (I/O) port (e.g., USB, FireWire®). For example, an I/O port or external interface (e.g., Ethernet, Wi-Fi, etc.) can be used to connect computer system to a wide area network such as the Internet, a mouse input device, or a scanner. The interconnection via system bus allows the central processor to communicate with each subsystem and to control the execution of a plurality of instructions from system memory or the storage device(s) (e.g., a fixed disk, such as a hard drive, or optical disk), as well as the exchange of information between subsystems. The system memory and/or the storage device(s) can embody a computer readable medium. Another subsystem is a data collection device, such as a camera, microphone, accelerometer, and the like. Any of the data mentioned herein can be output from one component to another component and can be output to the user.

A computer system can include a plurality of the same components or subsystems, e.g., connected together by external interface or by an internal interface. In some embodiments, computer systems, subsystem, or apparatuses can communicate over a network. In such instances, one computer can be considered a client and another computer a server, where each can be part of a same computer system. A client and a server can each include multiple systems, subsystems, or components.

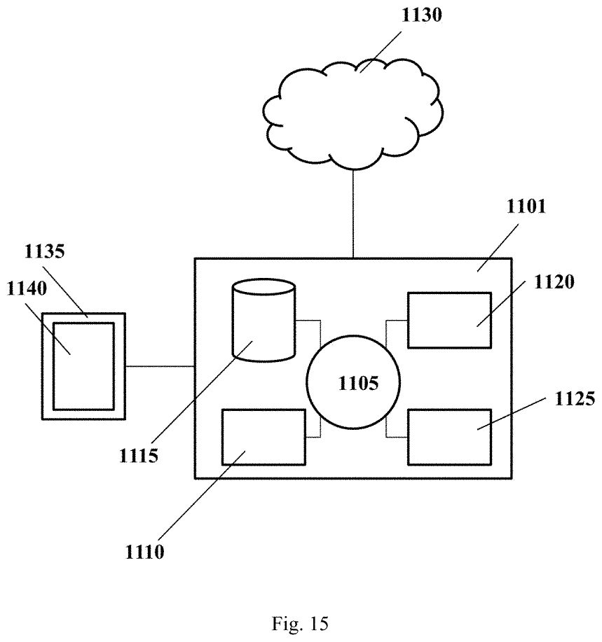

The present disclosure provides computer control systems that are programmed to implement methods of the disclosure for analyzing nucleic acid molecules. FIG. 15 shows a computer system 1101 that is programmed or otherwise configured to analyze nucleic acid molecules or sequence reads thereof as described herein. The computer system 1101 can implement and/or regulate various aspects of the methods provided in the present disclosure, such as, for example, controlling sequencing of the nucleic acid molecules from a biological sample, performing various operations of the bioinformatics analyses of sequencing data as described herein, integrating data collection, analysis and result reporting, and data management. The computer system 1101 can be an electronic device of a user or a computer system that is remotely located with respect to the electronic device. The electronic device can be a mobile electronic device.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The storage unit 1115 can store files, such as drivers, libraries and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a Smart phone installed with application that receives and displays results of sample analysis sent from the computer system 1101). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that includes a user interface (UI) 1140 for providing, for example, results of sample analysis, such as, but not limited to graphic showings of pathogen integration profile, genomic location of pathogen integration breakpoints, classification of pathology (e.g., type of disease or cancer and level of cancer), and treatment suggestion or recommendation of preventive operations based on the classification of pathology. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, control sequencing of the nucleic acid molecules from a sample, direct collection of sequencing data, analyzing the sequencing data, performing SNP-based analysis, detecting the presence or absence of chromosomal aneuploidy or monogenic variation, or generating a report of the detection results.

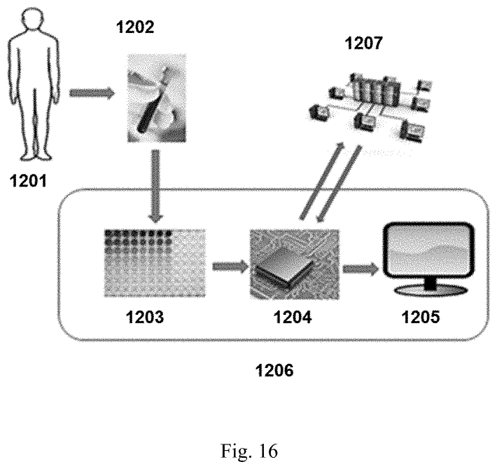

In some cases, as shown in FIG. 16, a sample 1202 may be obtained from a subject 1201, such as a human subject. A sample 1202 may be subjected to one or more methods as described herein, such as subjected to amplification, probe capturing, and/or sequencing. One or more results from a method may be input into a processor 1204. One or more input parameters such as a sample identification, subject identification, sample type, a reference, or other information may be input into a processor 1204. One or more metrics from an assay may be input into a processor 1204 such that the processor may produce a result, such as a classification of pathology (e.g., diagnosis) or a recommendation for a treatment. A processor may send a result, an input parameter, a metric, a reference, or any combination thereof to a display 1205, such as a visual display or graphical user interface. A processor 1204 may (i) send a result, an input parameter, a metric, or any combination thereof to a server 1207, (ii) receive a result, an input parameter, a metric, or any combination thereof from a server 1207, (iii) or a combination thereof.

Aspects of the present disclosure can be implemented in the form of control logic using hardware (e.g., an application specific integrated circuit or field programmable gate array) and/or using computer software with a generally programmable processor in a modular or integrated manner. As used herein, a processor includes a single-core processor, multi-core processor on a same integrated chip, or multiple processing units on a single circuit board or networked. Based on the disclosure and teachings provided herein, a person of ordinary skill in the art will know and appreciate other ways and/or methods to implement embodiments described herein using hardware and a combination of hardware and software.

Any of the software components or functions described in this application can be implemented as software code to be executed by a processor using any suitable computer language such as, for example, Java, C, C++, C#, Objective-C, Swift, or scripting language such as Perl or Python using, for example, conventional or object-oriented techniques. The software code can be stored as a series of instructions or commands on a computer readable medium for storage and/or transmission. A suitable non-transitory computer readable medium can include random access memory (RAM), a read only memory (ROM), a magnetic medium such as a hard-drive or a floppy disk, or an optical medium such as a compact disk (CD) or DVD (digital versatile disk), flash memory, and the like. The computer readable medium can be any combination of such storage or transmission devices.

Such programs can also be encoded and transmitted using carrier signals adapted for transmission via wired, optical, and/or wireless networks conforming to a variety of protocols, including the Internet. As such, a computer readable medium can be created using a data signal encoded with such programs. Computer readable media encoded with the program code can be packaged with a compatible device or provided separately from other devices (e.g., via Internet download). Any such computer readable medium can reside on or within a single computer product (e.g., a hard drive, a CD, or an entire computer system), and can be present on or within different computer products within a system or network. A computer system can include a monitor, printer, or other suitable display for providing any of the results mentioned herein to a user.

Any of the methods described herein can be totally or partially performed with a computer system including one or more processors, which can be configured to perform the operations. Thus, embodiments can be directed to computer systems configured to perform the operations of any of the methods described herein, with different components performing a respective operations or a respective group of operations. Although presented as numbered operations, operations of methods herein can be performed at a same time or in a different order. Additionally, portions of these operations can be used with portions of other operations from other methods. Also, all or portions of an operation can be optional. Additionally, any of the operations of any of the methods can be performed with modules, units, circuits, or other approaches for performing these operations.

### Other Embodiments

The section headings used herein are for organizational purposes only and are not to be construed as limiting the subject matter described.

It is to be understood that the methods described herein are not limited to the particular methodology, protocols, subjects, and sequencing techniques described herein and as such can vary. It is also to be understood that the terminology used herein is for the purpose of describing particular embodiments only, and is not intended to limit the scope of the methods and compositions described herein, which will be limited only by the appended claims. While some embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein can be employed in practicing the disclosure. It is intended that the following claims define the scope of the disclosure and that methods and structures within the scope of these claims and their equivalents be covered thereby.

Several aspects are described with reference to example applications for illustration. Unless otherwise indicated, any embodiment can be combined with any other embodiment. It should be understood that numerous specific details, relationships, and methods are set forth to provide a full understanding of the features described herein. A skilled artisan, however, will readily recognize that the features described herein can be practiced without one or more of the specific details or with other methods. The features described herein are not limited by the illustrated ordering of acts or events, as some acts can occur in different orders and/or concurrently with other acts or events. Furthermore, not all illustrated acts or events are required to implement a methodology in accordance with the features described herein.

### EXAMPLES

The following examples are given for the purpose of illustrating various embodiments of the invention and are not meant to limit the present disclosure in any fashion. The present examples, along with the methods described herein are presently representative of some embodiments, are exemplary, and are not intended as limitations on the scope of the invention. Changes therein and other uses which are encompassed within the spirit of the invention as defined by the scope of the claims will occur to those skilled in the art.

It is noted that in the following examples, unless otherwise mentioned, experiment methods without specific implementation conditions are typically exerted according to conventional conditions or conditions suggested by instrument manufacturers. Unless otherwise defined, the meanings of all the professional and scientific terms used herein are the same as those familiar with those skilled in the art.

### Example 1: Capture of DNA with the Target Probe

a. Separation of Plasma and Extraction of Cell-Free DNA

A blood collection tube was placed in a centrifugal machine to be centrifuged at 1600 g, centrifugation time: 10 min in EDTA anticoagulation tube, and 15 min in Streck tube. After centrifugation was completed, supernatant was slowly pipetted into a 5 mL transfer tube from top to bottom, and centrifuged again at 16000 g for 10 min. The purpose of secondary centrifugation of plasma is to remove all cellular contaminants. Cell-free DNA was extracted using a TIANGEN® Magnetic Serum/Plasma DNA Maxi Kit, including: treating plasma samples with proteinase K, carrying out a water bath for 20 min at 60° C., adding MagAttract Suspension E, Buffer GHH and Carrier RNA, uniformly mixing for 30 s by vortexing, and then incubating for 15 min at room temperature so that magnetic beads adsorbed the nucleic acids. The rinsing solution Buffer PWG was used, and uniformly mixed by vortexing, so that the magnetic beads were fully suspended. Finally, the nucleic acids were dissolved with eluant, and the eluant was collected and quantified by quality inspection.

b. Sequencing Library Construction

End repairing was performed on the cell-free DNA using End repair & A-tailing Buffer and End repair & A-tailing Enzyme. The reaction was performed according to the following conditions: 20° C. for 30 min; and 65° C. for 30 min. Linker addition reaction was performed using mADPta01 (15 μM), Ligation Buffer and DNA Ligase according to the following conditions: 20° C. for 15 min. PCR amplification and sequencing tag addition were performed using 2× HiFi PCR MasterMix and HS-mp101 (100 μM) and Index Primer (4 nmol) (100 μM). After PCR amplification, fragment screening, purification and recovery were performed using magnetic beads. The library was quantified using Qubit™ 1× dsDNA HS Assay Kit for quality inspection, which required cell-free DNA library of ≥500 ng. If this condition cannot be met, the library needs to be rebuilt. 34 of 2× Loading Buffer was added to 1 μL the library for electrophoresis for 30 min at the voltage of 120V to check whether electrophoretic bands were abnormal.

c. Enrichment, Library Hybridization, Library Elution, PCR Amplification, Library Purification and Library Quality Inspection

1. Enrichment

1.1 400 ng of each of NGS library samples obtained in operation b, or a plurality (such as 24) of different NGS library samples (here adaptive to samples generated by other NGS library preparation solutions) subjected to end repair, linker ligation and index+universal primer amplification were placed in a new 1.5 mL low-adsorption centrifugal tube and uniformly mixed, and a certain amount of mixed sample was taken as a reserved sample before hybridization capture and temporarily stored in a refrigerator at 4° C.

2. Library Hybridization

2.1 AMpure XP beads (abbreviation for XP magnetic beads in the following operations) were needed to be taken out in advance and balanced for 30 min at room temperature, and then uniformly mixed by vortexing for later use. 80% ethanol was freshly prepared according to usage amount.

2.2 Cot-1 DNA and XP magnetic beads were added into the 1.5 mL low-adsorption centrifugal tube containing the total library in operation 1.1 according to Table 4, uniformly mixed via blowing & suction, incubated for 10 min at room temperature and centrifuged transiently, then the centrifugal tube was placed on a magnetic frame for 5 min until the liquid was completely clear, and then supernatant was discarded.

TABLE 4

| Components | Addition amount |
|---|---|
| Mixed library | the amount of library prepared in |
| | operation 1.1. |
| Human Cot-1 DNA | 7.5 μL |
| XP magnetic beads | 1.8 times of a sum of the above two |
| | volumes |

Mixed library: the requirement of quality inspection should be met, and the quantification range of the volume of the mixed library was not set.

2.3 80% ethanol was slowly added along the wall of the tube on the magnetic frame to ensure immersion of XP magnetic beads and then stood for 30 s, and the supernatant was discarded.

2.4 Operation 2.3 was repeated once.

2.5 Transient centrifugation was performed, remnant ethanol was removed using a 10 μL micropipette, the centrifugal tube was put on a constant-temperature blending instrument which was heated to 37° C. in advance until 80% ethanol on the surface of XP magnetic beads was completely removed.

Note: ensure that XP magnetic beads will not be too dry or cracked.

2.6 Eluant was prepared in a 0.2 mL PCR tube according to Table 5, and uniformly mixed by vortexing.

TABLE 5

| Reagent name | Volume (μL) |
|---|---|
| XGen 2 × Hybridization Buffer | 9.5 |
| XGen Hybridization Buffer Enhancer | 3 |
| NanoPrep Blocker | 2 |
| Probe | 4.5 |
| Total volume | 19 |

2.7 194 of eluant as described above was added to the XP magnetic beads dried in operation 2.5, uniform mixed by blowing & suction, and placed for 5 min at room temperature. The above mixture was placed on the magnetic frame to stand for 2 min, and 174 of supernatant was transferred to a new 0.2 mL low-adsorption PCR tube and transiently centrifuged. The 0.2 mL PCR tube was placed on a gene amplification instrument to react under the conditions which were as follows: 95° C. for 30 s; 65° C. for 16 h; and 65° C. hold (hot cover temperature was 100° C.).

3. Library Elution

3.1 For single capture reaction, buffer was diluted, as shown in Table 6.

TABLE 6

| | Concentrated | NF |
|---|---|---|
| Reagent name | buffer (μL) | water (μL) |
| 2 × Bead Wash Buffer | 250 | 250 |
| 10 × Wash Buffer I * | 30 | 270 |
| 10 × Wash Buffer II | 20 | 180 |
| 10 × Wash Buffer III | 20 | 180 |
| 10 × Stringent Wash Buffer | 40 | 360 |

3.2 1× Wash Buffer I and 1× Stringent Wash Buffer after being diluted in operation 3.1 were subpackaged according to volumes in Table 7; buffers needing to be stored at 65° C. were put on the gene amplification instrument in operation 3.9, and other buffers were placed at room temperature.

TABLE 7

| | | Specification of | |
|---|---|---|---|
| | | centrifugal tube | Storage |
| Reagent name | Volume | (mL) | conditions |
| Wash Buffer I * | 100 μL | 0.2 | 65° C. |
| Wash Buffer I * | 200 μL | 1.5 | Room |
| | | | temperature |
| Stringent Wash Buffer | 200 μL | 0.2 | 65° C. |
| Stringent Wash Buffer | 200 μL | 0.2 | 65° C. |

3.3 Before use, M-270 magnetic beads were balanced for 30 min at room temperature and vortexed for 15 s to be completely uniformly mixed. 100 μL M-270 magnetic beads required for each capture were equally distributed into individual 1.5 mL low-adsorption centrifugal tubes. The 1.5 mL low-adsorption centrifugal tubes were placed on the magnetic frame to stand for 2 min so that M-270 magnetic beads were completely separated from supernatant. The supernatant was discarded and M-270 magnetic beads were ensured to be left in the tubes.

3.4 M-270 magnetic beads were washed: 2004, 1× Bead Wash Buffer was added into the above centrifugal tubes, vortexed for 10 s and transiently centrifuged, the centrifugal tubes were placed on the magnetic frame so that M-270 magnetic beads were completely separated from supernatant. The supernatant was discarded.

3.5 Operation 3.4 was repeated once.

3.6 100 μL of 1× Bead Wash Buffer was added into the above centrifugal tubes and uniformly mixed by blowing & suction.

3.7 100 μL of resuspended M-270 magnetic beads were respectively transferred to new 0.2 mL low-adsorption PCR tubes.

3.8 The 0.2 mL low-adsorption PCR tubes were put on the gene amplification instrument in which hybridization was completed and at a temperature of 65° C., and magnetic strips were placed close to the PCR tube, so that M-270 magnetic beads were completely separated from supernatant, and the supernatant was discarded.

Note: the following operation should be performed instantly.

3.9 The hybridization sample in operation 2.7 was transferred to 0.2 mL low-adsorption PCR tube in operation 3.8, and subjected to slight blowing & suction for 10 times using a micropipette so that the hybridization sample was thoroughly uniformly mixed (a 20 μL low-adsorption gun head was used in this operation). The above mixed sample was incubated for 45 min at 65° C. with slight blowing & suction for 10 times every 15 min, so as to ensure that M-270 magnetic beads were kept at a suspension state.

3.10 100 μL of 1× Wash Buffer I preheated at 65° C. was added into the tube in operation 3.9. The sample was uniformly mixed by slow blowing & suction for 10 times using a micropipette. Magnetic strips were placed close to the PCR tube so that M-270 magnetic beads were completely separated from supernatant, and the supernatant was discarded.

3.11 200 μL of 1× Stringent Wash Buffer preheated at 65° C. was added. The sample was uniformly mixed by slow blowing & suction for 10 times using a micropipette. Magnetic strips were placed close to the PCR tube so that M-270 magnetic beads were completely separated from supernatant, and the supernatant was discarded.

3.12 Operation 3.11 was repeated once.

3.13 200 μL of 1× Wash Buffer I placed at room temperature was added, uniformly mixed by blowing & suction, then transferred to a 1.5 mL low-adsorption centrifugal tube and vortexed for 2 min, the 1.5 mL low-adsorption centrifugal tube was put on the magnetic frame so that M-270 magnetic beads were completely separated from supernatant, and the supernatant was discarded.

3.14 200 μL of 1× Wash Buffer II at room temperature was added and vortexed for 1 min, the 1.5 mL low-adsorption centrifugal tube was put on the magnetic frame so that M-270 magnetic beads were completely separated from supernatant, and the supernatant was discarded.

3.15 200 μL of 1× Wash Buffer III at room temperature was added and vortexed for 30 s, the 1.5 mL low-adsorption centrifugal tube was put on the magnetic frame so that M-270 magnetic beads were completely separated from supernatant, and the supernatant was discarded.

3.16 The above 1.5 mL low-adsorption centrifugal tubes were taken away from the magnetic frame, and 20 μL of NF water was added to the M-270 magnetic beads and then uniformly mixed by blowing & suction so that the magnetic beads were resuspended. Ensure that any magnetic beads adhered to the side wall of the tube were all resuspended.

Note: the magnetic beads were not discarded, and 204 of entire suspended magnetic beads and the captured DNA library were used in operation 4.2.

4. PCR Amplification

4.1 2× HiFi PCR Master Mix and NanoPrepTMM-Amplificatiom Primer Mix were naturally molten on ice, and the NanoPrepTMM-Amplificatiom Primer Mix was uniformly mixed by vortexing, and transiently centrifuged for later use. A PCR reaction system was prepared in a 0.2 mL PCR tube placed on the ice according to Table 8 and uniformly mixed by vortexing, and transiently centrifuged.

Note: the magnetic beads with captured DNA were separately added, and uniformly mixed by blowing & suction.

TABLE 8

| Reagent name | Volume (μL) |
|---|---|
| 2 × HiFi PCR Master Mix | 25 |
| NanoPrepTMM- | 5 |
| Amplificatiom Primer Mix | |
| Magnetic beads with | 20 |
| captured DNA | |
| Total volume | 50 |

4.2 The 0.2 mL PCR tube was put in the gene amplification instrument, and the following procedures were operated under the condition that a heating cover was at 105° C.

TABLE 9

| | Cycle | Temperature | | |
|---|---|---|---|---|
| Operations | number | (° C.) | Duration | |
| | 1 | 98 | 45 | s |
| Denaturation | 12 | 98 | 15 | s |
| Annealing | | 60 | 30 | s |
| Extension | | 72 | 30 | s |
| | 1 | 72 | 1 | min |

| | | |
|---|---|---|
| 1 | 4 | ∞ |

5. Purification of Library

5.1 XP magnetic beads were needed to be taken out in advance, balanced for 30 min at room temperature, and then uniformly mixed by vortexing to be used, and 80% ethanol was freshly prepared according to usage amount.

5.2 The 0.2 mL PCR tube was taken out after amplification was ended, and transiently centrifuged. 504 of amplified products were transferred to a 1.5 mL low-adsorption centrifugal tube containing 75 μL of XP magnetic beads, and the centrifugal tube was subjected to point vibration for 10 times and stood for 10 min.

5.3 The centrifugal tube was placed on the magnetic frame for 5 min, the supernatant was discarded, 200 μL of 80% ethanol was added so as to immerse the XP magnetic beads, the centrifugal tube was subjected to standing for 30 s, and the supernatant was discarded.

5.4 Operation 5.3 was repeated.

5.5 The above centrifugal tube was transiently centrifuged, residual ethanol was removed using a 10 μL micropipette, and the centrifugal tube was put on a constant-temperature blending instrument heated to 37° C. in advance until 80% ethanol on the surface of XP magnetic beads was completely removed.

Note: ensure that the magnetic beads will not be too dry or cracked.

5.6 33 μL of NF water was added to the dried XP magnetic beads and uniform mixed by blowing & suction. The obtained mixture was placed for 2 min at room temperature, the centrifugal tube was placed on the magnetic frame for 2 min, and 30 μL of elution product was transferred to a new 1.5 mL low-adsorption centrifugal tube. Ensure that the elution product did not carry magnetic beads.

6. Quality Inspection of Library

6.1 Qubit quantification: the concentration of nucleic acid in 1 μL of library sample was correctly measured using Qubit™ dsDNA HS Assay Kit.

6.2 Electrophoresis detection: 20 ng of libraries before and after capture were taken respectively and diluted to 4 μL with water, and amplified using three pairs of primers namely P2, P3 and N2, respectively. Amplification systems are as shown in Table 10 below, and amplification procedures are as shown in Table 11 below.

TABLE 10

| Reagent name | | Volume (μL) |
|---|---|---|
| 2 × HiFi PCR Master Mix | | 2.5 |
| primer | F | 0.75 |
| | R | 0.75 |
| library to be detected | | 1 |
| volume of total system | | 5 |

TABLE 11

| | Cycle | Temperature | | |
|---|---|---|---|---|
| | number | (° C.) | Duration | |
| | 1 | 98 | 45 | s |
| Denaturation | 25 | 98 | 15 | s |
| Annealing | 1. | 58 | 15 | s |
| Extension | | 72 | 15 | s |
| | 1 | 72 | 1 | min |

| | | |
|---|---|---|
| 1 | 4 | ∞ |

After amplification was completed, 54, of 2× Loading Buffer was added to the product, and the above product was subjected to electrophoresis for 30 min at the voltage of 120V using 1.5% agarose gel. The comparative electrophoresis results were checked.

Result Analysis

1. The enrichment degrees of a target region before and after capture were compared. The PCR primers used for library hybridization and quality inspection are shown in Table 12. For DNA fragments in a non-capture region, the enrichment degrees before and after hybridization capture were the same. However, for DNA fragments in a capture region, the enrichment degree after hybridization capture was more than 10 times that before hybridization capture, which meets the quality inspection requirements (FIG. 1).

TABLE 12
PCR primers for library hybridization and quality inspection

| Primer | | | Size of |
|---|---|---|---|
| name | Forward sequence | Reverse sequence | amplicon |
| P2 | TGGGCTTCTTCCTGTTCATC | GGAAGCGGGAGATCTTGTG | 111 |
| | (SEQ ID NO: 1) | (SEQ ID NO: 2) | |
| P3 | AGCTGTCACCCACATCAAG | TTAATTGCCCGTGATGTTCC | 119 |
| | A | (SEQ ID NO: 4) | |
| | (SEQ ID NO: 3) | | |
| N2 | GGTTCATTAACCTGGGCTG | CTAGCCCCAAGTGAGACCT | 108 |
| | A | G | |
| | (SEQ ID NO: 5) | (SEQ ID NO: 6) | |

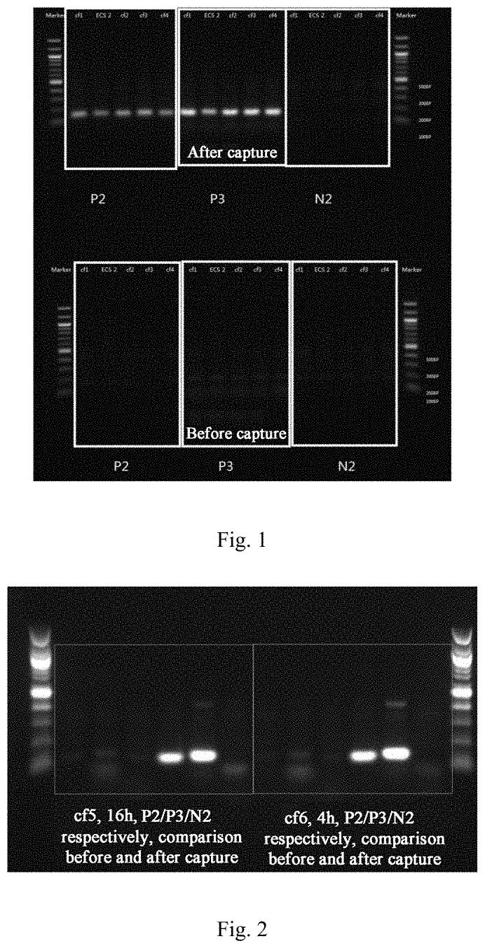

2. The capture efficiencies for the target region after hybridization capture for 4 h or 16 h are not obviously changed, as shown in FIG. 2.

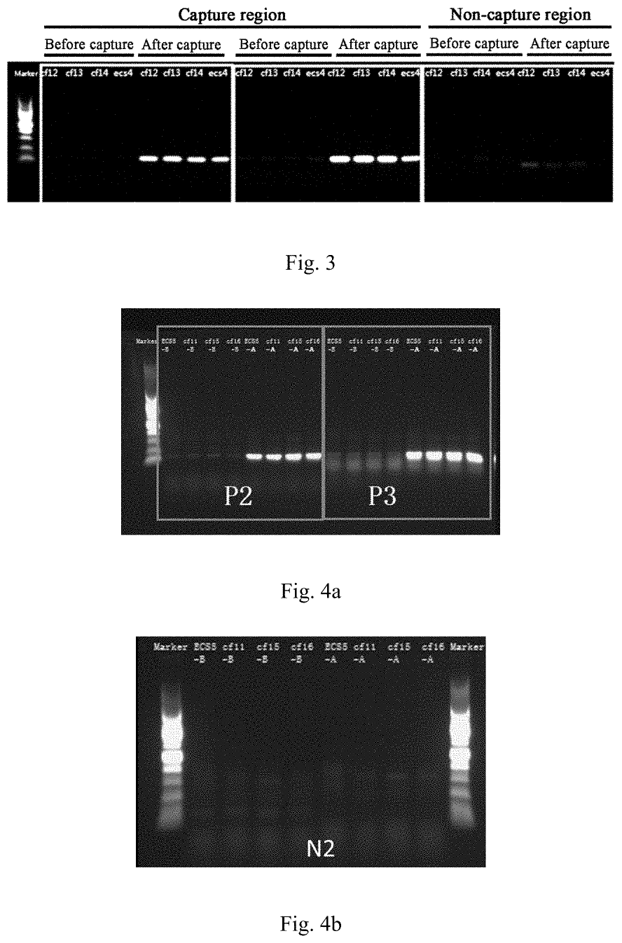

3. Quantitative analysis on comparison of enrichment degrees of the target region before and after capture. As shown in FIG. 3, FIG. 4a, FIG. 4b as well as Table 13 and Table 14, more than 20 times of DNA in the target region can be enriched after capture.

TABLE 13
DNA amounts (quantification unit)
in different lanes shown in FIG. 3

| | Batch | | | After |
|---|---|---|---|---|
| | number | | | capture/ |
| | of | Before | After | before |
| | library | capture | capture | capture |
| P2 | cf12 | 386.79 | 19429.10 | 50 |
| | cf13 | 389.54 | 20948.50 | 54 |
| | cf14 | 304.78 | 18194.50 | 60 |
| P3 | cf12 | 806.04 | 25877.00 | 32 |
| | cf13 | 1180.80 | 27208.00 | 23 |
| | cf14 | 1157.00 | 25260.60 | 22 |

TABLE 14
DNA amounts (quantification unit) in
different lanes shown in FIG. 4a

| | Batch number | Before | After | After capture/ |
|---|---|---|---|---|
| | of library | capture | capture | Before capture |
| P2 | cf11 | 948.659 | 28520.6 | 30.06 |
| | cf15 | 1540.96 | 31108.3 | 20.19 |
| | cf16 | 959.325 | 30816.3 | 32.12 |
| P3 | cf11 | 1063.97 | 41006.3 | 38.54 |
| | cf15 | 997.5 | 44393 | 44.50 |
| | cf16 | 1265.83 | 43087.1 | 34.04 |

### Example 2: Sequencing

Sequencing was performed using MGI high-throughput sequencing platform MGISEQ-2000 and a supporting reagent high-throughput sequencing set (PE100). The principle of sequencing is that sample sequence information having high quality and accuracy can be obtained by polymerizing a DNA molecule anchor and a fluorescent probe on DNA nanospheres (DNB) using a Combinatorial Probe-Anchor Synthesis (cPAS), collecting optical signals utilizing a high-resolution imaging system, and digitally processing the optical signals. The sequencing of the library amplified after capture was completed only through the following operations to output fastq files: library quantification, cyclizing, DNB preparation, high-throughput sequencing and data splitting and comparison:
1. Quality control of concentration and fragment length was performed, the concentration was determined using Invitrogen Qubit Fluorometer and a supporting reagent Qubit 1× dsDNA HS Assay Kit, and the fragment length was determined using an Aglient2100 biological analyzer and a supporting reagent Agilent DNA 1000 Reagents;
2. Cyclization: the molar mass of the library was required to ≥1 pmol. The mass (ng) corresponding to 1 pmol PCR product=main DNA fragment size (bp)×660 ng/1000 bp. The input amount was calculated according to information about concentration and fragment length in the above operation. Denaturation, single-chain cyclizing, enzyme digestion and purification were performed using MGIEsay cyclizing kit. Quantification was performed using Qubit ssDNA Assay Kit, which required that cyclizing yield was >7%, and the cyclizing yield=output of product after purification and enzyme digestion/input×100%;
3. DNA preparation: after cyclizing was completed, the concentration of initial library ssDNA was >2 fmol/μL. The input amount was 40 fmol, and the actual concentration (ng/μL) of the library was quantified using Qubit ssDNA Assay Kit and Qubit Fluorometer, and the input amount was calculated according to quantification results.

Note: input volume V (μL)=N×330 g/mol×40 mol/(1000×1000×C)

N represents the number of nucleic acids (the length of total fragments in the library), and C represents library concentration in ng/μL.

After DNB preparation was completed, quantification was performed using Qubit ssDNA Assay Kit, which required that sequencing was performed on the instrument only when DNB concentration was >8 ng/ul.

4. Data splitting and comparison: when sequencing was being performed, sequencing instrument control software automatically called the base call software for analysis, and output sequencing data fastq to a designated position for data splitting. Fastq data was aligned using bwa software (bio-bwa.sourceforge.net/) to human genome assembly build 38. Sequencing results of one batch (30) are as follows (Table 15).

TABLE 15

| | | | Concentration | | | | | | |
|---|---|---|---|---|---|---|---|---|---|
| | | | after | Cyclizing | Cyclizing | | DNB | Total | |
| Hybridization | | Number of | hybridization | concentration | output | Cyclizing | concentration | read | Q30 |
| library name | Batch | samples | (ng/ul) | (ng/μL) | (ng) | yield | (ng/ul) | (M) | (%) |
| cf62 | 30 | 30 | 25.4 | 2.14 | 42.8 | 20.27% | 15.8 | 472.18 | 90.62 |
| cf63 | 30 | 30 | 23 | 3.12 | 62.4 | 29.55% | 12.7 | 454.55 | 90.49 |
| cf64 | 30 | 30 | 28.4 | 2.26 | 45.2 | 21.40% | 15 | 448.81 | 90.14 |
| cf65 | 30 | 30 | 24.4 | 2.48 | 49.6 | 23.48% | 14.1 | 425.59 | 89.13 |

### Example 3: The Coordinative Allele-Aware Target Enrichment Improves Capture Homogeneity of Alleles in Target Region

The coordinative allele-aware target enrichment (COATE) was used to reduce the hybridization annealing temperature difference (ΔTm) between the probe and a target including reference and mutant alleles. Different from the traditional probe design, the method of designing a probe provided by the present disclosure did not require the designed probe to be complementary to the reference genome sequence or mutant sequence. These probes may or may not be complementary to the reference or mutant allele, as long as the ΔTm between the probe and the reference gene sequence (wild type) as well as mutant sequence (mutant type) in the capture region is minimized.

One example of SNP capture probe design is as follows: for SNP site rs7321990 (chr13: 20257054-20257054) on chromosome 13, there are two alleles A and G (complementary bases are T and G). Target sequences needed to be captured are as follows:

Target sequence 1:
(SEQ ID NO: 7)
TGGCGAGTTCTACCCACCTCTTGTGTTCCACCCACCGGTTCACGTCTT
CTTGTCGTCCATGAACCCTTCAGACTCCTACTGTCTTGGTTCGTCGTC
TGGGTAAGATTCGGTCCAACATTA
Target sequence 2:
(SEQ ID NO: 8)
TGGCGAGTTCTACCCACCTCTTGTGTTCCACCCACCGGTTCACGTCTT
CTTGTCGTCCACGAACCCTTCAGACTCCTACTGTCTTGGTTCGTCGTC
TGGGTAAGATTCGGTCCAACATTA

The sequence of a capture probe for capturing the target sequence can be designed as:

Capture probe 1:
(SEQ ID NO: 9)
ACCGCTCAAGATGGGTGGAGAACACAAGGTGGGTGGCCAAGTGCAGAA
GAACAGCAGGTACTTGGGAAGTCTGAGGATGACAGAACCAAGCAGCAG
ACCCATTCTAAGCCAGGTTGTAAT
Capture probe 2:
(SEQ ID NO: 10)
ACCGCTCAAGATGGGTGGAGAACACAAGGTGGGTGGCCAAGTGCAGAA
GAACAGCAGGTCCTTGGGAAGTCTGAGGATGACAGAACCAAGCAGCAG
ACCCATTCTAAGCCAGGTTGTAAT
Capture probe 3:
(SEQ ID NO: 11)
ACCGCTCAAGATGGGTGGAGAACACAAGGTGGGTGGCCAAGTGCAGAA
GAACAGCAGGTTCTTGGGAAGTCTGAGGATGACAGAACCAAGCAGCAG
ACCCATTCTAAGCCAGGTTGTAAT
Capture probe 4:
(SEQ ID NO: 12)
ACCGCTCAAGATGGGTGGAGAACACAAGGTGGGTGGCCAAGTGGAGAA
GAACAGCAGGTGCTTGGGAAGTCTGAGGATGACAGAACCAAGCAGCAG
ACCCATTCTAAGCCAGGTTGTAAT

The hybridization annealing temperatures (Tm) of the four capture probes and target sequence 1 and target sequence 2 are shown in Table 16.

TABLE 16

| | Capture | Capture | Capture | Capture |
|---|---|---|---|---|
| | probe 1 | probe 2 | probe 3 | probe 4 |
| Target | 81.678 | 81.011 | 80.952 | 81.458 |
| sequence 1 | | | | |
| Target | 81.582 | 80.694 | 81.228 | 82.017 |
| sequence 2 | | | | |
| Tm difference | 0.096 | 0.317 | 0.276 | 0.559 |
| (ΔTm) | | | | |

According to the principle that the ΔTm between the capture probe and reference gene sequence (wild type) as well as mutant sequence (mutation type) should be minimized, capture probe 1 was selected in the experiment to capture SNP site rs7321990 on chromosome 13.

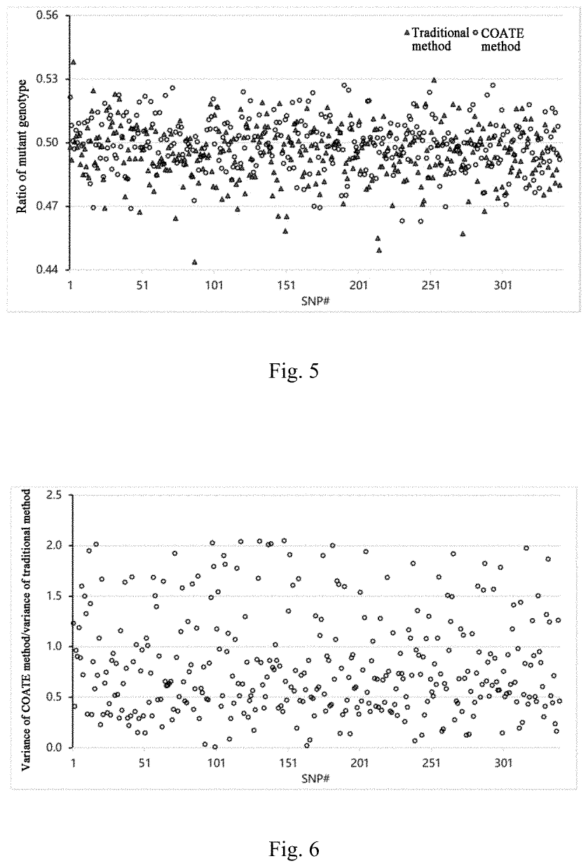

8 samples were subjected to germ-line cell free nucleic acid extraction, library construction and high-throughput sequencing, as described in example 2. The capture probe was designed using a traditional method or the coordinative allele-aware target enrichment. These 8 samples are all heterozygotes on 339 SNP sites and have the same mutation genotypes, and comparison results of mutation frequencies of the hybridized two probes for these heterozygotes are as shown in FIG. 5: for the same target region, the capture homogeneity of the alleles is improved by mutant genes obtained by using the COATE method, and the ratio of mutant genes in the heterozygote is more close to 0.5 (0.499±0.0148 vs 0.495±0.0213 95% CI);

By the COATE method, sampling bias is also reduced, the variance of the ratio of mutant genes in the heterozygote obtained by using the COATE method is 68% that of the traditional probe method. Comparison of variances of different sites is as shown in FIG. 6.

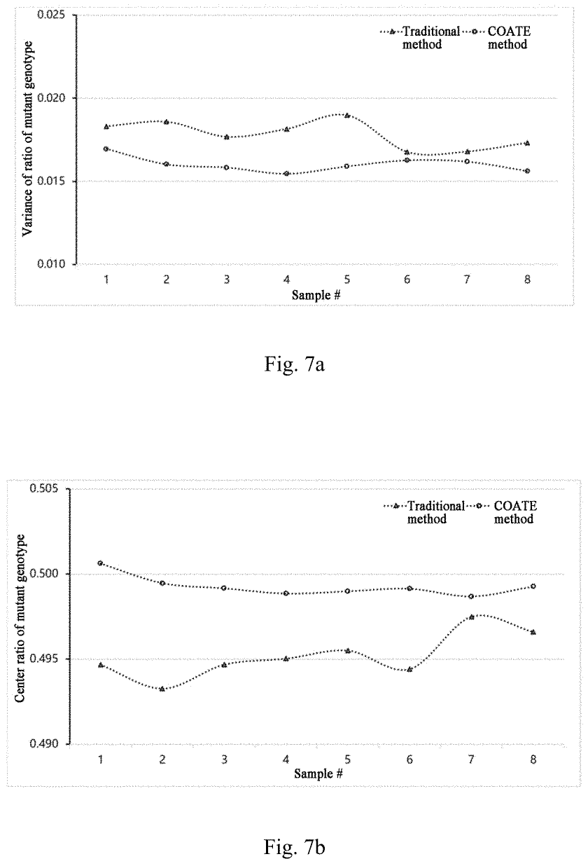

In a comparison assay of central allele fraction (CAF) of germ-line heterozygote mutation, compared with the traditional method of probe design, the probe designed based on the COATE method enriched the target region before NGS sequencing. In this group of samples, the fluctuation range of experiment error while determining CAF was significantly decreased (CAF_SDCOATE=0.0148, CAF_SDCONVENTION=0.0213, p=0.00142, 95% CI, N=8, as shown in FIG. 7a). In addition, among these 8 samples, the average CAF value of heterozygote mutation in each sample is also closer to 0.5 (CAFCOATE=0.499, CAFCONVENTION=0.495, p=0.00001, 95% CI, N=8, as shown in FIG. 7b).

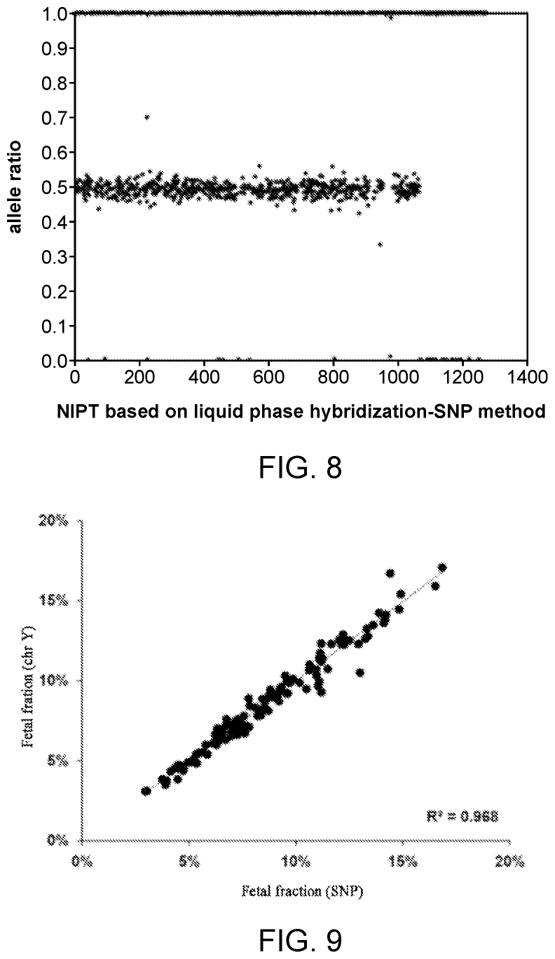

Previously, NIPS based on multiplex PCR technology has to analyze up to 20000 sites to ensure that the effective signal produced by change of maternal plasma cell-free DNA AF by fetal CNVs exceeds the change caused by experimental error of CAF. Because the fluctuation range of detection error of NGS sequencing for germ-line heterozygous CAF is reduced (as shown in FIG. 8), compared with the traditional NIPS based on multiplex PCR technology, the usage amount of probes for chromosomes 21, 18, and 13 is reduced by 60-80%. Compared with multiplex PCR, the enrichment efficiencies of the liquid hybridization technology on different transposons in the target region are more balanced.

### Example 4: Determination of the Negative Threshold of Trisomy 21 Syndrome

203 negative samples were subjected to free nucleic acid extraction, library construction and sequencing in three batches, as described in example 2. Analysis was performed via chromosome aneuploidy detection process: L(H) of diploid and different triploid fetuses was respectively calculated. The concise operations of the fetal aneuploidy detection method were as follows:
(1) detecting and calculating the fetal fraction (ff) of cell-free nucleic acids;
wherein sites in human genome where copy numbers rarely changes were selected, these sites did not include sites in chromosomes 13, 18, 21, 22 X and Y; when the mother is homozygous wild-type BB, the genotype of the fetus may be BB or BA, thus for the sites where the fetus is BA, the ratio distribution of reads A is centered on ff/2, and the fetal fraction of cell-free nucleic acids can be calculated by the median value ffBB of the ratio of reads A for all sites of this type; when the mother is homozygous mutant-type AA, the genotype of the fetus may be AA or AB, thus for the sites where the fetus is AB, the ratio distribution of reads A is centered on ff/2, and the fetal fraction of cell-free nucleic acids can be calculated by the median value ffAA of the ratio of reads B for all sites of this type; the fetal fraction (ff) of cell-free nucleic acids is calculated as:

ff=(ffAA+ffBB)/2

In a performance verification experiment for calculation of fetal DNA fraction based on the SNP method of the present disclosure, the results of the SNP method of the present disclosure and the Y chromosome calculation method in the plasmas of 128 pregnant women having male fetuses were compared. Two groups of data show high correlation (R2=0.968, as shown in FIG. 9).
(2) selecting one or more SNP sites in a chromosome to be detected;
(3) using a targeted capture probe for the one or more SNP sites to capture cell-free DNA (cfDNA) in maternal peripheral blood, and sequencing the cfDNA after amplification to obtain the reads NA of the allele A and the sequencing depth N at the site(s);
(4) calculating the probability that a fetus may have a normal chromosome copy number or abnormal different copy numbers at each SNP site; and calculating the probability values of the fetus being euploid or aneuploid, respectively, based on the percentage of mutant genotype in the cfDNA (A %) actually measured for each SNP site, the fetal fraction (ff) of cell-free nucleic acids and the mother's genotype at the site; wherein the maximum value among the sums of the probabilities at all valid SNP sites in the same chromosome is the interpreted karyotype of the fetus;
wherein the calculated fetal karyotype H includes: D (disomy), MI (maternal trisomy type I), MII (maternal trisomy type II), PI (paternal trisomy type I), PII (paternal trisomy type II), LM (maternal microdeletion) and LP (paternal microdeletion);
the karyotype probabilities of the fetus at each SNP site is obtained by taking logarithm of the linear combination of π-weighted conditional beta binomial distribution probabilities, and the calculation equation is as follows:

$$log ( p ( NAi , N , pAi , H ) ) = log ( {\sum }_{k} π k Beta - Binom ( pAi , N , α , β ) )$$

(5) calculation of chromosome copy number variation,
during sperm or egg production, if a certain chromosome under examination does not undergo meiotic homologous recombination, the calculation equation for the distribution difference between probabilities of an abnormal chromosome copy number and a normal chromosome copy number is as follows:

$$Δ L = {\sum }_{1}^{N} ( log ( LDi ) - log ( LHi ) )$$

Hϵ{MI, MII, PI, PII, LM, LP}
LD is the probability value at the site in the euploid karyotype;
LH is the probability value at the site in the aneuploid karyotype;
chromosomal aneuploidy is positive when ΔL is less than a detection threshold; the detection threshold is determined by the detection results of pregnant women's plasma samples with known prenatal diagnosis results and artificial mixtures of positive and negative reference samples.

The equations for the sum of the probabilities at the chromosomal SNP sites in the case where one chromosomal recombination may occur during the production of parental germ cells are:

$$Δ L = min ( {\sum }_{1}^{k} ( log ( LDi ) - log ( LH 1 i ) ) + {\sum }_{k + 1}^{M} ( log ( LDi ) - log ( LH 2 i ) ) ) \\ Δ L = min ( {\sum }_{1}^{k} ( log ( LDi ) - log ( LH 2 i ) ) + {\sum }_{k + 1}^{M} ( log ( LDi ) - log ( LH 1 i ) ) )$$

H1, H2ϵ{MI, MII, PI, PII}; and chromosomal aneuploidy is positive when one of the above two calculation results is less than the detection threshold.

The equations for the sum of the probabilities at the chromosomal SNP sites in the case where one or two chromosomal recombinations may occur during the production of parental germ cells are:

ΔL(H121)=min(Σ₁^{b1}(log(LDi)−log(LH1i))+Σ_{b1}^{b2}(log(LDi)−log(LH2i))+Σ_{b2}^{M}(log(LDi)−log(LH1))

ΔL(H212)=min(Σ₁^{b1}(log(LDi)−log(LH2i))+Σ_{b1}^{b2}(log(LDi)−log(LH1i))+Σ_{b2}^{M}(log(LDi)−log(LH2i))))
H1, H2ϵ{MI, MII, PI, PII},
b1 and b2 are the calculated positions where the chromosome recombinations occur; and chromosomal aneuploidy is positive when one of the above two calculation results is less than the detection threshold.

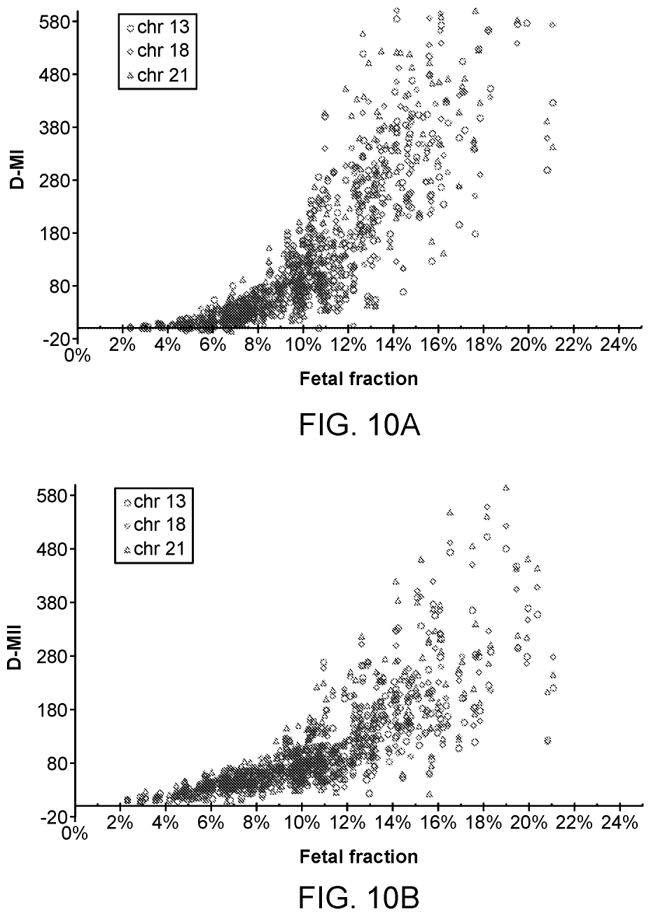

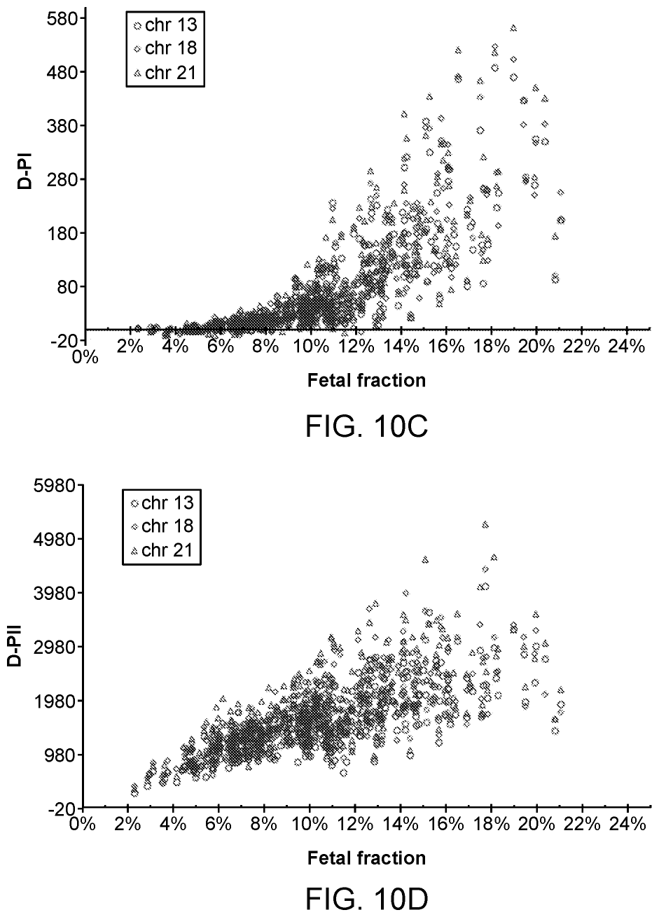

Analysis results are as shown in FIGS. 10a, 10b, 10c and 10d. Among the values of L(D)-L(H) of chromosomes 13, 18 and 21 in 203 negative samples, the difference values of 202 samples are greater than −10, and the difference value L(D)-L(PI) of one negative sample is less than −10. The conclusion is that if the negative threshold is set as −10, the false positive rate is about 0.5%.

### Example 5: Determination of the Positive Threshold of Trisomy 21 Syndrome

Positive reference sample Coriell DNA NG09394 of T21 and maternal DNA NG09387 were cut into fragments of about 180 bp using a digestion method (KAPA fragmentase, 20 min), then the fragments were mixed in the following ratios: 3%, 3.5%, 4%, 5%, 10%, 15% and 30%. Library construction was performed, and sequencing was completed, as described in example 2. Analysis was performed via chromosome aneuploidy detection process: L(H) of diploid and different triploid fetuses was respectively calculated. The operations of the detection method of fetal chromosome aneuploidy were described in example 4.

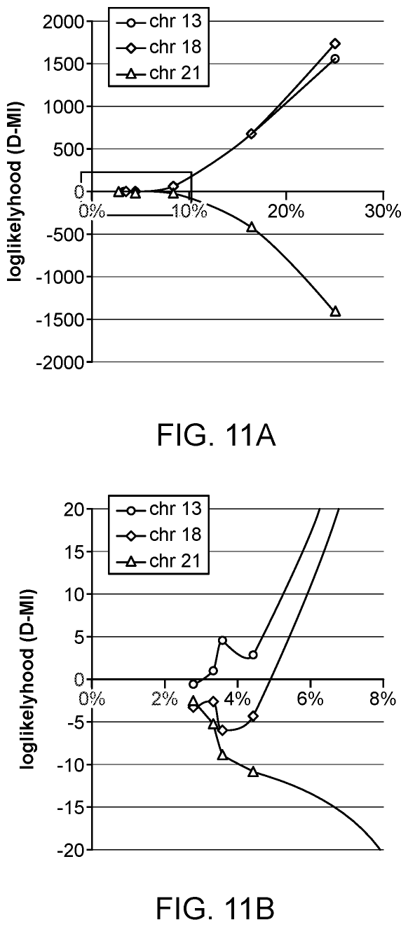

FIGS. 11a and 11b show results of analysis of T21 positive reference samples with different fetal fractions using the chromosome aneuploidy detection process. The higher the fetal fraction, the higher the L(D)-L(MI) values of chromosomes 13 and 18 of a normal diploid; while the L(D)-L(MI) value of abnormal chromosome 21 is decreased with increase of fetal fraction. If the negative threshold is set as −10 as in the example 4, abnormal chromosome 21 can be detected by this aneuploidy detection process when the fetal fraction is greater than 4%. The part in the small box in FIG. 11a is enlarged and shown in FIG. 11b.

### Example 6: Detection of Trisomy 21 Syndrome in Maternal Plasma

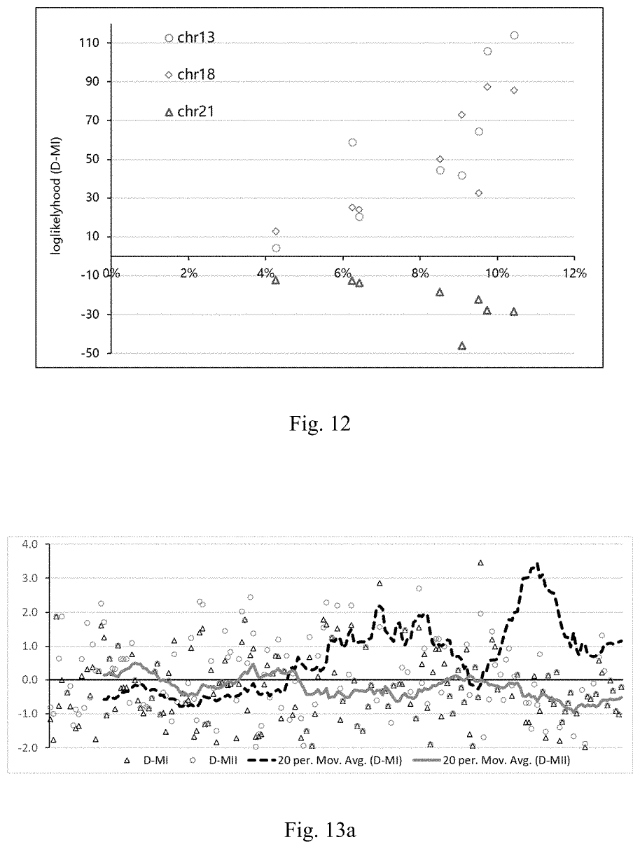

8 maternal plasma samples having trisomy 21 were subjected to extraction of cell-free nucleic acid, library construction and sequencing, as described in example 2. Analysis via chromosome aneuploidy detection process was performed as described in example 4: L(H) of diploid and triploid fetuses was respectively calculated. FIG. 12 shows results of analysis of maternal chromosome 21 positive samples with different fetal fractions using the chromosome aneuploidy detection process. The higher the fetal fraction, the higher the L(D-MI) values of chromosomes 13 and 18 of a normal diploid; and the L(D)-L(MI) value of abnormal chromosome 21 is decreased with increase of fetal fraction. If the positive threshold is set as −10, abnormal chromosome 21 can be detected by this aneuploidy detection process when the fetal fraction is greater than 4%.

### Example 7: Detection of Trisomy in which Homologous Chromosome Recombination has Occurred

Due to a long and complex life cycle of oocytes, chromosome trisomy mainly originates from the formation process of ova. At present, it is believed that there are at least three different non-disjunction modes of meiosis: homologous chromosome non-disjunction occurs during the first meiophase (MI) of the oocytes, and sister chromatid non-disjunction occurs during the second meiophase (MII) of the oocytes. The third non-disjunction mode is relatively rare and is chromosome non-disjunction during the mitosis occurring after the formation of fertilized eggs. The situation in which trisomy is formed when oocytes do not undergo chromosomal recombination is described in examples 5-6. The following example illustrates a calculation mode of trisomy syndrome formed after the oocytes undergo a chromosomal recombination. If the oocytes undergo homologous chromosome recombination during MI and homologous chromosome non-disjunction in MI occurs, it is needed to consider the mixing mode of L(MI/MII) when calculating the likelihood value of fetal trisomy; and if the oocytes undergo homologous chromosomal recombination during MI, and the sister chromatid non-disjunction in MII occurs, it is needed to consider a mixing mode of L (MII/MI) when calculating the likelihood value of fetal trisomy. Accordingly, the equations for calculating the sum of the probabilities at SNP sites on the entire chromosome are:

ΔL(MI−MII)=min(Σ₁^{k}(log(LDi)−log(LMIi)))+Σₖ₊₁^{M}(log(LDi)−log(LMIIi)))

ΔL(MII−MI)=min(Σ₁^{k}(log(LDi)−log(LMIIi))+Σₖ₊₁^{M}(log(LDi)−log(LMIi)))

The equations for the sum of the probabilities at the chromosomal SNP sites in the case where two chromosomal recombinations may occur during the production of parental germ cells are:

ΔL(H121)=min(Σ₁^{b1}(log(LDi)−log(LH1i))+Σ_{b1}^{b2}(log(LDi)−log(LH2i))+Σ_{b2}^{M}(log(LDi)−log(LH11)))

ΔL(H212)=min(Σ₁^{b1}(log(LDi)−log(LH2i))+Σ_{b1}^{b2}(log(LDi)−log(LH1i))+Σ_{b2}^{M}(log(LDi)−log(LH2i))))
H1, H2ϵ{MI, MII, PI, PII},
b1 and b2 are the calculated positions where the chromosome recombinations occur; and chromosomal aneuploidy is positive when one of the above two calculation results is less than the detection threshold shown in Table 3.

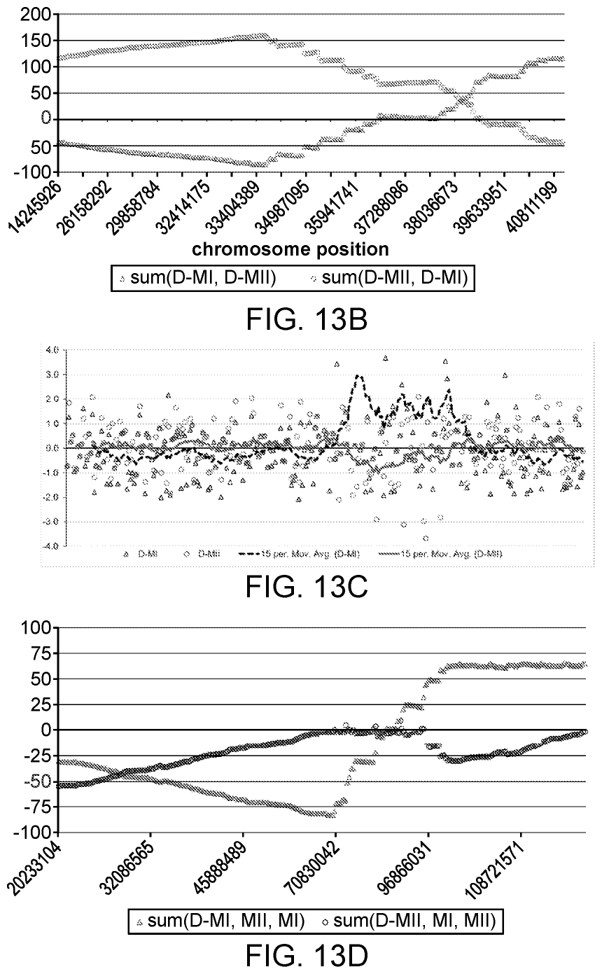

Analysis results of samples having chromosome 21 abnormality caused by one chromosome recombination are as shown in FIGS. 13a and 13b: distribution of mutant genotype ratio shows that chromosome 21 is abnormal for the possible reason that error occurs in maternal MI. The abnormal oocyte was formed with one homologous recombination on the long arm of the chromosome 21, which is consistent to the result of L(D)-L(M) moving average line (FIG. 13a). The result (FIG. 13b) of the sum of probabilities at the SNP sites of the above entire chromosome further confirms our result. Analysis results of samples having chromosome 13 abnormality caused by two chromosome recombinations are as shown in FIGS. 13c and 13d: distribution of mutant genotype ratio shows that chromosome 13 is abnormal for the possible reason that error occurs in maternal MII. The abnormal oocyte was formed with one homologous recombination on the long arm of the chromosome 13, which is consistent to the result of L(D)-L(M) moving average line (FIG. 13c). The result (FIG. 13d) of the sum of probabilities at the SNP sites of the entire chromosome further confirms our result. The conclusion is that the recombination of parental chromosomes during meiosis and a trisomy syndrome caused by non-disjunction can be analyzed and detected by this chromosome aneuploidy detection process.

### Example 8: Detection of Chromosome Microdeletion (Example of DiGeorge)

Genome DNAs obtained by nucleic acid extraction (TIANGEN genome extraction kit) of a chromosome microdeletion-positive reference cell line GM10382 (46, XY. arr [hg19] 1842.13 (227047013-227285131) x1, 22q11.21 (18876415-21465835) x1) and a maternal (normal) cell line GM10384 were cut into fragments of about 180 bp using a digestion method (KAPA fragmentase, 20 min) and then the fragments were mixed in a ratio of 10%. The cut DNAs were subjected to library construction and sequencing, as described in example 2. Analysis was performed via chromosome aneuploidy detection process: L(H) of haploid, diploid and triploid fetuses was respectively calculated. The operations of the fetal chromosome aneuploidy detection method were as described in example 4.

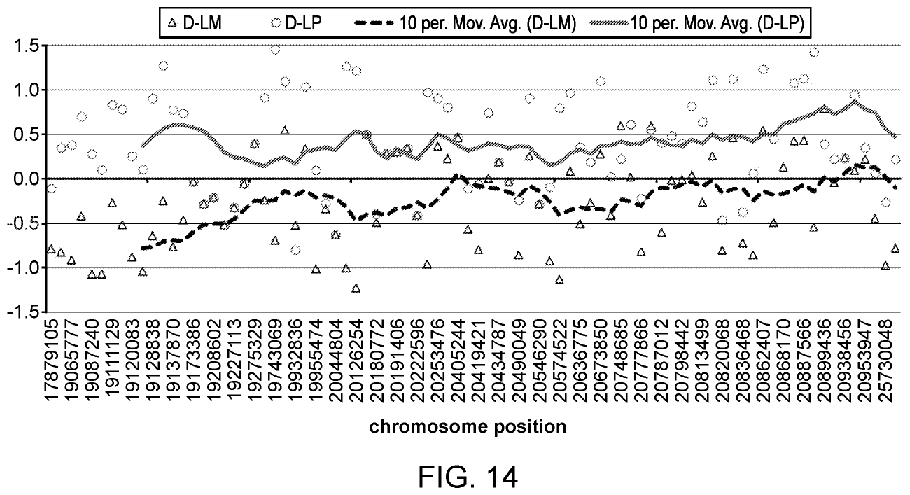

Analysis result is as shown in FIG. 14: the distribution of probability difference of mutant genotype ratios of the haploid and the diploid shows that the chromosome 22 is abnormal for the possible reason that the 22q11 region of the fetal chromosome has at least 0.5 MB microdeletion from maternal DNA, which is consistent to the result of D-LM moving average line. The statistical results in Table 17 indicate that other chromosomes of this fetus are normal, which is consistent to the result of the positive reference.

TABLE 17

| | D- | D- | D- | D- | D- | D- | D- | D- |
|---|---|---|---|---|---|---|---|---|
| Chromosomes | LM | LP | MI | MII | PI | PII | MIMII | MIIMI |
| chr13 | | | 45.9 | 53.0 | 30.3 | 893.6 | 44.1 | 49.0 |
| chr18 | | | 25.5 | 55.4 | 27.0 | 1293.7 | 30.9 | 37.3 |
| chr21 | | | 17.4 | 73.9 | 29.7 | 1226.5 | 30.4 | 30.4 |
| chr22 | −19.1 | 44.8 | | | | | | |

### Example 9: Detection of Dominant Monogenic Variation (FGFR3:.pG380R)

In two pairs of positive control, the fetal DNAs contained a pathogenic gene mutation FGFR3: c.1138G>A (p. Gly380Arg), and the maternal DNAs were normal. The genomic coordinate of this site was Chr4:1804392 (GRCh38). The fetal and maternal DNAs were cut into fragments of about 180 bp by using a digestion method (KAPA fragmentase, 20 min) and then the fragments were mixed in the following ratios: 3.5%, 5% and 10%. The cut DNAs were subjected to library construction and sequencing as well as data comparison, as described in example 2. In the detection of dominant monogenic variation, the calculation equation of the probability that the fetus has paternal or de novo mutations is as follows:

$$Δ L = log ( beta - binom ( \frac{ff}{2} , N , α , β 1 ) ) - log ( beta - binom ( e , N , α , β 2 ) )$$

N is the sequencing depth of this site, ff is the fetal fraction of cell-free nucleic acids, a is an experimental discrete parameter, β1=2×α/ff−α, and e is the system error rate of this site, and the system error rate is the ratio of mutant genotype at the site in a negative sample, namely AF value and background systematic noise,

β2=α/e−α,
ΔL is the probability of gene mutation at the site, and when ΔL is greater than the detection threshold 1, the gene mutation is positive.

The sequence of the capture probe for site FGFR3: c. 1138G>A (chr4: 1804392) is shown in Table 18.

TABLE 18

| | | |
|---|---|---|
| FGFR3: | SEQ ID | GCCTCAACGCCCATGTCTTTGCAGCCGAGGAGGAGCTGGTGG |
| c.1138 | NO: 13: | AGGCTGACGAGGGGGGCAGTGTGTATGCAGGCATCCTCAGCT |
| | | ACGGGGTGGGCTTCTTCCTGTTCATCCTGGTGGTGG |

The detection result of single gene mutation is shown in Table 19, the system error rate of 11 negative samples at the site is 0.0000448, and the probabilities ΔL of gene mutations in different positive references are all far greater than detection threshold 1.

TABLE 19

| | | Detected | | | | | | |
|---|---|---|---|---|---|---|---|---|
| | Mixing | fetal | | | | | Percentage | |
| Sample | ratio | fraction | G reads | A reads | C reads | T reads | of A | ΔL |
| 13154 | 10.0% | 9.56% | 2183 | 85 | 2 | 0 | 3.75% | 483 |
| 13155 | 5.0% | 5.61% | 2178 | 43 | 0 | 0 | 1.94% | 209 |
| 13156 | 3.5% | 4.29% | 2366 | 61 | 0 | 0 | 2.51% | 316 |
| 13157 | 10.0% | 11.54% | 2019 | 103 | 0 | 0 | 4.85% | 595 |
| 13158 | 5.0% | 6.09% | 2032 | 52 | 0 | 0 | 2.50% | 267 |
| 13159 | 3.5% | 4.40% | 1979 | 46 | 1 | 0 | 2.27% | 232 |
| 10112 | | 5.89% | 4721 | 0 | 0 | 0 | 0.00% | |
| 10114 | | 7.11% | 4059 | 1 | 0 | 1 | 0.05% | |
| 10115 | | 16.18% | 2313 | 0 | 0 | 0 | 0.00% | |
| 10141 | | 6.18% | 1908 | 0 | 0 | 0 | 0.00% | |
| 10143 | | 11.06% | 1572 | 0 | 0 | 0 | 0.00% | |
| 10192 | | 6.98% | 1628 | 0 | 0 | 0 | 0.00% | |
| 10195 | | 11.24% | 1622 | 0 | 0 | 0 | 0.00% | |
| 11586 | | 16.35% | 1019 | 0 | 0 | 0 | 0.00% | |
| 11588 | | 21.12% | 695 | 0 | 0 | 0 | 0.00% | |
| 11593 | | 21.87% | 644 | 0 | 0 | 0 | 0.00% | |
| 11594 | | 12.82% | 1242 | 0 | 0 | 0 | 0.00% | |

### Example 10: Performance Analysis of Detection of Dominant Monogenic Variation

25 pairs of fetal and maternal DNAs were cut into fragments of about 180 bp using a digestion method (KAPA fragmentase for 20 min), and then the fragments were mixed in the following ratios: 3%, 3.5%, 4%, 5%, 10%, 20%, and 30%. Library construction, sequencing and data comparison were performed, as described in example 2. The result comparison of dominant monogenic variation and fetal sequencing is shown in Table 20, and only the sites of maternal homozygous wild type are considered in the results of the list:

True positive: detected in a mixed sample, actually occurred in the fetal result.

False positive: detected in the mixed sample, not occurred in the fetal result.

True negative: not detected in the mixed sample, not occurred in the fetal result.

False negative: not detected in the mixed sample, actually occurred in the fetal result.

TABLE 20
Stimulated performance analysis of detection of single gene mutation

| Fetal fraction | 3.0% | 3.5% | 4.0% | 5.0% | 10.0% | 20.0% | 30.0% |
|---|---|---|---|---|---|---|---|
| Number of | 2 | 2 | 2 | 2 | 15 | 1 | 1 |
| samples | | | | | | | |
| Sensitivity | 100.00% | 100.00% | 100.0% | 100.00% | 99.97% | 100.00% | 100.00% |
| Specificity | 99.97% | 100.00% | 99.99% | 100.00% | 99.80% | 99.95% | 99.97% |
| Positive | 99.33% | 100.00% | 99.66% | 100.00% | 96.50% | 98.69% | 99.34% |
| prediction | | | | | | | |
| value | | | | | | | |
| Negative | 100.00% | 100.00% | 100.00% | 100.00% | 100.00% | 100.00% | 100.00% |
| prediction | | | | | | | |
| value | | | | | | | |
| True positive | 297 | 297 | 297 | 297 | 3170 | 151 | 151 |
| False positive | 2 | 0 | 1 | 0 | 115 | 2 | 1 |
| True negative | 7824 | 7824 | 7825 | 7826 | 57059 | 3937 | 3936 |
| False negative | 0 | 0 | 0 | 0 | 1 | 0 | 0 |

As described in the above Table, the fetal fraction of cell-free nucleic acids is within a range from 3.0% to 30.0%, and detection results can be obtained by using the methods of the present disclosure with extremely high sensitivity and specificity.

### Example 11: Result Analysis of Lab Performance Verification of the NIPS Technology of the Present Disclosure

Quantitative statistics was performed on the captured maternal and fetal single nucleotide polymorphisms (SNPs) in the target region through NGS. According to the above algorithm, 25 positive samples and 190 negative samples in which clinical results had been determined were detected. The positive sample detection rate is 100%, and the negative sample detection rate is 98.9% (Table 21). This result shows that the present method has high accuracy. The next operation is to expand the detection range and the quantity of detected samples to further demonstrate the performance of the present method.

TABLE 21
result of lab performance verification of the
new NIPS technology of the present disclosure

| | | Number of samples | |
|---|---|---|---|
| Type of | Number | consistent to clinical | Consistency |
| samples | of samples | detection results | rate |
| T13 | 5 | 5 | 100% |
| T18 | 3 | 3 | 100% |
| T21 | 14 | 14 | 100% |
| 22q11del | 3 | 3 | 100% |
| Negative | 190 | 188 | 98.9% |

While preferred embodiments of the present disclosure have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-64. (canceled)

65. A method of capturing nucleic acid molecules from a biological sample obtained or derived from a subject, comprising:
(a) contacting a target nucleic acid molecule obtained or derived from the biological sample with a capture probe,
wherein at least a portion of the capture probe is complementary to a target region in a reference genome to which the target nucleic acid molecule aligns, wherein the target region comprises a single nucleotide polymorphism (SNP) site, wherein the SNP site has a reference allele and an alternative allele among individuals in a reference population,
wherein the capture probe comprises a sequence selected from a set of four candidate probe sequences, wherein each of the set of four candidate probe sequences is complementary to the target region and comprises a nucleotide selected from adenine (A), thymine (T), guanine (G), and cytosine (C), respectively, at a position corresponding to the SNP site, and wherein the sequence of the capture probe is selected as a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele; and
(b) selectively hybridizing the capture probe to the target nucleic acid molecule, thereby capturing the target nucleic acid molecule.

66. The method of claim 65, wherein the target nucleic acid molecule is a cell-free nucleic acid molecule obtained from the biological sample, or an amplification product thereof.

67. The method of claim 65, wherein the target nucleic acid molecule is a cellular nucleic acid molecule obtained from the biological sample, or an amplification product thereof.

68. The method of claim 65, further comprising isolating nucleic acid molecules from the biological sample, wherein the isolated nucleic acid molecules comprise the target nucleic acid molecule.

69. The method of claim 65, further comprising amplifying nucleic acid molecules obtained or derived from the biological sample, thereby generating amplification products that comprise the target nucleic acid molecule.

70. The method of claim 65, wherein the pairing kinetics is determined at least in part by measuring a melting temperature for the first hybridizing and the second hybridizing.

71. The method of claim 65, wherein the capture probe has a length of 50 to 500 nucleotides (nt).

72. The method of claim 65, wherein the capture probe has a length of 100 to 200 nucleotides (nt).

73. The method of claim 65, wherein the capture probe has a guanine-cytosine (GC) content of 40% to 60%.

74. The method of claim 65, wherein the target region is proximal to or within one or more genes selected from the group consisting of fibroblast growth factor receptor 3 (FGFR3), FGFR2, protein tyrosine phosphatases non-receptor type 1 (PTPN11), RAF proto-oncogene serine/threonine-protein kinase (RAF1), Ras-like without CAAX 1 (RIT1), Son of sevenless homolog 1 (SOS1), collagen type I alpha 1 (COL1A1), COL1A2, COL2A1, ornithine transcarbamylase (OTC), and methyl CpG binding protein 2 (MECP2), in the reference genome.

75. The method of claim 65, wherein the capture probe is free floating in a solution.

76. The method of claim 65, wherein the capture probe is bound to a solid surface.

77. The method of claim 65, wherein the subject is a pregnant subject carrying a fetus, and wherein the method further comprises detecting a presence or an absence of a chromosomal abnormality, a chromosomal aneuploidy, a chromosomal microdeletion or microduplication, or a monogenic variant in the fetus, based at least in part on analyzing the captured target nucleic acid molecule.

78. The method of claim 77, wherein the chromosomal abnormality comprises maternal trisomy type I, maternal trisomy type II, paternal trisomy type I, paternal trisomy type II, maternal deletion, or paternal deletion.

79. The method of claim 65, further comprising sequencing the captured target nucleic acid molecule or an amplified product thereof, thereby obtaining sequence reads corresponding to the target nucleic acid molecule.

80. The method of claim 79, wherein the subject is a pregnant subject carrying a fetus, and wherein the method further comprises detecting a presence or an absence of a chromosomal abnormality, a chromosomal aneuploidy, a chromosomal microdeletion or microduplication, or a monogenic variant in the fetus, based at least in part on analyzing the sequence reads.

81. The method of claim 80, wherein the chromosomal abnormality comprises maternal trisomy type I, maternal trisomy type II, paternal trisomy type I, paternal trisomy type II, maternal deletion, or paternal deletion.

82. The method of claim 65, further comprising capturing a plurality of target nucleic acid molecules that have different nucleic acid sequences using a plurality of capture probes that have different nucleic acid sequences.

83. A method of synthesizing a capture probe, comprising:
(a) determining a target region in a reference genome to which target nucleic acid molecules align, wherein the target region comprises a single nucleotide polymorphism (SNP) site, and wherein the SNP site has a reference allele and an alternative allele among individuals in a reference population;
(b) selecting a sequence for a capture probe for the target region from a set of four candidate probe sequences, wherein each of the set of four candidate sequences is complementary to the target region and comprises a nucleotide selected from adenine (A), thymine (T), guanine (G), and cytosine (C), respectively, at a position corresponding to the SNP site, and wherein the sequence of the capture probe is selected as a sequence among the set of four candidate probe sequences that has a lowest difference in pairing kinetics between a first hybridizing of a candidate probe sequence with the target region when the SNP site has the reference allele and a second hybridizing of a candidate probe sequence with the target region when the SNP site has the alternative allele; and
(c) synthesizing the capture probe using the selected sequence.

84. A composition comprising a set of different capture probes, each different capture probe of the set of different capture probes having a sequence that is at least 80% identical to a different sequence set forth in SEQ ID NOs: 9-13.

85. A computer system, comprising:
one or more computer processors; and a non-transitory computer readable medium comprising instructions operable, when executed by the one or more computer processors, to cause the one or more computer processors to perform the method of claim 65.

86. A non-transitory computer-readable storage medium comprising instructions operable, when executed by one or more computer processors, to cause the one or more computer processors to perform the method of claim 65.


SEQUENCE LISTING (13 sequences)

SEQ ID NO: 1 - DNA, 20 bp, synthetic construct
  source 1..20: mol_type = other DNA
tgggcttcttcctgttcatc

SEQ ID NO: 2 - DNA, 19 bp, synthetic construct
  source 1..19: mol_type = other DNA
ggaagcgggagatcttgtg

SEQ ID NO: 3 - DNA, 20 bp, synthetic construct
  source 1..20: mol_type = other DNA
agctgtcacccacatcaaga

SEQ ID NO: 4 - DNA, 20 bp, synthetic construct
  source 1..20: mol_type = other DNA
ttaattgcccgtgatgttcc

SEQ ID NO: 5 - DNA, 20 bp, synthetic construct
  source 1..20: mol_type = other DNA
ggttcattaacctgggctga

SEQ ID NO: 6 - DNA, 20 bp, synthetic construct
  source 1..20: mol_type = other DNA
ctagccccaagtgagacctg

SEQ ID NO: 7 - DNA, 120 bp, synthetic construct
  source 1..120: mol_type = other DNA
tggcgagttctacccacctcttgtgttccacccaccggttcacgtcttcttgtcgtccatgaacccttcagactcctactgtcttggttcgtcgtctgggtaagattcggtccaacatta

SEQ ID NO: 8 - DNA, 120 bp, synthetic construct
  source 1..120: mol_type = other DNA
tggcgagttctacccacctcttgtgttccacccaccggttcacgtcttcttgtcgtccacgaacccttcagactcctactgtcttggttcgtcgtctgggtaagattcggtccaacatta

SEQ ID NO: 9 - DNA, 120 bp, synthetic construct
  source 1..120: mol_type = other DNA
accgctcaagatgggtggagaacacaaggtgggtggccaagtgcagaagaacagcaggtacttgggaagtctgaggatgacagaaccaagcagcagacccattctaagccaggttgtaat

SEQ ID NO: 10 - DNA, 120 bp, synthetic construct
  source 1..120: mol_type = other DNA
accgctcaagatgggtggagaacacaaggtgggtggccaagtgcagaagaacagcaggtccttgggaagtctgaggatgacagaaccaagcagcagacccattctaagccaggttgtaat

SEQ ID NO: 11 - DNA, 120 bp, synthetic construct
  source 1..120: mol_type = other DNA
accgctcaagatgggtggagaacacaaggtgggtggccaagtgcagaagaacagcaggttcttgggaagtctgaggatgacagaaccaagcagcagacccattctaagccaggttgtaat

SEQ ID NO: 12 - DNA, 120 bp, synthetic construct
  source 1..120: mol_type = other DNA
accgctcaagatgggtggagaacacaaggtgggtggccaagtgcagaagaacagcaggtgcttgggaagtctgaggatgacagaaccaagcagcagacccattctaagccaggttgtaat

SEQ ID NO: 13 - DNA, 120 bp, synthetic construct
  source 1..120: mol_type = other DNA
gcctcaacgcccatgtctttgcagccgaggaggagctggtggaggctgacgaggcgggcagtgtgtatgcaggcatcctcagctacggggtgggcttcttcctgttcatcctggtggtgg